SEQUENCE LISTING (19 sequences)

SEQ ID NO: 1 - DNA, 107 bp, Artificial Sequence
  Synthetic Construct
gaattcgtggtgagcgtctgggcatgtctgggcatgtctgggcatgtctgggcatgtcgggcattctgggcgtctgggcatgtctgggcatgtctgggcatctcgag

SEQ ID NO: 2 - DNA, 77 bp, Artificial Sequence
  Synthetic Construct
aacggtgacgtgcacgcgtgggcggagccatcacgcaggttgctatataagcagagctcgtttagtgaaccgtcaga

SEQ ID NO: 3 - DNA, 17 bp, Artificial Sequence
  Synthetic Construct
gtcgagcccgagagacc

SEQ ID NO: 4 - DNA, 4287 bp, Artificial Sequence
  Synthetic Construct
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 5 - DNA, 45 bp, Artificial Sequence
  Synthetic Construct
agagcagcataaatgttgacatgggacatttgctcatggaattgg

SEQ ID NO: 6 - DNA, 50 bp, Artificial Sequence
  Synthetic Construct
aataaagagctcagatgcatcgatcagagtgtgttggttttttgtgtgta

SEQ ID NO: 7 - DNA, 4488 bp, Artificial Sequence
  Synthetic Construct
gaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 8 - DNA, 4533 bp, Artificial Sequence
  Synthetic Construct
gaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattgg

SEQ ID NO: 9 - DNA, 145 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 10 - DNA, 145 bp, Artificial Sequence
  Synthetic Construct
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 11 - DNA, 4890 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 12 - DNA, 7767 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcagggggggggggggggggg

SEQ ID NO: 13 - protein, 725 aa, Artificial Sequence
  Synthetic Construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 14 - DNA, 95 bp, Artificial Sequence
  Synthetic Construct
gtggtgagcgtctgggcatgtctgggcatgtctgggcatgtctgggcatgtcgggcattctgggcgtctgggcatgtctgggcatgtctgggcat

SEQ ID NO: 15 - DNA, 141 bp, Artificial Sequence
  Synthetic Construct
ccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 143 bp, Artificial Sequence
  Synthetic Construct
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 17 - DNA, 4884 bp, Artificial Sequence
  Synthetic Construct
ccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 18 - DNA, 7749 bp, Artificial Sequence
  Synthetic Construct
ccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcgtggtgagcgtctgggcatgtctgggcatgtctgggcatgtctgggcatgtcgggcattctgggcgtctgggcatgtctgggcatgtctgggcatctcgagaacggtgacgtgcacgcgtgggcggagccatcacgcaggttgctatataagcagagctcgtttagtgaaccgtcagagtcgagcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggcaggcctaataaagagctcagatgcatcgatcagagtgtgttggttttttgtgtgtactgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggggggggggggggggg

SEQ ID NO: 19 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

(19) United States
(12) Patent Application Publication
(10) Pub. No.: US 2024/0115738 A1
(43) Pub. Date: Apr. 11, 2024

(54) METHODS AND COMPOSITIONS FOR TREATMENT OF CYSTIC FIBROSIS

(71) Applicant: Spirovant Sciences, Inc., Philadelphia, PA (US)

(72) Inventors: Mark SMITH, Allendale, NJ (US); Katherine EXCOFFON, Voorhees, NJ (US); Shen LIN, King of Prussia, PA (US); Madhupriya MAHANKALI, Wayne, PA (US); Eric YUEN, Los Altos, CA (US); Roland KOLBECK, Bethesda, MD (US); Matthew GLATFELTER, Wayne, PA (US)

(21) Appl. No.: 18/285,560

(22) PCT Filed: Apr. 15, 2022
(86) PCT No.: PCT/US2022/025061

§ 371 Date: Oct. 4, 2023

Related U.S. Application Data
(60) Provisional application No. 63/299,835, filed on Jan. 14, 2022.
(60) Provisional application No. 63/175,507, filed on Apr. 15, 2021.

(51) Int. Cl. A61K 48/00; A61K 31/704; A61K 45/06; A61P 11/00; C12N 15/86

(52) U.S. Cl. CPC A61K 48/0058; A61K 31/704; A61K 45/06; A61K 48/0033; A61K 48/0075; A61P 11/00; C12N 15/86; C12N 2750/14122; C12N 2750/14143; C12N 2750/14171

(57) ABSTRACT

Provided herein are methods for treatment of cystic fibrosis (CF), including for patients with class I CFTR mutations. The methods may involve administration of a recombinant adeno-associated virus (rAAV) that includes an AV.TL65 capsid protein and a polynucleotide that includes an F5 enhancer and a tg83 promoter operably linked to a CFTRΔR minigene, or a pharmaceutical composition thereof.

### CROSS-REFERENCE TO RELATED APPLICATIONS

This application claims the benefit of priority to U.S. Provisional Patent Application Ser. Nos. 63/175,507, filed on Apr. 15, 2021, and 63/299,835, filed on Jan. 14, 2022, the entire contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 13, 2022, is named 51209-028WO3_Sequence_Listing_4_12_22_ST25 and is 73,498 bytes in size.

### BACKGROUND

Gene therapy using adeno-associated virus (AAV) is an emerging treatment modality, including for treatment of single-gene defects. Cystic fibrosis (CF) is a lethal, autosomal-recessive disorder that affects at least 30,000 people in the U.S. alone, and at least 70,000 people worldwide. The average survival age for CF patients is about 40 years. CF is caused by mutations in the gene encoding the cystic fibrosis transmembrane conductance regulator (CFTR), a channel that conducts chloride and bicarbonate ions across epithelial cell membranes. Impaired CFTR function leads to inflammation of the airways and progressive bronchiectasis. Because of the single-gene etiology of CF and the various CFTR mutations in the patient population, gene therapy potentially provides a universal cure for CF.

Adeno-associated virus (AAV), a member of the human parvovirus family, is a non-pathogenic virus that depends on helper viruses for its replication. For this reason, recombinant AAV (rAAV) vectors are among the most frequently used in gene therapy pre-clinical studies and clinical trials. Indeed, CF lung disease clinical trials with rAAV2 demonstrated both a good safety profile and long persistence of the viral genome in airway tissue (as assessed by biopsy) relative to other gene transfer agents (such as recombinant adenovirus). Nevertheless, gene transfer failed to improve lung function in CF patients because transcription of the rAAV vector-derived CFTR mRNA was not detected.

Therefore, there remains a need in the art for improved compositions and methods for treatment of CF.

### SUMMARY

The disclosure provides, inter alia, methods of treating CF by administering rAAVs and/or augmenters of AAV transduction, as well as rAAVs and compositions thereof (e.g., pharmaceutical compositions) for use in the methods disclosed herein.

In one aspect, the invention features a method of treating cystic fibrosis (CF) in a subject whose genotype comprises at least one class I CFTR mutation, the method comprising administering to the subject a therapeutically effective amount of a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the invention features an rAAV for use in treating CF in a subject whose genotype comprises at least one class I CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the invention features a method of treating CF in a subject lacking CFTR protein, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the invention features an rAAV for use in treating CF in a subject lacking CFTR protein, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In some embodiments of any of the preceding aspects, the subject's genotype comprises at least one class I CFTR mutation.

In some embodiments of any of the preceding aspects, the at least one class I CFTR mutation is a nonsense mutation, a splice mutation, or a deletion.

In some embodiments of any of the preceding aspects, the at least one class I CFTR mutation comprises a Q2X mutation, a S4X mutation, a W19X mutation, a G27X mutation, a Q39X mutation, a W57X mutation, a E60X mutation, a R75X mutation, a L88X mutation, a E92X mutation, a Q98X mutation, a Y122X mutation, a E193X mutation, a W216X mutation, a L218X mutation, a Q220X mutation, a Y275X mutation, a C276X mutation, a Q290X mutation, a G330X mutation, a W401X mutation, a Q414X mutation, a S434X mutation, a S466X mutation, a S489X mutation, a Q493X mutation, a W496X mutation, a C524X mutation, a Q525X mutation, a G542X mutation, a G550X mutation, a Q552X mutation, a R553X mutation, a E585X mutation, a G673X mutation, a Q685X mutation, a R709X mutation, a K71 OX mutation, a Q715X mutation, a L732X mutation, a R764X mutation, a R785X mutation, a R792X mutation, a E822X mutation, a W882X mutation, a W846X mutation, a Y849X mutation, a R851X mutation, a Q890X mutation, a S912X mutation, a Y913X mutation, a Q1042X mutation, a W1089X mutation, a Y1092X mutation, a W1098X mutation, a R1102X mutation, a E1104X mutation, a W1145X mutation, a R1158X mutation, a R1162X mutation, a S1196X mutation, a W1204X mutation, a L1254X mutation, a S1255X mutation, a W1282X mutation, a Q1313X mutation, a Q1330X mutation, a E1371X mutation, a Q1382X mutation, a Q1411X mutation, a 2116delCTAA mutation, or a combination thereof. In some embodiments, the at least one class I mutation comprises a G542X mutation, a W1282X mutation, an R1162X mutation, an R553X mutation, a 2116delCTAA mutation, or a combination thereof.

In some embodiments of any of the preceding aspects, the subject's genotype comprises two class I CFTR mutations.

In some embodiments of any of the preceding aspects, the subject's genotype comprises a W1282X mutation and a R1162X mutation.

In one aspect, the invention features a method of treating CF in a subject whose genotype comprises at least one class III CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the invention features an rAAV for use in treating CF in a subject whose genotype comprises at least one class III CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In some embodiments of any of the preceding aspects, the at least one class III CFTR mutation comprises a G551 D mutation or a S549N mutation.

In some embodiments of any of the preceding aspects, the subject's genotype comprises two class III CFTR mutations.

In some embodiments of any of the preceding aspects, the subject's genotype comprises one class I CFTR mutation and one class III CFTR mutation.

In some embodiments of any of the preceding aspects, the method or use further comprises administering to the subject a therapeutically effective amount of an augmenter of AAV transduction.

In some embodiments of any of the preceding aspects, the augmenter is administered to the subject within about 48 h following administration of the rAAV.

In another aspect, the invention features a method of treating CF in a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 48 h following administration of the rAAV.

In another aspect, the invention features an rAAV for use in a method of treating CF in a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 48 h following administration of the rAAV.

In another aspect, the invention features a method of treating CF in a subject, the method comprising administering to the subject a therapeutically effective amount of an augmenter of AAV transduction, wherein the augmenter is administered to the subject within about 48 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the invention features an augmenter of AAV transduction for use in treating CF in a subject, wherein the augmenter is administered to the subject within about 48 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In some embodiments of any of the preceding aspects, the augmenter is administered to the subject within about 24 h following administration of the rAAV.

In some embodiments of any of the preceding aspects, the augmenter is administered to the subject within about 12 h following administration of the rAAV.

In some embodiments of any of the preceding aspects, the augmenter is a proteasome modulating agent.

In some embodiments of any of the preceding aspects, the proteasome modulating agent is an anthracycline, a proteasome inhibitor, a tripeptidyl aldehyde, or a combination thereof.

In some embodiments of any of the preceding aspects, the augmenter is an anthracycline, a proteasome inhibitor, a tripeptidyl aldehyde, or a combination thereof.

In some embodiments of any of the preceding aspects, the anthracycline is doxorubicin, idarubicin, aclarubicin, daunorubicin, epirubicin, valrubicin, mitoxantrone, or a combination thereof.

In some embodiments of any of the preceding aspects, the anthracycline is doxorubicin, idarubicin, or a combination thereof.

In some embodiments of any of the preceding aspects, the anthracycline is doxorubicin.

In some embodiments of any of the preceding aspects, the proteasome inhibitor is bortezomib, carfilzomib, and ixazomib.

In some embodiments of any of the preceding aspects, the tripeptidyl aldehyde is N-acetyl-1-leucyl-1-leucyl-1-norleucine (LLnL).

In some embodiments of any of the preceding aspects, the subject lacks CFTR protein.

In some embodiments of any of the preceding aspects, the subject's genotype comprises at least one class I CFTR mutation.

In some embodiments of any of the preceding aspects, the at least one class I CFTR mutation is a nonsense mutation, a splice mutation, or a deletion.

In some embodiments of any of the preceding aspects, the at least one class I CFTR mutation comprises a Q2X mutation, a S4X mutation, a W19X mutation, a G27X mutation, a Q39X mutation, a W57X mutation, a E60X mutation, a R75X mutation, a L88X mutation, a E92X mutation, a Q98X mutation, a Y122X mutation, a E193X mutation, a W216X mutation, a L218X mutation, a Q220X mutation, a Y275X mutation, a C276X mutation, a Q290X mutation, a G330X mutation, a W401X mutation, a Q414X mutation, a S434X mutation, a S466X mutation, a S489X mutation, a Q493X mutation, a W496X mutation, a C524X mutation, a Q525X mutation, a G542X mutation, a G550X mutation, a Q552X mutation, a R553X mutation, a E585X mutation, a G673X mutation, a Q685X mutation, a R709X mutation, a K71 OX mutation, a Q715X mutation, a L732X mutation, a R764X mutation, a R785X mutation, a R792X mutation, a E822X mutation, a W882X mutation, a W846X mutation, a Y849X mutation, a R851X mutation, a Q890X mutation, a S912X mutation, a Y913X mutation, a Q1042X mutation, a W1089X mutation, a Y1092X mutation, a W1098X mutation, a R1102X mutation, a E1104X mutation, a W1145X mutation, a R1158X mutation, a R1162X mutation, a S1196X mutation, a W1204X mutation, a L1254X mutation, a S1255X mutation, a W1282X mutation, a Q1313X mutation, a Q1330X mutation, a E1371X mutation, a Q1382X mutation, a Q1411X mutation, a 2116delCTAA mutation, or a combination thereof.

In some embodiments of any of the preceding aspects, the subject's genotype comprises two class I CFTR mutations.

In some embodiments of any of the preceding aspects, the subject's genotype comprises a W1282X mutation and a R1162X mutation.

In some embodiments of any of the preceding aspects, the subject's genotype comprises at least one class II CFTR mutation, at least one class III CFTR mutation, at least one class IV CFTR mutation, at least one class V CFTR mutation, at least one class VI CFTR mutation, or at least one class VII CFTR mutation.

In some embodiments of any of the preceding aspects, the subject's genotype comprises two class II CFTR mutations, two class III CFTR mutations, two class IV CFTR mutations, two class V CFTR mutations, two class VI CFTR mutations, or two class VII CFTR mutations.

In some embodiments of any of the preceding aspects, the rAAV comprises an AV.TL65 capsid protein.

In some embodiments of any of the preceding aspects, the AV.TL65 capsid protein comprises the amino acid sequence of SEQ ID NO:13.

In some embodiments of any of the preceding aspects, the polynucleotide comprises an F5 enhancer.

In some embodiments of any of the preceding aspects, the F5 enhancer comprises the polynucleotide sequence of SEQ ID NO:1.

In some embodiments of any of the preceding aspects, the F5 enhancer comprises the polynucleotide sequence of SEQ ID NO:14.

In some embodiments of any of the preceding aspects, the polynucleotide comprises a tg83 promoter.

In some embodiments of any of the preceding aspects, the tg83 promoter comprises the polynucleotide sequence of SEQ ID NO:2.

In some embodiments of any of the preceding aspects, the CFTRΔR minigene is a human CFTRΔR minigene.

In some embodiments of any of the preceding aspects, the human CFTRΔR minigene is encoded by a polynucleotide comprising the sequence of SEQ ID NO:4.

In some embodiments of any of the preceding aspects, the polynucleotide comprises, in a 5′-to-3′ direction, the F5 enhancer, the tg83 promoter, and the CFTRΔR minigene.

In some embodiments of any of the preceding aspects, the polynucleotide comprises the sequence of SEQ ID NO:7.

In some embodiments of any of the preceding aspects, the method or use further comprises administering one or more additional therapeutic agents to the subject.

In some embodiments of any of the preceding aspects, the one or more additional therapeutic agents includes an antibiotic, a mucus thinner, a CFTR modulator, a mucolytic, normal saline, hypertonic saline, an immunosuppressive agent, or a combination thereof.

In some embodiments of any of the preceding aspects, the administering is by inhalation, nebulization, aerosolization, intranasally, intratracheally, intrabronchially, orally, intravenously, subcutaneously, or intramuscularly.

In some embodiments of any of the preceding aspects, the administering is by inhalation, nebulization, aerosolization, intranasally, intratracheally, and/or intrabronchially.

In some embodiments of any of the preceding aspects, the administering is by inhalation.

### BRIEF DESCRIPTION OF THE DRAWINGS

FIGS. 1A and 1B show production of luciferase activity after transduction of primary human airway epithelia with the reporter AV.TL65-CBA-mCherry-SP183 for 4 hours and treatment with the augmenter doxorubicin at 2, 4, 6, and 22 hours post-AAV addition. FIG. 1A shows a schematic representing the short time-course of treatment with augmenter. FIG. 1B shows luciferase signal determined in the conditioned media of transduced HAE, non-CF and CF, at 2 and 4 DAT. For each group, n=2-4 technical replicates; *<0.05, comparing to non-treated control wells by two-tailed, unpaired T-test.

FIGS. 2A and 2B show production of luciferase activity after transduction of primary human airway epithelia with the reporter AV.TL65-CBA-mCherry-SP183 for 16 hours and treatment with the augmenter doxorubicin at 14, 16, 18, and 22 Hours post-AAV addition. FIG. 2A shows a schematic representing the intermediate time-course of treatment with augmenter. FIG. 2B shows Luciferase signal determined in the conditioned media of transduced non-CF HAE at 2 DAT. For each group, n=2-4 technical replicates; *<0.05, comparing to non-treated control wells by two-tailed, unpaired T-test.

FIGS. 3A and 3B show production of luciferase activity after transduction of primary human airway epithelia with the reporter AV.TL65-CBA-mCherry-SP183 for 16 hours and treatment with the augmenter doxorubicin at 16, 40 and 88 hours post-AAV addition. FIG. 3A shows a schematic representing the extended time-course of treatment with augmenter. FIG. 3B shows luciferase signal determined in the conditioned media of transduced non-CF HAE at 4, 6 and 8 DAT. For each group, n=2-4 technical replicates; *<0.05 comparing to non-treated control cells by two-tailed, unpaired T-test.

FIG. 4 shows that apical SP-101 demonstrated a dose-dependent functional correction of primary CF HAE. In contrast to CFTR modulators Vertex (VX)-770/661/445 that did not restore function in donors with class I mutations (W1282X/R1162X), treatment with SP-101 (multiplicity of infection (MOI) 1 K, 10K, 100K)+doxorubicin (Dox, 5 μM) significantly increased currents in a dose-dependent manner to levels similar to non-CF HAE.

FIG. 5 shows that SP-101-capsid reporter encoding mCherry transduced many epithelial cell types in CF HAE (F508del/F508del). SP-101-reporter (mCherry) showed >30% positive cells that colocalized with markers for ciliated (α-tubulin) or secretory cells (MUC5AC) or did not colocalize with any cell type markers (non-ciliated or basally-oriented cells).

FIG. 6 shows that SP-101 vector genomes were abundant in many regions of non-CF ferret lungs. SP-101 vector genomes (dots) were detected in multiple cells whereas pretreatment with DNase did not show staining, indicating the specificity of staining.

FIG. 7 shows that hCFTRΔR mRNA expression in ferret airway tissues was increased >10 fold by administration of doxorubicin. In contrast to control samples, hCFTRΔR mRNA was detected in the majority of samples from animals exposed to SP-101 alone. However, hCFTRΔR mRNA was >10 fold higher in samples from animals exposed to the same amount of SP-101 followed by doxorubicin (p<0.0001). hCFTRΔR mRNA copies were normalized to the total amount of 500 ng mRNA/sample.

FIG. 8 shows that hCFTRΔR mRNA expression was durable in non-CF ferret lungs. hCFTRΔR mRNA did not significantly decrease 12 weeks (end of study) post-administration, indicating durable expression. hCFTRΔR mRNA copies were normalized to the total amount of 500 ng mRNA/sample.

FIG. 9 shows that hCFTRΔR mRNA expression was similar in the lungs of CF and non-CF ferrets. In contrast to control animals (diluent only), hCFTRΔR mRNA was detectable to a similar extent in both CF (G551 D) and non-CF animals, indicating that the CF lung is not an additional barrier to SP-101. hCFTRΔR mRNA copies were normalized to the total amount of 500 ng mRNA/sample.

FIG. 10 shows that a low level of doxorubicin was sufficient to enhance the ability for SP-101 to demonstrate functional activity in class I CF HAE by Ussing chamber analysis.

FIG. 11 shows that vector copy number (VCN) correlated with SP-101 MOI and doxorubicin dose in class I CF HAE.

FIG. 12 shows that absolute copy number of hCFTRΔR mRNA (normalized during cDNA conversion) increased with increasing SP-101 MOI and doxorubicin dose in class I CF HAE.

FIG. 13 shows a SP-101 dose-dependent (MOI) correction of CF HAE in this class I donor at 1 μM doxorubicin.

FIG. 14 shows that all doses of SP-101 above 5e2 MOI stimulated Ussing current significantly greater than non-CF HAE in the presence of 1 μM doxorubicin.

FIG. 15 shows that a doxorubicin dose response and a SP-101 MOI dose response were observed in CF HAE from this class I CF donor.

FIG. 16 shows that increasing doxorubicin and SP-101 vector doses increased the correction of CF human airway epithelia, derived from donors with class I, II or III CFTR mutations, to levels similar to non-CF epithelia. In contrast, Vertex modulator (VX-770/661/445) treatment did not correct epithelia with two class I mutations, and only partially corrected epithelia heterozygous for class I and III mutations. The only epithelia that the Vertex modulator treatment could fully correct were epithelia with two class II mutations. Symbols represent average peak Ussing current for 3-4 epithelia from the donor indicated.

### DETAILED DESCRIPTION OF EMBODIMENTS OF THE DISCLOSURE

Gene therapy is the only mutation-agnostic approach to treat cystic fibrosis (CF). The present disclosure is based, at least in part, on the discovery that the rAAV vectors described herein (e.g., AV.TL65-SP183-hCFTRΔR) are unexpectedly effective in complementing CFTR-mediated chloride transport in polarized human CF airway epithelium, including from patients whose genotypes harbor class I mutations in the CFTR gene. Such class I mutations lead to the near absence or absence of CFTR protein, and include stop codon mutations and frameshift mutations that result in a premature termination codon. Approximately 22% of CF patients have at least one class I mutation, representing the largest class of mutations that does not have a currently approved therapy. CF caused by class I mutations is considered to be particularly difficult to treat, at least in part because it is not amenable to treatment with currently approved therapies such as correctors (e.g., lumacaftor or tezacaftor), which help defective CFTR fold correctly, or potentiators (e.g., ivacaftor), which help open the CFTR channel and increase the function of normal CFTR. Thus, based on the results disclosed herein, it is expected that the methods disclosed herein will be effective for treatment of CF caused by class I CFTR mutations, including for patients whose genotype includes one or two class I CFTR mutations. The present disclosure is also based, at least in part, on the discovery that sequential administration of an rAAV vector as disclosed herein (e.g., AV.TL65-SP183-hCFTRΔR) followed by administration of an augmenter (e.g., doxorubicin), e.g., within about 72 h, about 48 h, about 24 h, or about 12 h, results in robust gene expression in the airway of an art-accepted animal CF model. The degree of transduction was unexpectedly high in view of the pre-existing mucus accumulation in the respiratory tract of the CF ferrets.

### Definitions

The term “AAV” refers to adeno-associated virus, and may be used to refer to the naturally occurring wild-type virus itself or derivatives thereof. The term covers all subtypes, serotypes and pseudotypes, and both naturally occurring and recombinant forms, except where required otherwise. The AAV genome is built of single stranded DNA, and comprises inverted terminal repeats (ITRs) at both ends of the DNA strand, and two open reading frames: rep and cap, encoding replication and capsid proteins, respectively. A foreign polynucleotide can replace the native rep and cap genes. AAVs can be made with a variety of different serotype capsids which have varying transduction profiles, or, as used herein, “tropism” for different tissue types.

As used herein, the term “serotype” refers to an AAV which is identified by and distinguished from other AAVs based on capsid protein reactivity with defined antisera, e.g., AAV1, AAV2, AAV3, AAV4, AAV5, AAV6, AAV7, AAV8, AAV9, and AAVrh10. For example, serotype AAV2 is used to refer to an AAV which contains capsid proteins encoded from the cap gene of AAV2 and a genome containing 5′- and 3′-ITR sequences from the same AAV2 serotype. Pseudotyped AAV as refers to an AAV that contains capsid proteins from one serotype and a viral genome including 5′- and 3′-ITRs of a second serotype. Pseudotyped rAAV would be expected to have cell surface binding properties of the capsid serotype and genetic properties consistent with the ITR serotype. Pseudotyped rAAV are produced using standard techniques described in the art.

The term “about” is used herein to mean a value that is ±10% of the recited value.

As used herein, by “administering” is meant a method of giving a dosage of a composition described herein (e.g., an rAAV or a pharmaceutical composition thereof) to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), orally, nasally, rectally, topically, or buccally. In some embodiments, a composition described herein is administered in aerosolized particles intratracheally and/or intrabronchially using an atomizer sprayer (e.g., with a MADgic® laryngo-tracheal mucosal atomization device). The compositions utilized in the methods described herein can also be administered locally or systemically. The method of administration can vary depending on various factors (e.g., the components of the composition being administered and the severity of the condition being treated).

The term “AV.TL65” refers to an evolved chimeric AAV capsid protein that is highly tropic for the human airway. AV.TL65 is described in Excoffon et al. Proc. Natl. Acad. Sci. USA 106(10):3865-3870, 2009, which is incorporated by reference herein in its entirety, and is also known in the art as AAV2.5T. AV.TL65 is a chimera between AAV2 (a.a. 1-128) and AAV5 (a.a. 129-725) with one point mutation (A581T). The amino acid sequence of the AV.TL65 capsid is shown below:

(SEQ ID NO: 13)
MAADGYLPDWLEDTLSEGIRQWWKLKPGPPPPKPAERHKDDSRGLVLPGYKYLGPENGLD
KGEPVNEADAAALEHDKAYDRQLDSGDNPYLKYNHADAEFQERLKEDTSEGGNLGRAVFQ
AKKRVLEPFGLVEEGAKTAPTGKRIDDHFPKRKKARTEEDSKPSTSSDAEAGPSGSQQLQ
IPAQPASSLGADTMSAGGGGPLGDNNQGADGVGNASGDWHCDSTWMGDRVVTKSTRTWVL
PSYNNHQYREIKSGSVDGSNANAYFGYSTPWGYFDENRFHSHWSPRDWQRLINNYWGFRP
RSLRVKIFNIQVKEVTVQDSTTTIANNLTSTVQVFTDDDYQLPYVVGNGTEGCLPAFPPQ
VFTLPQYGYATLNRDNTENPTERSSFFCLEYFPSKMLRTGNNFEFTYNFEEVPFHSSFAP
SQNLFKLANPLVDQYLYRFVSTNNTGGVQFNKNLAGRYANTYKNWFPGPMGRTQGWNLGS
GVNRASVSAFATTNRMELEGASYQVPPQPNGMINNLQGSNTYALENTMIFNSQPANPGIT
ATYLEGNMLITSESETQPVNRVAYNVGGQMATNNQSSTTAPTTGTYNLQEIVPGSVWMER
DVYLQGPIWAKIPETGAHFHPSPAMGGFGLKHPPPMMLIKNTPVPGNITSFSDVPVSSFI
TQYSTGQVTVEMEWELKKENSKRWNPEIQYTNNYNDPQFVDFAPDSTGEYRTTRPIGTRY
LTRPL.

The term “class I CFTR mutation” refers to a mutation that interferes with the production of CFTR protein, including an absence or near absence of CFTR protein. Exemplary class I CFTR mutations include, e.g., nonsense mutations, splice mutations, and deletions. Approximately 22% of CF patients have at least one class I CFTR mutation. In some examples, the at least one class I CFTR mutation comprises a Q2X mutation, a S4X mutation, a W19X mutation, a G27X mutation, a Q39X mutation, a W57X mutation, a E60X mutation, a R75X mutation, a L88X mutation, a E92X mutation, a Q98X mutation, a Y122X mutation, a E193X mutation, a W216X mutation, a L218X mutation, a Q220X mutation, a Y275X mutation, a C276X mutation, a Q290X mutation, a G330X mutation, a W401X mutation, a Q414X mutation, a S434X mutation, a S466X mutation, a S489X mutation, a Q493X mutation, a W496X mutation, a C524X mutation, a Q525X mutation, a G542X mutation, a G550X mutation, a Q552X mutation, a R553X mutation, a E585X mutation, a G673X mutation, a Q685X mutation, a R709X mutation, a K710X mutation, a Q715X mutation, a L732X mutation, a R764X mutation, a R785X mutation, a R792X mutation, a E822X mutation, a W882X mutation, a W846X mutation, a Y849X mutation, a R851X mutation, a Q890X mutation, a S912X mutation, a Y913X mutation, a Q1042X mutation, a W1089X mutation, a Y1092X mutation, a W1098X mutation, a R1102X mutation, a E1104X mutation, a W1145X mutation, a R1158X mutation, a R1162X mutation, a S1196X mutation, a W1204X mutation, a L1254X mutation, a S1255X mutation, a W1282X mutation, a Q1313X mutation, a Q1330X mutation, a E1371X mutation, a Q1382X mutation, a Q1411X mutation, a 2116delCTAA mutation, a T663rfsX8 mutation, or a combination thereof. Other class I CFTR mutations are known in the art. In some examples, the subject's genotype comprises two class I CFTR mutations. The subject's genotype may include any combination of class I CFTR mutations. As one non-limiting example, in some instances, the subject's genotype comprises a W1282X mutation and a R1162X mutation.

The term “class II CFTR mutation” refers to a mutation that interferes with processing of CFTR protein. For class II mutations, CFTR protein is created, but typically misfolds, which interferes with trafficking to the cell surface. Approximately 88% of CF patients have at least one class II CFTR mutation. Exemplary class II CFTR mutations include, e.g., F508del, N1303K, and I507del.

The term “class III CFTR mutation” refers to a mutation that interferes with gating of the CFTR protein. For class III mutations, CFTR protein is created and traffics to the cell surface, but the channel gate does not open properly. Approximately 6% of CF patients have at least one class III CFTR mutation. Exemplary class III CFTR mutations include, e.g., G551D and S549N.

The term “class IV CFTR mutation” refers to a mutation that interferes with conduction of the CFTR protein. For class IV CFTR mutations, CFTR protein is created and traffics to the cell surface, but the function of the channel is defective. Approximately 6% of CF patients have at least one class IV CFTR mutation. Exemplary class IV CFTR mutations include, e.g., D1152H, R347P, and R117H.

The term “class V CFTR mutation” refers to a mutation that results in insufficient CFTR protein. For class V CFTR mutations, normal CFTR protein is created and traffics to the cell surface, but in insufficient amounts. Approximately 5% of CF patients have at least one class V CFTR mutation. Exemplary class V CFTR mutations include, e.g., 3849+10kbC→T, 2789+5G→A, and A455E.

The term “class VI CFTR mutation refers to a mutation that results in a less stable version of CFTR protein. Exemplary class VI mutations include, e.g., c. 120del23.

The term “class VII CFTR mutation refers to a mutation that results in an absence of CFTR mRNA. Exemplary class VII mutations include, e.g., dele2,3(21 kb) and 1717-1 G→A.

The numbering of the CFTR mutations described herein may be relative to a wild-type CFTR sequence (e.g., a nucleic acid or amino acid sequence). For example, the numbering of amino acid sequences of CFTR mutations may be relative to the wild-type human CFTR protein set forth in SEQ ID NO: 19 below:

(SEQ ID NO: 19)
MQRSPLEKASVVSKLFFSWTRPILRKGYRQRLELSDIYQIPSVDSADNLSEKLEREWDRE
LASKKNPKLINALRRCFFWRFMFYGIFLYLGEVTKAVQPLLLGRIIASYDPDNKEERSIA
IYLGIGLCLLFIVRTLLLHPAIFGLHHIGMQMRIAMFSLIYKKTLKLSSRVLDKISIGQL
VSLLSNNLNKFDEGLALAHFVWIAPLQVALLMGLIWELLQASAFCGLGFLIVLALFQAGL
GRMMMKYRDQRAGKISERLVITSEMIENIQSVKAYCWEEAMEKMIENLRQTELKLTRKAA
YVRYFNSSAFFFSGFFVVELSVLPYALIKGIILRKIFTTISFCIVLRMAVTRQFPWAVQT
WYDSLGAINKIQDFLQKQEYKTLEYNLTTTEVVMENVTAFWEEGFGELFEKAKQNNNNRK
TSNGDDSLFFSNFSLLGTPVLKDINFKIERGQLLAVAGSTGAGKTSLLMVIMGELEPSEG
KIKHSGRISFCSQFSWIMPGTIKENIIFGVSYDEYRYRSVIKACQLEEDISKFAEKDNIV
LGEGGITLSGGQRARISLARAVYKDADLYLLDSPFGYLDVLTEKEIFESCVCKLMANKTR
ILVTSKMEHLKKADKILILHEGSSYFYGTFSELQNLQPDFSSKLMGCDSFDQFSAERRNS
ILTETLHRFSLEGDAPVSWTETKKQSFKQTGEFGEKRKNSILNPINSIRKESIVQKTPLQ
MNGIEEDSDEPLERRLSLVPDSEQGEAILPRISVISTGPTLQARRRQSVLNLMTHSVNQG
QNIHRKITASTRKVSLAPQANLTELDIYSRRLSQETGLEISEEINEEDLKECFEDDMESI
PAVTTWNTYLRYITVHKSLIFVLIWCLVIFLAEVAASLVVLWLLGNTPLQDKGNSTHSRN
NSYAVIITSISSYYVFYIYVGVADTLLAMGFFRGLPLVHTLITVSKILHHKMLHSVLQAP
MSTLNTLKAGGILNRFSKDIAILDDLLPLTIFDFIQLLLIVIGAIAVVAVLQPYIFVATV
PVIVAFIMLRAYFLQTSQQLKQLESEGRSPIFTHLVTSLKGLWTLRAFGRQPYFETLFHK
ALNLHTANWFLYLSTLRWFQMRIEMIFVIFFIAVTFISILTTGEGEGRVGIILTLAMNIM
STLQWAVNSSIDVDSLMRSVSRVFKFIDMPTEGKPTKSTKPYKNGQLSKVMIIENSHVKK
DDIWPSGGQMTVKDLTAKYTEGGNAILENISFSISPGQRVGLLGRTGSGKSTLLSAFLRL
LNTEGEIQIDGVSWDSITLQQWRKAFGVIPQKVFIFSGTFRKNLDPYEQWSDQEIWKVAD
EVGLRSVIEQFPGKLDFVLVDGGCVLSHGHKQLMCLARSVLSKAKILLLDEPSAHLDPVT
YQIIRRTLKQAFADCTVILCEHRIEAMLECQQFLVIEENKVRQYDSIQKLLNERSLERQA
ISPSDRVKLFPHRNSSKCKSKPQIAALKEETEEEVQDTRL.

For a review describing various classes of CFTR mutations, see, e.g., De Boeck et al. Acta Paediatrica 2020; 109:893-899. Exemplary CFTR mutations belonging to the classes described above are known in the art. For example, exemplary CFTR mutations are described in the Cystic Fibrosis Mutation Database (genet.sickkids.on.ca), the CFTR2 database (Clinical and Functional Translation of CFTR; cftr2.org), and the UMD-CFTR database (see, e.g., Bareil et al. Hum. Mutat. 2020; 31(9):1011-1019).

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters (e.g., SP183, PGK, CMV, and other eukaryotic and viral promoters). In particular embodiments, the enhancer is F5. In particular embodiments, the promoter is tg83.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

A “gene” refers to a polynucleotide containing at least one open reading frame that is capable of encoding a particular protein after being transcribed and translated.

The term “gene delivery” refers to the introduction of an exogenous polynucleotide into a cell for gene transfer, and may encompass targeting, binding, uptake, transport, localization, replicon integration and expression.

The term “gene transfer” refers to the introduction of an exogenous polynucleotide into a cell which may encompass targeting, binding, uptake, transport, localization and replicon integration, but is distinct from and does not imply subsequent expression of the gene.

The term “gene expression” or “expression” refers to the process of gene transcription, translation, and post-translational modification.

A “helper virus” for AAV refers to a virus that allows AAV (e.g., wild-type AAV) to be replicated and packaged by a mammalian cell. A variety of such helper viruses for AAV are known in the art, including adenoviruses, herpes viruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

A “detectable marker gene” is a gene that allows cells carrying the gene to be specifically detected (e.g., distinguished from cells which do not carry the marker gene). A large variety of such marker genes are known in the art.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide).

“Host cells,” “cell lines,” “cell cultures,” “packaging cell line” and other such terms denote eukaryotic cells, preferably mammalian cells, most preferably human cells, useful in the present disclosure. These cells can be used as recipients for recombinant vectors, viruses or other transfer polynucleotides, and include the progeny of the original cell that was transduced. It is understood that the progeny of a single cell may not necessarily be completely identical (in morphology or in genomic complement) to the original parent cell.

An “isolated” plasmid, virus, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. The enrichment may be, e.g., a 2-fold enrichment, a 10-fold enrichment, a 100-fold enrichment, a 1000-fold enrichment, or higher.

As used herein, the term “operable linkage” or “operably linked” refers to a physical or functional juxtaposition of the components so described as to permit them to function in their intended manner. More specifically, for example, two DNA sequences operably linked means that the two DNAs are arranged (cis or trans) in such a relationship that at least one of the DNA sequences is able to exert a physiological effect upon the other sequence. For example, an enhancer and/or a promoter can be operably linked with a transgene (e.g., a therapeutic transgene, such as a CFTRΔR minigene).

“Packaging” as used herein refers to a series of subcellular events that results in the assembly and encapsidation of a viral vector, particularly an AAV vector. Thus, when a suitable vector is introduced into a packaging cell line under appropriate conditions, it can be assembled into a viral particle. Functions associated with packaging of viral vectors, particularly AAV vectors, are described herein and in the art.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the disclosure described herein that is a polynucleotide encompasses both the double-stranded form and each of two complementary single-stranded forms known or predicted to make up the double-stranded form.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component. Polypeptides such as “CFTR” and the like, when discussed in the context of gene therapy and compositions therefor, refer to the respective intact polypeptide, or any fragment or genetically engineered derivative thereof that retains the desired biochemical function of the intact protein (e.g., CFTRΔR). Similarly, references to CFTR, CFTRΔR, and other such genes for use in gene therapy (typically referred to as “transgenes” to be delivered to a recipient cell), include polynucleotides encoding the intact polypeptide or any fragment or genetically engineered derivative possessing the desired biochemical function.

By “pharmaceutical composition” is meant any composition that contains a therapeutically or biologically active agent (e.g., a polynucleotide comprising a transgene (e.g., a CFTRΔR minigene; see, e.g., Ostedgaard et al. Proc. Natl. Acad. Sci. USA 102:2952-2957, 2005, and Ostedgaard et al. Proc. Natl. Acad. Sci. USA 108(7):2921-6, 2011)), either incorporated into a viral vector (e.g., an rAAV vector) or independent of a viral vector (e.g., incorporated into a liposome, microparticle, or nanoparticle)) that is suitable for administration to a subject. Any of these formulations can be prepared by well-known and accepted methods of art. See, for example, Remington: The Science and Practice of Pharmacy (21st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

By “pharmaceutically acceptable diluent, excipient, carrier, or adjuvant” is meant a diluent, excipient, carrier, or adjuvant which is physiologically acceptable to the subject while retaining the therapeutic properties of the pharmaceutical composition with which it is administered.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

By “recombinant adeno-associated virus (AAV)” or “rAAV vector” is meant a recombinantly-produced AAV or AAV particle that comprises a polynucleotide sequence not of AAV origin (e.g., a polynucleotide comprising a transgene, which may be operably linked to one or more enhancer and/or promoters) to be delivered into a cell, either in vivo, ex vivo, or in vitro. The rAAV may use naturally occurring capsid proteins from any AAV serotype. In some embodiments, non-naturally occurring (e.g., chimeric) capsids may be used in the rAAVs described herein, e.g., AV.TL65.

By “reference” is meant any sample, standard, or level that is used for comparison purposes. A “normal reference sample” or a “wild-type reference sample” can be, for example, a sample from a subject not having the disorder (e.g., cystic fibrosis). A “positive reference” sample, standard, or value is a sample, standard, value, or number derived from a subject that is known to have a disorder (e.g., cystic fibrosis), which may be matched to a sample of a subject by at least one of the following criteria: age, weight, disease stage, and overall health.

The terms “subject” and “patient” are used interchangeably herein to refer to any mammal (e.g., a human, a primate, a cat, a dog, a ferret, a cow, a horse, a pig, a goat, a rat, or a mouse). Preferably, the subject is a human.

A “terminator” refers to a polynucleotide sequence that tends to diminish or prevent read-through transcription (i.e., it diminishes or prevents transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present disclosure are provided below.

A “therapeutic gene,” “prophylactic gene,” “target polynucleotide,” “transgene,” “gene of interest” and the like generally refer to a gene or genes to be transferred using a vector. Typically, in the context of the present disclosure, such genes are located within the rAAV vector (which vector is flanked by inverted terminal repeat (ITR) regions and thus can be replicated and encapsidated into rAAV particles). Target polynucleotides can be used in this disclosure to generate rAAV vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; (vi) polynucleotides for cancer therapy, such as E1A tumor suppressor genes or p53 tumor suppressor genes for the treatment of various cancers; and (vii) polynucleotides for gene editing (e.g., CRISPR). To effect expression of the transgene in a recipient host cell, it is preferably operably linked to a promoter, either its own or a heterologous promoter. A large number of suitable promoters are known in the art, the choice of which depends on the desired level of expression of the target polynucleotide; whether one desires constitutive expression, inducible expression, cell-specific or tissue-specific expression, etc. The rAAV vector may also contain a selectable marker. Exemplary transgenes include, without limitation, cystic fibrosis transmembrane conductance regulator (CFTR) or derivatives thereof (e.g., a CFTRΔR minigene; see, e.g., Ostedgaard et al. Proc. Natl. Acad. Sci. USA 108(7):2921-6, 2011, which is incorporated by reference herein in its entirety), α-antitrypsin, β-globin, γ-globin, tyrosine hydroxylase, glucocerebrosidase, aryl sulfatase A, factor VIII, dystrophin, erythropoietin, alpha 1-antitrypsin, surfactant protein SP-D, SP-A or SP-C, erythropoietin, or a cytokine, e.g., IFN-alpha, IFNγ, TNF, IL-1, IL-17, or IL-6, or a prophylactic protein that is an antigen such as viral, bacterial, tumor or fungal antigen, or a neutralizing antibody or a fragment thereof that targets an epitope of an antigen such as one from a human respiratory virus, e.g., influenza virus or RSV including but not limited to HBoV protein, influenza virus protein, RSV protein, or SARS protein.

By “therapeutically effective amount” is meant the amount of a composition administered to improve, inhibit, or ameliorate a condition of a subject, or a symptom of a disorder or disease, e.g., cystic fibrosis, in a clinically relevant manner. Any improvement in the subject is considered sufficient to achieve treatment. Preferably, an amount sufficient to treat is an amount that reduces, inhibits, or prevents the occurrence or one or more symptoms of cystic fibrosis or is an amount that reduces the severity of, or the length of time during which a subject suffers from, one or more symptoms of cystic fibrosis (e.g., by at least about 10%, about 20%, or about 30%, more preferably by at least about 50%, about 60%, or about 70%, and most preferably by at least about 80%, about 90%, about 95%, about 99%, or more, relative to a control subject that is not treated with a composition described herein). An effective amount of the pharmaceutical composition used to practice the methods described herein (e.g., the treatment of cystic fibrosis) varies depending upon the manner of administration and the age, body weight, and general health of the subject being treated. A physician or researcher can decide the appropriate amount and dosage regimen.

“Transduction” or “transducing” as used herein, are terms referring to a process for the introduction of an exogenous polynucleotide, e.g., a transgene in rAAV, into a host cell leading to expression of the polynucleotide, e.g., the transgene in the cell. The process generally includes 1) endocytosis of the AAV after it has bound to a cell surface receptor, 2) escape from endosomes or other intracellular compartments in the cytosol of a cell, 3) trafficking of the viral particle or viral genome to the nucleus, 4) uncoating of the virus particles, and generation of expressible double stranded AAV genome forms, including circular intermediates. The rAAV expressible double stranded form may persist as a nuclear episome or optionally may integrate into the host genome. The alteration of any or a combination of endocytosis of the AAV after it has bound to a cell surface receptor, escape from endosomes or other intracellular compartments to the cytosol of a cell, trafficking of the viral particle or viral genome to the nucleus, or uncoating of the virus particles, and generation of expressive double stranded AAV genome forms, including circular intermediates, may result in altered expression levels or persistence of expression, or altered trafficking to the nucleus, or altered types or relative numbers of host cells or a population of cells expressing the introduced polynucleotide. Altered expression or persistence of a polynucleotide introduced via rAAV can be determined by methods well known to the art including, but not limited to, protein expression, e.g., by ELISA, flow cytometry and Western blot, measurement of DNA and RNA production by hybridization assays, e.g., Northern blots, Southern blots and gel shift mobility assays, or quantitative or non-quantitative reverse transcription, polymerase chain reaction (PCR), or digital droplet PCR assays.

“Treatment” of an individual or a cell is any type of intervention in an attempt to alter the natural course of the individual or cell at the time the treatment is initiated, e.g., eliciting a prophylactic, curative or other beneficial effect in the individual. For example, treatment of an individual may be undertaken to decrease or limit the pathology caused by any pathological condition, including (but not limited to) an inherited or induced genetic deficiency (e.g., cystic fibrosis), infection by a viral, bacterial, or parasitic organism, a neoplastic or aplastic condition, or an immune system dysfunction such as autoimmunity or immunosuppression. Treatment includes (but is not limited to) administration of a composition, such as a pharmaceutical composition, and administration of compatible cells that have been treated with a composition. Treatment may be performed either prophylactically or therapeutically; that is, either prior or subsequent to the initiation of a pathologic event or contact with an etiologic agent. Treatment may reduce one or more symptoms of a pathological condition. For example, symptoms of cystic fibrosis are known in the art and include, e.g., persistent cough, wheezing, breathlessness, exercise intolerance, repeated lung infections, inflamed nasal passages or stuffy nose, foul-smelling or greasy stools, poor weight gain and growth, intestinal blockage, constipation, elevated salt concentrations in sweat, pancreatitis, and pneumonia. Detecting an improvement in, or the absence of, one or more symptoms of a disorder (e.g., cystic fibrosis), indicates successful treatment.

A “variant” refers to a polynucleotide or a polypeptide that is substantially homologous to a native or reference polynucleotide or polypeptide. For example, a variant polynucleotide may be substantially homologous to a native or reference polynucleotide, but which has a polynucleotide sequence different from that of the native or reference polynucleotide because of one or a plurality of deletions, insertions, and/or substitutions. In another example, a variant polypeptide may be substantially homologous to a native or reference polypeptide, but which has an amino acid sequence different from that of the native or reference polypeptide because of one or a plurality of deletions, insertions, and/or substitutions. Variant polypeptide-encoding polynucleotide sequences encompass sequences that comprise one or more additions, deletions, or substitutions of nucleotides when compared to a native or reference polynucleotide sequence, but that encode a variant protein or fragment thereof that retains activity. A wide variety of mutagenesis approaches are known in the art and can be applied by a person of ordinary skill in the art.

A variant polynucleotide or polypeptide sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. In some examples, a variant polynucleotide or polypeptide sequence can be at least 95%, or more, identical to a native or reference sequence. In some examples, a variant polynucleotide or polypeptide sequence can be at least 96%, or more, identical to a native or reference sequence. In some examples, a variant polynucleotide or polypeptide sequence can be at least 97%, or more, identical to a native or reference sequence. In some examples, a variant polynucleotide or polypeptide sequence can be at least 98%, or more, identical to a native or reference sequence. In some examples, a variant polynucleotide or polypeptide sequence can be at least 99%, or more, identical to a native or reference sequence. The degree of homology (percent identity) between a native and a variant sequence can be determined, for example, by comparing the two sequences using freely available computer programs commonly employed for this purpose on the world wide web (e.g., BLASTp or BLASTn with default settings).

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a transgene, may comprise a coding sequence of interest in gene therapy (such as a gene encoding a protein of therapeutic or interest), a coding sequence of interest in vaccine development (such as a polynucleotide expressing a protein, polypeptide or peptide suitable for eliciting an immune response in a mammal), and/or a selectable or detectable marker.

### Methods of Treating CF

Provided herein are methods of treating CF in a subject. In some examples, the subject's genotype may comprise at least one class I CFTR mutation. In other examples, the subject's genotype may comprise at least one class II CFTR mutation. In yet other examples, the subject's genotype may comprise at least one class III CFTR mutation. In yet other examples, the subject's genotype may comprise at least one class IV CFTR mutation. In yet other examples, the subject's genotype may comprise at least one class V CFTR mutation. In yet other examples, the subject's genotype may comprise at least one class VI CFTR mutation. In yet other examples, the subject's genotype may comprise at least one class VII CFTR mutation. The methods may include administering to the subject any of the vectors (e.g., rAAVs) disclosed herein, including AV.TL65-SP183-hCFTRΔR. The methods may also include administering to the subject an augmenter (e.g., doxorubicin). Also provided are methods that involve sequential administration of an rAAV (e.g., AV.TL65-SP183-hCFTRΔR) and an augmenter (e.g., doxorubicin), e.g., in which the augmenter is administered to the subject within about 72 h, about 48 h, about 24 h, or about 12 h following administration of the rAAV to the subject.

In one aspect, the disclosure provides a method of treating CF in a subject whose genotype comprises at least one class I CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in treating CF in a subject whose genotype comprises at least one class I CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another example, the disclosure provides a method of treating CF in a subject lacking CFTR protein, the method comprising administering to the subject a therapeutically effective amount of a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof. In some examples, the subject's genotype comprises at least one class I CFTR mutation.

In another example, the disclosure provides an rAAV for use in treating CF in a subject lacking CFTR protein, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof. In some examples, the subject's genotype comprises at least one class I CFTR mutation.

In one aspect, the disclosure provides a method of improving chloride conductance in airway (e.g., lung) epithelial cells of a subject whose genotype comprises at least one class I CFTR mutation, the method comprising administering to the subject an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in improving chloride conductance in airway (e.g., lung) epithelial cells of a subject whose genotype comprises at least one class I CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another example, the disclosure provides a method of improving chloride conductance in airway (e.g., lung) epithelial cells of a subject lacking CFTR protein, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof. In some examples, the subject's genotype comprises at least one class I CFTR mutation.

In another example, the disclosure provides an rAAV for use in improving chloride conductance in airway (e.g., lung) epithelial cells of a subject lacking CFTR protein, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof. In some examples, the subject's genotype comprises at least one class I CFTR mutation.

In some examples, the subject may have any class I CFTR mutation. In some examples, the at least one class I CFTR mutation is a nonsense mutation, a splice mutation, or a deletion. In some examples, the at least one class I CFTR mutation comprises a Q2X mutation, a S4X mutation, a W19X mutation, a G27X mutation, a Q39X mutation, a W57X mutation, a E60X mutation, a R75X mutation, a L88X mutation, a E92X mutation, a Q98X mutation, a Y122X mutation, a E193X mutation, a W216X mutation, a L218X mutation, a Q220X mutation, a Y275X mutation, a C276X mutation, a Q290X mutation, a G330X mutation, a W401X mutation, a Q414X mutation, a S434X mutation, a S466X mutation, a S489X mutation, a Q493X mutation, a W496X mutation, a C524X mutation, a Q525X mutation, a G542X mutation, a G550X mutation, a Q552X mutation, a R553X mutation, a E585X mutation, a G673X mutation, a Q685X mutation, a R709X mutation, a K710X mutation, a Q715X mutation, a L732X mutation, a R764X mutation, a R785X mutation, a R792X mutation, a E822X mutation, a W882X mutation, a W846X mutation, a Y849X mutation, a R851X mutation, a Q890X mutation, a S912X mutation, a Y913X mutation, a Q1042X mutation, a W1089X mutation, a Y1092X mutation, a W1098X mutation, a R1102X mutation, a E1104X mutation, a W1145X mutation, a R1158X mutation, a R1162X mutation, a Si 196X mutation, a W1204X mutation, a L1254X mutation, a S1255X mutation, a W1282X mutation, a Q1313X mutation, a Q1330X mutation, a E1371X mutation, a Q1382X mutation, a Q1411X mutation, a 2116delCTAA mutation, a T663rfsX8 mutation, or a combination thereof. For example, in some instances, the at least one class I mutation comprises a G542X mutation, a W1282X mutation, an R1162X mutation, an R553X mutation, a 2116delCTAA mutation, or a combination thereof. Other class I CFTR mutations are known in the art. In some examples, the subject's genotype does not comprise an R553X mutation.

In some examples, the subject's genotype comprises two class I CFTR mutations. The subject's genotype may include any combination of class I CFTR mutations, including any combination of the class I CFTR mutations listed above. As one non-limiting example, in some instances, the subject's genotype comprises a W1282X mutation and a R1162X mutation.

In another aspect, the disclosure provides a method of treating CF in a subject whose genotype comprises at least one class II CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in treating CF in a subject whose genotype comprises at least one class II CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a method of improving chloride conductance in airway (e.g., lung) epithelial cells of a subject whose genotype comprises at least one class II CFTR mutation, the method comprising administering to the subject an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in improving chloride conductance in airway (e.g., lung) epithelial cells of a subject whose genotype comprises at least one class II CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject's genotype may include any class 11 CFTR mutation. Exemplary class II CFTR mutations include, e.g., F508del, N1303K, and 1507del. In some examples, the subject's genotype does not comprise an F508del mutation. For example, in some examples, the subject's genotype includes a class II CFTR mutation that does not comprise an F580del mutation.

In some examples, the subject's genotype comprises two class II CFTR mutations. The subject's genotype may include any combination of class II CFTR mutations, including any combination of the class II CFTR mutations listed above.

In another aspect, the disclosure provides a method of treating CF in a subject whose genotype comprises at least one class III CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in treating CF in a subject whose genotype comprises at least one class III CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a method of improving chloride conductance in airway (e.g., lung) epithelial cells of a subject whose genotype comprises at least one class III CFTR mutation, the method comprising administering to the subject an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in improving chloride conductance in airway (e.g., lung) epithelial cells of a subject whose genotype comprises at least one class III CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject's genotype may include any class III CFTR mutation. Exemplary class III CFTR mutations include, e.g., G551 D and S549N.

In some examples, the subject's genotype comprises two class III CFTR mutations. The subject's genotype may include any combination of class III CFTR mutations, including any combination of the class III CFTR mutations listed above.

In another aspect, the disclosure provides a method of treating CF in a subject whose genotype comprises at least one class IV CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in treating CF in a subject whose genotype comprises at least one class IV CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a method of improving chloride conductance in airway (e.g., lung) epithelial cells of a subject whose genotype comprises at least one class IV CFTR mutation, the method comprising administering to the subject an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in improving chloride conductance in airway (e.g., lung) epithelial cells of a subject whose genotype comprises at least one class IV CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject's genotype may include any class IV CFTR mutation. Exemplary class IV CFTR mutations include, e.g., D1152H, R347P, and R117H.

In some examples, the subject's genotype comprises two class IV CFTR mutations. The subject's genotype may include any combination of class IV CFTR mutations, including any combination of the class IV CFTR mutations listed above.

In another aspect, the disclosure provides a method of treating CF in a subject whose genotype comprises at least one class V CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in treating CF in a subject whose genotype comprises at least one class V CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a method of improving chloride conductance in airway (e.g., lung) epithelial cells of a subject whose genotype comprises at least one class V CFTR mutation, the method comprising administering to the subject an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in improving chloride conductance in airway (e.g., lung) epithelial cells of a subject whose genotype comprises at least one class V CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject's genotype may include any class V CFTR mutation. Exemplary class V CFTR mutations include, e.g., 3849+10kbC→T, 2789+5G→A, and A455E.

In some examples, the subject's genotype comprises two class V CFTR mutations. The subject's genotype may include any combination of class V CFTR mutations, including any combination of the class V CFTR mutations listed above.

In another aspect, the disclosure provides a method of treating CF in a subject whose genotype comprises at least one class VI CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in treating CF in a subject whose genotype comprises at least one class VI CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a method of improving chloride conductance in airway (e.g., lung) epithelial cells of a subject whose genotype comprises at least one class VI CFTR mutation, the method comprising administering to the subject an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in improving chloride conductance in airway (e.g., lung) epithelial cells of a subject whose genotype comprises at least one class VI CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject's genotype may include any class VI CFTR mutation. Exemplary class VI mutations include, e.g., c. 120del23.

In some examples, the subject's genotype comprises two class VI CFTR mutations. The subject's genotype may include any combination of class VI CFTR mutations, including any combination of the class VI CFTR mutations listed above.

In another aspect, the disclosure provides a method of treating CF in a subject whose genotype comprises at least one class VII CFTR mutation, the method comprising administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in treating CF in a subject whose genotype comprises at least one class VII CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a method of improving chloride conductance in airway (e.g., lung) epithelial cells of a subject whose genotype comprises at least one class VII CFTR mutation, the method comprising administering to the subject an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an rAAV for use in improving chloride conductance in airway (e.g., lung) epithelial cells of a subject whose genotype comprises at least one class VII CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject's genotype may include any class VII CFTR mutation. Exemplary class VII mutations include, e.g., dele2,3(21kb) and 1717-1 G→A.

In some examples, the subject's genotype comprises two class VII CFTR mutations. The subject's genotype may include any combination of class VII CFTR mutations, including any combination of the class VII CFTR mutations listed above.

In some examples, the subject may have any combination of class I, class II, class III, class IV, class V, class VI, or class VII CFTR mutations.

In some examples, the subject's genotype comprises one class I CFTR mutation (including any class I CFTR mutation disclosed herein or known in the art) and one class III CFTR mutation (including any class III CFTR mutation disclosed herein or known in the art).

In some examples, the method further comprises administering to the subject a therapeutically effective amount of an augmenter of AAV transduction, e.g., any augmenter described herein. In some examples, the augmenter is doxorubicin, e.g., doxorubicin-HCl.

In some examples, the augmenter is administered to the subject within about 72 h (e.g., within about 48 h, within about 24 h, or within about 12 h) following administration of the rAAV.

In another aspect, the disclosure provides a method of treating CF in a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 72 h following administration of the rAAV.

In another aspect, the disclosure provides an rAAV for use in a method of treating CF in a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 72 h following administration of the rAAV.

In another aspect, the disclosure provides a method of improving chloride conductance in airway (e.g., lung) epithelial cells of a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 72 h following administration of the rAAV.

In another aspect, the disclosure provides an rAAV for use in a method of improving chloride conductance in airway (e.g., lung) epithelial cells of a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 72 h following administration of the rAAV.

In another aspect, the disclosure provides a method of treating CF in a subject, the method comprising administering to the subject a therapeutically effective amount of an augmenter of AAV transduction, wherein the augmenter is administered to the subject within about 72 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an augmenter of AAV transduction for use in treating CF in a subject, wherein the augmenter is administered to the subject within about 72 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides a method of improving chloride conductance in airway (e.g., lung) epithelial cells of a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 72 h following administration of the rAAV.

In another aspect, the disclosure provides an rAAV for use in a method of improving chloride conductance in airway (e.g., lung) epithelial cells of a subject, the method comprising: (a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and (b) administering to the subject a therapeutically effective amount of an augmenter of AAV transduction within about 72 h following administration of the rAAV.

In another aspect, the disclosure provides a method of improving chloride conductance in airway (e.g., lung) epithelial cells of a subject, the method comprising administering to the subject a therapeutically effective amount of an augmenter of AAV transduction, wherein the augmenter is administered to the subject within about 72 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In another aspect, the disclosure provides an augmenter of AAV transduction for use in improving chloride conductance in airway (e.g., lung) epithelial cells of a subject, wherein the augmenter is administered to the subject within about 72 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

For example, in some examples of any of the methods described herein, the augmenter may be administered to the subject within about 88 h, 72 h, about 71 h, about 70 h, about 69 h, about 68 h, about 67 h, about 66 h, about 65 h, about 64 h, about 63 h, about 62 h, about 61 h, about 60 h, about 59 h, about 58 h, about 57 h, about 56 h, about 55 h, about 54 h, about 53 h, about 52 h, about 51 h, about 50 h, about 49 h, about 48 h, about 47 h, about 46 h, about 45 h, about 44 h, about 43 h, about 42 h, about 41 h, about 40 h, about 39 h, about 38 h, about 37 h, about 36 h, about 35 h, about 34 h, about 33 h, about 32 h, about 31 h, about 30 h, about 29 h, about 28 h, about 27 h, about 26 h, about 25 h, about 24 h, about 23 h, about 22 h, about 21 h, about 20 h, about 19 h, about 18 h, about 17 h, about 16 h, about 15 h, about 14 h, about 13 h, about 12 h, about 11 h, about 10 h, about 9 h, about 8 h, about 7 h, about 6 h, about 5 h, about 4 h, about 3 h, about 2 h, or about 1 h following administration of the rAAV.

In some examples, the augmenter is administered to the subject within about 48 h following administration of the rAAV.

In some examples, the augmenter is administered to the subject within about 40 h following administration of the rAAV.

In some examples, the augmenter is administered to the subject within about 24 h following administration of the rAAV.

In some examples, the augmenter is administered to the subject within about 12 h following administration of the rAAV.

In some examples, the augmenter is administered to the subject within about 1 h to about 72 h, about 1 h to about 70 h, about 1 h to about 68 h, about 1 h to about 66 h, about 1 h to about 64 h, about 1 h to about 62 h, about 1 h to about 60 h, about 1 h to about 58 h, about 1 h to about 56 h, about 1 h to about 54 h, about 1 h to about 52 h, about 1 h to about 50 h, about 1 h to about 48 h, about 1 h to about 46 h, about 1 h to about 44 h, about 1 h to about 42 h, about 1 h to about 40 h, about 1 h to about 38 h, about 1 h to about 36 h, about 1 h to about 34 h, about 1 h to about 32 h, about 1 h to about 30 h, about 1 h to about 28 h, about 1 h to about 26 h, about 1 h to about 24 h, about 1 h to about 22 h, about 1 h to about 20 h, about 1 h to about 18 h, about 1 h to about 16 h, about 1 h to about 14 h, about 1 h to about 12 h, about 1 h to about 10 h, about 1 h to about 8 h, about 1 h to about 6 h, about 1 h to about 4 h, about 1 h to about 3 h, about 1 h to about 2 h, about 2 h to about 72 h, about 2 h to about 70 h, about 2 h to about 68 h, about 2 h to about 66 h, about 2 h to about 64 h, about 2 h to about 62 h, about 2 h to about 60 h, about 2 h to about 58 h, about 2 h to about 56 h, about 2 h to about 54 h, about 2 h to about 52 h, about 2 h to about 50 h, about 2 h to about 48 h, about 2 h to about 46 h, about 2 h to about 44 h, about 2 h to about 42 h, about 2 h to about 40 h, about 2 h to about 38 h, about 2 h to about 36 h, about 2 h to about 34 h, about 2 h to about 32 h, about 2 h to about 30 h, about 2 h to about 28 h, about 2 h to about 26 h, about 2 h to about 24 h, about 2 h to about 22 h, about 2 h to about 20 h, about 2 h to about 18 h, about 2 h to about 16 h, about 2 h to about 14 h, about 2 h to about 12 h, about 2 h to about 10 h, about 2 h to about 8 h, about 2 h to about 6 h, about 2 h to about 4 h, about 2 h to about 3 h, about 4 h to about 72 h, about 4 h to about 70 h, about 4 h to about 68 h, about 4 h to about 66 h, about 4 h to about 64 h, about 4 h to about 62 h, about 4 h to about 60 h, about 4 h to about 58 h, about 4 h to about 56 h, about 4 h to about 54 h, about 4 h to about 52 h, about 4 h to about 50 h, about 4 h to about 48 h, about 4 h to about 46 h, about 4 h to about 44 h, about 4 h to about 42 h, about 4 h to about 40 h, about 4 h to about 38 h, about 4 h to about 36 h, about 4 h to about 34 h, about 4 h to about 32 h, about 4 h to about 30 h, about 4 h to about 28 h, about 4 h to about 26 h, about 4 h to about 24 h, about 4 h to about 22 h, about 4 h to about 20 h, about 4 h to about 18 h, about 4 h to about 16 h, about 4 h to about 14 h, about 4 h to about 12 h, about 4 h to about 10 h, about 4 h to about 8 h, about 4 h to about 6 h, about 6 h to about 72 h, about 6 h to about 70 h, about 6 h to about 68 h, about 6 h to about 66 h, about 6 h to about 64 h, about 6 h to about 62 h, about 6 h to about 60 h, about 6 h to about 58 h, about 6 h to about 56 h, about 6 h to about 54 h, about 6 h to about 52 h, about 6 h to about 50 h, about 6 h to about 48 h, about 6 h to about 46 h, about 6 h to about 44 h, about 6 h to about 42 h, about 6 h to about 40 h, about 6 h to about 38 h, about 6 h to about 36 h, about 6 h to about 34 h, about 6 h to about 32 h, about 6 h to about 30 h, about 6 h to about 28 h, about 6 h to about 26 h, about 6 h to about 24 h, about 6 h to about 22 h, about 6 h to about 20 h, about 6 h to about 18 h, about 6 h to about 16 h, about 6 h to about 14 h, about 6 h to about 12 h, about 6 h to about 10 h, about 6 h to about 8 h, about 8 h to about 72 h, about 8 h to about 70 h, about 8 h to about 68 h, about 8 h to about 66 h, about 8 h to about 64 h, about 8 h to about 62 h, about 8 h to about 60 h, about 8 h to about 58 h, about 8 h to about 56 h, about 8 h to about 54 h, about 8 h to about 52 h, about 8 h to about 50 h, about 8 h to about 48 h, about 8 h to about 46 h, about 8 h to about 44 h, about 8 h to about 42 h, about 8 h to about 40 h, about 8 h to about 38 h, about 8 h to about 36 h, about 8 h to about 34 h, about 8 h to about 32 h, about 8 h to about 30 h, about 8 h to about 28 h, about 8 h to about 26 h, about 8 h to about 24 h, about 8 h to about 22 h, about 8 h to about 20 h, about 8 h to about 18 h, about 8 h to about 16 h, about 8 h to about 14 h, about 8 h to about 12 h, about 8 h to about 10 h, about 10 h to about 72 h, about 10 h to about 70 h, about 10 h to about 68 h, about 10 h to about 66 h, about 10 h to about 64 h, about 10 h to about 62 h, about 10 h to about 60 h, about 10 h to about 58 h, about 10 h to about 56 h, about 10 h to about 54 h, about 10 h to about 52 h, about 10 h to about 50 h, about 10 h to about 48 h, about 10 h to about 46 h, about 10 h to about 44 h, about 10 h to about 42 h, about 10 h to about 40 h, about 10 h to about 38 h, about 10 h to about 36 h, about 10 h to about 34 h, about 10 h to about 32 h, about 10 h to about 30 h, about 10 h to about 28 h, about 10 h to about 26 h, about 10 h to about 24 h, about 10 h to about 22 h, about 10 h to about 20 h, about 10 h to about 18 h, about 10 h to about 16 h, about 10 h to about 14 h, about 10 h to about 12 h, about 12 h to about 72 h, about 12 h to about 70 h, about 12 h to about 68 h, about 12 h to about 66 h, about 12 h to about 64 h, about 12 h to about 62 h, about 12 h to about 60 h, about 12 h to about 58 h, about 12 h to about 56 h, about 12 h to about 54 h, about 12 h to about 52 h, about 12 h to about 50 h, about 12 h to about 48 h, about 12 h to about 46 h, about 12 h to about 44 h, about 12 h to about 42 h, about 12 h to about 40 h, about 12 h to about 38 h, about 12 h to about 36 h, about 12 h to about 34 h, about 12 h to about 32 h, about 12 h to about 30 h, about 12 h to about 28 h, about 12 h to about 26 h, about 12 h to about 24 h, about 12 h to about 22 h, about 12 h to about 20 h, about 12 h to about 18 h, about 12 h to about 16 h, about 12 h to about 14 h, about 14 h to about 72 h, about 14 h to about 70 h, about 14 h to about 68 h, about 14 h to about 66 h, about 14 h to about 64 h, about 14 h to about 62 h, about 14 h to about 60 h, about 14 h to about 58 h, about 14 h to about 56 h, about 14 h to about 54 h, about 14 h to about 52 h, about 14 h to about 50 h, about 14 h to about 48 h, about 14 h to about 46 h, about 14 h to about 44 h, about 14 h to about 42 h, about 14 h to about 40 h, about 14 h to about 38 h, about 14 h to about 36 h, about 14 h to about 34 h, about 14 h to about 32 h, about 14 h to about 30 h, about 14 h to about 28 h, about 14 h to about 26 h, about 14 h to about 24 h, about 14 h to about 22 h, about 14 h to about 20 h, about 14 h to about 18 h, about 14 h to about 16 h, about 16 h to about 72 h, about 16 h to about 70 h, about 16 h to about 68 h, about 16 h to about 66 h, about 16 h to about 64 h, about 16 h to about 62 h, about 16 h to about 60 h, about 16 h to about 58 h, about 16 h to about 56 h, about 16 h to about 54 h, about 16 h to about 52 h, about 16 h to about 50 h, about 16 h to about 48 h, about 16 h to about 46 h, about 16 h to about 44 h, about 16 h to about 42 h, about 16 h to about 40 h, about 16 h to about 38 h, about 16 h to about 36 h, about 16 h to about 34 h, about 16 h to about 32 h, about 16 h to about 30 h, about 16 h to about 28 h, about 16 h to about 26 h, about 16 h to about 24 h, about 16 h to about 22 h, about 16 h to about 20 h, about 16 h to about 18 h, about 18 h to about 72 h, about 18 h to about 70 h, about 18 h to about 68 h, about 18 h to about 66 h, about 18 h to about 64 h, about 18 h to about 62 h, about 18 h to about 60 h, about 18 h to about 58 h, about 18 h to about 56 h, about 18 h to about 54 h, about 18 h to about 52 h, about 18 h to about 50 h, about 18 h to about 48 h, about 18 h to about 46 h, about 18 h to about 44 h, about 18 h to about 42 h, about 18 h to about 40 h, about 18 h to about 38 h, about 18 h to about 36 h, about 18 h to about 34 h, about 18 h to about 32 h, about 18 h to about 30 h, about 18 h to about 28 h, about 18 h to about 26 h, about 18 h to about 24 h, about 18 h to about 22 h, about 18 h to about 20 h, about 20 h to about 72 h, about 20 h to about 70 h, about 20 h to about 68 h, about 20 h to about 66 h, about 20 h to about 64 h, about 20 h to about 62 h, about 20 h to about 60 h, about 20 h to about 58 h, about 20 h to about 56 h, about 20 h to about 54 h, about 20 h to about 52 h, about 20 h to about 50 h, about 20 h to about 48 h, about 20 h to about 46 h, about 20 h to about 44 h, about 20 h to about 42 h, about 20 h to about 40 h, about 20 h to about 38 h, about 20 h to about 36 h, about 20 h to about 34 h, about 20 h to about 32 h, about 20 h to about 30 h, about 20 h to about 28 h, about 20 h to about 26 h, about 20 h to about 24 h, about 20 h to about 22 h, about 22 h to about 72 h, about 22 h to about 70 h, about 22 h to about 68 h, about 22 h to about 66 h, about 22 h to about 64 h, about 22 h to about 62 h, about 22 h to about 60 h, about 22 h to about 58 h, about 22 h to about 56 h, about 22 h to about 54 h, about 22 h to about 52 h, about 22 h to about 50 h, about 22 h to about 48 h, about 22 h to about 46 h, about 22 h to about 44 h, about 22 h to about 42 h, about 22 h to about 40 h, about 22 h to about 38 h, about 22 h to about 36 h, about 22 h to about 34 h, about 22 h to about 32 h, about 22 h to about 30 h, about 22 h to about 28 h, about 22 h to about 26 h, about 22 h to about 24 h, about 24 h to about 72 h, about 24 h to about 70 h, about 24 h to about 68 h, about 24 h to about 66 h, about 24 h to about 64 h, about 24 h to about 62 h, about 24 h to about 60 h, about 24 h to about 58 h, about 24 h to about 56 h, about 24 h to about 54 h, about 24 h to about 52 h, about 24 h to about 50 h, about 24 h to about 48 h, about 24 h to about 46 h, about 24 h to about 44 h, about 24 h to about 42 h, about 24 h to about 40 h, about 24 h to about 38 h, about 24 h to about 36 h, about 24 h to about 34 h, about 24 h to about 32 h, about 24 h to about 30 h, about 24 h to about 28 h, about 24 h to about 26 h, about 28 h to about 72 h, about 28 h to about 70 h, about 28 h to about 68 h, about 28 h to about 66 h, about 28 h to about 64 h, about 28 h to about 62 h, about 28 h to about 60 h, about 28 h to about 58 h, about 28 h to about 56 h, about 28 h to about 54 h, about 28 h to about 52 h, about 28 h to about 50 h, about 28 h to about 48 h, about 28 h to about 46 h, about 28 h to about 44 h, about 28 h to about 42 h, about 28 h to about 40 h, about 28 h to about 38 h, about 28 h to about 36 h, about 28 h to about 34 h, about 28 h to about 32 h, about 28 h to about 30 h, about 32 h to about 72 h, about 32 h to about 70 h, about 32 h to about 68 h, about 32 h to about 66 h, about 32 h to about 64 h, about 32 h to about 62 h, about 32 h to about 60 h, about 32 h to about 58 h, about 32 h to about 56 h, about 32 h to about 54 h, about 32 h to about 52 h, about 32 h to about 50 h, about 32 h to about 48 h, about 32 h to about 46 h, about 32 h to about 44 h, about 32 h to about 42 h, about 32 h to about 40 h, about 32 h to about 38 h, about 32 h to about 36 h, about 32 h to about 34 h, about 36 h to about 72 h, about 36 h to about 70 h, about 36 h to about 68 h, about 36 h to about 66 h, about 36 h to about 64 h, about 36 h to about 62 h, about 36 h to about 60 h, about 36 h to about 58 h, about 36 h to about 56 h, about 36 h to about 54 h, about 36 h to about 52 h, about 36 h to about 50 h, about 36 h to about 48 h, about 36 h to about 46 h, about 36 h to about 44 h, about 36 h to about 42 h, about 36 h to about 40 h, about 36 h to about 38 h, about 40 h to about 72 h, about 40 h to about 70 h, about 40 h to about 68 h, about 40 h to about 66 h, about 40 h to about 64 h, about 40 h to about 62 h, about 40 h to about 60 h, about 40 h to about 58 h, about 40 h to about 56 h, about 40 h to about 54 h, about 40 h to about 52 h, about 40 h to about 50 h, about 40 h to about 48 h, about 40 h to about 46 h, about 40 h to about 44 h, about 40 h to about 42 h, about 44 h to about 72 h, about 44 h to about 70 h, about 44 h to about 68 h, about 44 h to about 66 h, about 44 h to about 64 h, about 44 h to about 62 h, about 44 h to about 60 h, about 44 h to about 58 h, about 44 h to about 56 h, about 44 h to about 54 h, about 44 h to about 52 h, about 44 h to about 50 h, about 44 h to about 48 h, about 44 h to about 46 h, about 48 h to about 72 h, about 48 h to about 70 h, about 48 h to about 68 h, about 48 h to about 66 h, about 48 h to about 64 h, about 48 h to about 62 h, about 48 h to about 60 h, about 48 h to about 58 h, about 48 h to about 56 h, about 48 h to about 54 h, about 48 h to about 52 h, about 48 h to about 50 h, about 52 h to about 72 h, about 52 h to about 70 h, about 52 h to about 68 h, about 52 h to about 66 h, about 52 h to about 64 h, about 52 h to about 62 h, about 52 h to about 60 h, about 52 h to about 58 h, about 52 h to about 56 h, about 52 h to about 54 h, about 56 h to about 72 h, about 56 h to about 70 h, about 56 h to about 68 h, about 56 h to about 66 h, about 56 h to about 64 h, about 56 h to about 62 h, about 56 h to about 60 h, about 56 h to about 58 h, about 60 h to about 72 h, about 60 h to about 70 h, about 60 h to about 68 h, about 60 h to about 66 h, about 60 h to about 64 h, about 60 h to about 62 h, about 64 h to about 72 h, about 64 h to about 70 h, about 64 h to about 68 h, about 64 h to about 66 h, about 68 h to about 72 h, about 68 h to about 70 h, or about 70 h to about 72 h, following administration of the rAAV.

Any suitable augmenter may be used. In some examples, the augmenter is a proteasome modulating agent. In some examples, the augmenter is an anthracycline, a proteasome inhibitor, a tripeptidyl aldehyde, or a combination thereof. In some examples, the anthracycline is doxorubicin, idarubicin, aclarubicin, daunorubicin, epirubicin, valrubicin, mitoxantrone, or a combination thereof. In some examples, the anthracycline is doxorubicin, idarubicin, or a combination thereof. In some examples, the anthracycline is doxorubicin. In some examples, the proteasome inhibitor is bortezomib, carfilzomib, and ixazomib. In some examples, the tripeptidyl aldehyde is N-acetyl-1-leucyl-1-leucyl-1-norleucine (LLnL). In particular examples, the augmenter is doxorubicin, e.g., doxorubicin-HCl.

In some examples, the subject's genotype may comprise at least one class I CFTR mutation. In some examples, the at least one class I CFTR mutation is a nonsense mutation, a splice mutation, or a deletion.

In some examples, the at least one class I CFTR mutation comprises a G542X mutation, a W1282X mutation, an R1162X mutation, an R553X mutation, or a combination thereof. In some examples, the subject's genotype does not comprise an R553X mutation.

In some examples, the subject's genotype comprises two class I CFTR mutations. The subject's genotype may include any combination of class I CFTR mutations. As one non-limiting example, in some instances, the subject's genotype comprises a W1282X mutation and a R1162X mutation.

In other examples, the subject's genotype may comprise at least one class II CFTR mutation (e.g., F508del, N1303K, or A561E), at least one class III CFTR mutation (e.g., G551 D, S549R, or G1349D), at least one class IV CFTR mutation (e.g., R117H, R334W, or A455E), at least one class V CFTR mutation (e.g., A455G, 3272-26A→G, or 3849+10 kg C→T), at least one class VI CFTR mutation (e.g., dele2,3(21 kb), 1717-1 G→A), or at least one class VII CFTR mutation (e.g., dele2,3(21 kb) and 1717-1 G→A). See, e.g., De Boeck et al. Acta Paediatrica 2020; 109:893-899, for a review of different classes of CFTR mutations.

For example, in any of the preceding examples in which an augmenter is administered to a subject following administration of an rAAV, the subject's genotype may comprise at least one class II CFTR mutation (e.g., F508del, N1303K, or A561E). In some examples, the subject's genotype does not comprise an F508del mutation. For example, in some examples, the subject's genotype includes a class II CFTR mutation that does not comprise an F580del mutation. In some examples, the subject's genotype comprises two class II CFTR mutations. The subject's genotype may include any combination of class II CFTR mutations, including any combination of the class II CFTR mutations listed above.

In another example, in any of the preceding examples in which an augmenter is administered to a subject following administration of an rAAV, the subject's genotype may comprise at least one class III CFTR mutation (e.g., G551 D, S549R, or G1349D). In some examples, the subject's genotype comprises two class III CFTR mutations. The subject's genotype may include any combination of class Ill CFTR mutations, including any combination of the class III CFTR mutations listed above.

In another example, in any of the preceding examples in which an augmenter is administered to a subject following administration of an rAAV, the subject's genotype may comprise at least one class IV CFTR mutation (e.g., R117H, R334W, or A455E). In some examples, the subject's genotype comprises two class IV CFTR mutations. The subject's genotype may include any combination of class IV CFTR mutations, including any combination of the class IV CFTR mutations listed above.

In another example, in any of the preceding examples in which an augmenter is administered to a subject following administration of an rAAV, the subject's genotype may comprise at least one class V CFTR mutation (e.g., A455G, 3272-26A→G, or 3849+10 kg C→T). In some examples, the subject's genotype comprises two class V CFTR mutations. The subject's genotype may include any combination of class V CFTR mutations, including any combination of the class V CFTR mutations listed above.

In another example, in any of the preceding examples in which an augmenter is administered to a subject following administration of an rAAV, the subject's genotype may comprise at least one class VI CFTR mutation (e.g., dele2,3(21 kb), 1717-1 G→A). In some examples, the subject's genotype comprises two class VI CFTR mutations. The subject's genotype may include any combination of class VI CFTR mutations, including any combination of the class VI CFTR mutations listed above.

In another example, in any of the preceding examples in which an augmenter is administered to a subject following administration of an rAAV, the subject's genotype may comprise at least one class VII CFTR mutation (e.g., dele2,3(21 kb) and 1717-1 G→A). In some examples, the subject's genotype comprises two class VII CFTR mutations. The subject's genotype may include any combination of class VII CFTR mutations, including any combination of the class VII CFTR mutations listed above.

In some examples, the subject's genotype may comprise at least one of the following CFTR mutations: 185+1G>T, 296+1G>A, 296+1G>T, 405+1G>A, 405+3A>C, 406-1G>A, 621+1G>T, 711+1G>T, 711+5G>A, 712-1G>T, 1248+1G>A, 1249-1G>A, 1341+1G>A, 1525-2A>G, 1525-1G>A, 1717-8G>A, 1717-1 G>A, 1811+1 G>C, 1811+1.6kbA>G, 1811+1643G>T, 1812-1 G>A, 1898+1 G>A, 1898+1G>C, 2622+1G>A, 2790-1G>C, 3040G>C (G970R), 3120G>A, 3120+1G>A, 3121-2A>G, 3121-1G>A, 3500-2A>G, 3600+2insT, 3850-1G>A, 4005+1G>A, 4374+1G>T, 182delT, 306insA, 365-366insT, 394delTT, 442delA, 444delA, 457TAT>G, 541 delC, 574delA, 663delT, 849delG, 935delA, 1078delT, 1119delA, 1138insG, 1154insTC, 1161delC, 1213delT, 1259insA, 1288insTA, 1343delG, 1471delA, 1497delGG, 1548delG, 1609del CA, 1677delTA, 1782delA, 1824delA, 1833delT, 2043delG, 2143delT, 2183AA>G^{a}, 2184delA, 2184insA, 2307insA, 2347delG, 2585delT, 2594delGT, 2711delT, 2732insA, 2869insG, 2896insAG, 2942insT, 2957delT, 3007delG, 3028delA, 3171delC, 3171insC, 3271delGG, 3349insT, 3659delC, 3737delA, 3791delC, 3821delT, 3876delA, 3878delG, 3905insT, 4016insT, 4021dupT, 4022insT, 4040delA, 4279insA, 4326delTC, CFTRdele1, CFTRdele2, CFTRdele2,3, CFTRdele2-4, CFTRdele3-10,14b-16, CFTRdele4-7, CFTRdele4-11, CFTR50kbdel, CFTRdup6b-10, CFTRdele11, CFTRdele13,14a, CFTRdele14b-17b, CFTRdele16-17b, CFTRdele17a,17b, CFTRdele17a-18, CFTRdele19, CFTRdele19-21, CFTRdele21, CFTRdele22-24, CFTRdele22,23, 124del23 bp, 306delTAGA, 602del14, 852del22, 991del5, 1461 ins4, 1924de17, 2055de19>A, 2105-2117del13insAGAAA, 2372del8, 2721 dell 1, 2991del32, 3121-977_3499+248del2515, 3667ins4, 4010del4, 4209TGTT>AA, A46D, G85E, R347P, L467P, 1507del, V520F, A559T, R560T, R560S, A561E, Y569D, L1065P, R1066C, L1077P, M1101K, and N1303K.

In some examples, the rAAV comprises an AV.TL65 capsid protein. In some examples, the AV.TL65 capsid protein comprises the amino acid sequence of SEQ ID NO:13 or a sequence having at least 80%, at least 85%, at least 90%, at least 91%, at least 92%, at least 93%, at least 94%, at least 95%, at least 96%, at least 97%, at least 98%, at least 99%, or more, identical to SEQ ID NO:13. In some embodiments, the sequence having at least 80%, at least 85%, at least 90%, at least 91%, at least 92%, at least 93%, at least 94%, at least 95%, at least 96%, at least 97%, at least 98%, at least 99%, or more, identical to SEQ ID NO:13 comprises a Threonine (T) residue at position 581. In some examples, the AV.TL65 capsid protein comprises the amino acid sequence of SEQ ID NO:13.

The rAAV may include any of the polynucleotides described herein.

In some examples, the polynucleotide comprises an F5 enhancer. In some examples, the F5 enhancer comprises the polynucleotide sequence of SEQ ID NO:1. In other examples, the F5 enhancer comprises the polynucleotide sequence of SEQ ID NO:14.

In some examples, the polynucleotide comprises a tg83 promoter. In some examples, the tg83 promoter comprises the polynucleotide sequence of SEQ ID NO:2.

In some examples, the polynucleotide comprises a CFTRΔR minigene. In some examples, the CFTRΔR minigene is a human CFTRΔR minigene. In some examples, the human CFTRΔR minigene is encoded by a polynucleotide comprising the sequence of SEQ ID NO:4.

In some examples, the polynucleotide comprises, in a 5′-to-3′ direction, the F5 enhancer, the tg83 promoter, and the CFTRΔR minigene.

In some examples, the polynucleotide comprises the sequence of SEQ ID NO:7.

In some examples, the method further comprises administering one or more additional therapeutic agents to the subject. Any suitable additional therapeutic agent(s) or combination thereof may be used, e.g., any additional therapeutic agent(s) disclosed herein. In some examples, the one or more additional therapeutic agents includes an antibiotic, a mucus thinner, a CFTR modulator, a mucolytic, normal saline, hypertonic saline, an immunosuppressive agent, or a combination thereof.

In accordance with the methods disclosed herein, a composition described herein (e.g., an rAAV or pharmaceutical composition) may be used in vivo as well as ex vivo. In vivo gene therapy comprises administering the vectors of this disclosure directly to a subject. Pharmaceutical compositions can be supplied as liquid solutions or suspensions, as emulsions, or as solid forms suitable for dissolution or suspension in liquid prior to use. For administration into the respiratory tract, one exemplary mode of administration is by aerosol, using a composition that provides either a solid or liquid aerosol when used with an appropriate aerosolubilizer device. Another mode of administration into the respiratory tract is using a flexible fiberoptic bronchoscope to instill the vectors.

In accordance with the methods disclosed herein, a composition described herein (e.g., an rAAV or pharmaceutical composition) can be administered by any suitable route, e.g., by inhalation, nebulization, aerosolization, intranasally, intratracheally, intrabronchially, orally, parenterally (e.g., intravenously, subcutaneously, or intramuscularly), orally, nasally, rectally, topically, or buccally. They can also be administered locally or systemically. In some embodiments, a composition described herein is administered in aerosolized particles intratracheally and/or intrabronchially using an atomizer sprayer (e.g., with a MADgic® laryngo-tracheal mucosal atomization device). In some embodiments, the composition is administered parentally. In other some embodiments, the composition is administered systemically. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions are within the skill of the art.

For administration to the upper (nasal) or lower respiratory tract by inhalation, the compositions described herein (e.g., rAAVs or pharmaceutical compositions) are conveniently delivered from an insufflator, nebulizer or a pressurized pack or other convenient means of delivering an aerosol spray.

Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the agent may be administered via nose drops, a liquid spray, such as via a plastic bottle atomizer or metered-dose inhaler. Typical of atomizers are the Mistometer (Wintrop) and the Medihaler (Riker).

In accordance with the methods disclosed herein, a composition described herein (e.g., an rAAV or pharmaceutical composition) may be continuous or intermittent, depending, for example, upon the recipient's physiological condition, whether the purpose of the administration is therapeutic or prophylactic, and other factors known to skilled practitioners. The compositions described herein can be administered once, or multiple times (e.g., 2, 3, 4, 5, 6, 7, 8, 9, 10, 20, or more times), at the same or at different sites. The administration of the agents of the disclosure may be essentially continuous over a preselected period of time or may be in a series of spaced doses.

In accordance with the methods disclosed herein, a composition described herein (e.g., an rAAV or pharmaceutical composition) may be administered as a monotherapy. The compositions described herein (e.g., rAAVs or pharmaceutical compositions) can also be administered in combination with one or more additional therapeutic agent. Any suitable additional therapeutic agent(s) may be used, including standard of care therapies for CF. In some embodiments, the one or more additional therapeutic agents includes an antibiotic (e.g., azithromycin (ZITHROMAX®), amoxicillin and clavulanic acid (AUGMENTIN®), cloxacillin and diclocacillin, ticarcillin and clavulanic acid (TIMENTIN®), cephalexin, cefdinir, cefprozil, cefaclor; sulfamethoxazole and trimethoprim (BACTRIM®), erythromycin/sulfisoxazole, erythromycin, clarithromycin, tetracycline, doxycycline, minocycline, tigecycline, vancomycin, imipenem, meripenem, Colistimethate/COLISTIN®, linezolid, ciprofloxacin, levofloxacin, or a combination thereof), a mucus thinner (e.g., hypertonic saline or dornase alfa (PULMOZYME®)), a CFTR modulator (e.g., ivacaftor (KALYDECO®), lumacaftor, lumacaftor/ivacaftor (ORKAMBI®), tezacaftor/ivacaftor (SYMDEKO®), or TRIKAFTA® (elexacaftor/ivacaftor/tezacaftor)), a mucolytic (e.g., acetylcysteine, ambroxol, bromhexine, carbocisteine, erdosteine, mecysteine, and dornase alfa), an immunosuppressive agent, normal saline, hypertonic saline, or a combination thereof.

For example, any of the methods disclosed herein may include administering any one the compositions described herein (e.g., rAAVs or pharmaceutical compositions) in combination with one or more immunosuppressive agents. Any suitable immunosuppressive agent may be used. For example, non-limiting examples of immunosuppressive agents include corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), predisone, or methylprednisone), polyclonal anti-lymphocyte antibodies (e.g., anti-lymphocyte globulin (ALG) and anti-thymocyte globulin (ATG) antibodies, which may be, for example, horse- or rabbit-derived), monoclonal anti-lymphocyte antibodies (e.g., anti-CD3 antibodies (e.g., murmomab and alemtuzumab) or anti-CD20 antibodies (e.g., rituximab)), interleukin-2 (IL-2) receptor antagonists (e.g., daclizumab and basiliximab), calcineurin inhibitors (e.g., cyclosporin A and tacrolimus), cell cycle inhibitors (e.g., azathioprine, mycophenolate mofetil (MMF), and mycophenolic acid (MPA)), mammalian target of rapamycin (mTOR) inhibitors (e.g., sirolimus (rapamycin) and everolimus), methotrexate, cyclophosphamide, an anthracycline (e.g., doxorubicin, idarubicin, aclarubicin, daunorubicin, epirubicin, valrubicin, mitoxantrone, or a combination thereof), a taxane (e.g., TAXOL® (paclitaxel)), and a combination thereof (e.g., a combination of a calcineurin inhibitor, a cell cycle inhibitor, and a corticosteroid).

In particular embodiments, any of the methods disclosed herein may include administering any one the compositions described herein (e.g., rAAVs or pharmaceutical compositions) in combination with one or more corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), predisone, or methylprednisone). In some embodiments, the corticosteroid is an inhaled corticosteroid.

An immunosuppressive agent (e.g., any immunosuppressive agent described herein) may be administered by inhalation or administered systemically (e.g., intravenously or subcutaneously).

In some examples, any of the methods disclosed herein may include administering any one the compositions described herein (e.g., rAAVs or pharmaceutical compositions) to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the present compositions will vary with the form of administration, the particular compound chosen and the physiological characteristics of the particular patient under treatment. It is desirable that the lowest effective concentration of virus be utilized in order to reduce the risk of undesirable effects, such as toxicity.

### Polynucleotides

The disclosure provides polynucleotides which may be incorporated into rAAV vectors for use in the methods disclosed herein, or used in the preparation of rAAV vectors. The polynucleotide may include any suitable elements or components, including one or more elements selected from a 5′ AAV ITR (e.g., an AAV2 5′ ITR), an F5 enhancer, a tg83 promoter, a 5′ untranslated region (UTR), a CFTRΔR minigene, a 3′ UTR, a polyadenylation site, and/or a 3′ AAV ITR (e.g., an AAV2 3′ ITR). Although the polynucleotides are generally incorporated into rAAV vectors, it is to be understood that they could be delivered or administered in the context of other types of vectors that are known in the art. Any of the polynucleotides described below may be used in the methods disclosed herein.

In one aspect, the disclosure provides an isolated polynucleotide that includes the sequence of SEQ ID NO:7, or a sequence having at least 80%, 85%, 90%, 91%, 92%, 93%, 94%, 95%, 96%, 97%, 98%, 99% sequence identity with the polynucleotide sequence of SEQ ID NO:7. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In some embodiments, the polynucleotide further comprises, in the 3′ direction, a 3′ untranslated region (3′-UTR) comprising the sequence of SEQ ID NO:5, or a sequence having at least 80%, 85%, 90%, 91%, 92%, 93%, 94%, 95%, 96%, 97%, 98%, 99% sequence identity with the polynucleotide sequence of SEQ ID NO:5.

In some embodiments, the polynucleotide further comprises, in the 3′ direction (e.g., 3′ relative to the 3′-UTR), a synthetic polyadenylation site comprising the sequence of SEQ ID NO:6.

In some embodiments, the polynucleotide further comprises a 5′ adeno-associated virus (AAV) inverted terminal repeat (ITR) at the 5′ terminus of the polynucleotide and/or a 3′ AAV ITR at the 3′ terminus of the polynucleotide. In some embodiments, the polynucleotide comprises the sequence of SEQ ID NO:11, or a sequence having at least 80%, 85%, 90%, 91%, 92%, 93%, 94%, 95%, 96%, 97%, 98%, 99% sequence identity with the polynucleotide sequence of SEQ ID NO:11. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In other embodiments, the polynucleotide comprises the sequence of SEQ ID NO:17, or a sequence having at least 80%, 85%, 90%, 91%, 92%, 93%, 94%, 95%, 96%, 97%, 98%, 99% sequence identity with the polynucleotide sequence of SEQ ID NO:17. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

Any of the polynucleotides may contain a 5′ AAV ITR. Any suitable 5′ AAV ITR may be used, including a 5′ AAV ITR from any AAV serotype (e.g., AAV2). In some embodiments, the 5′ AAV ITR comprises the sequence of SEQ ID NO:9, or a sequence having at least 80%, 85%, 90%, 91%, 92%, 93%, 94%, 95%, 96%, 97%, 98%, 99% sequence identity with the polynucleotide sequence of SEQ ID NO:9. In another example, in some embodiments, the polynucleotide includes a 5′ AAV ITR comprising the sequence of SEQ ID NO:15, or a sequence having at least 80%, 85%, 90%, 91%, 92%, 93%, 94%, 95%, 96%, 97%, 98%, 99% sequence identity with the polynucleotide sequence of SEQ ID NO:15. Any of the polynucleotides may contain a 3′ AAV ITR. Any suitable 3′ AAV ITR may be used, including a 3′ AAV ITR from any AAV serotype (e.g., AAV2). In some embodiments, the 3′ AAV ITR comprises the sequence of SEQ ID NO:10, or a sequence having at least 80%, 85%, 90%, 91%, 92%, 93%, 94%, 95%, 96%, 97%, 98%, 99% sequence identity with the polynucleotide sequence of SEQ ID NO:10. In another example, in some embodiments, the polynucleotide includes a 3′ AAV ITR comprising the sequence of SEQ ID NO:16, or a sequence having at least 80%, 85%, 90%, 91%, 92%, 93%, 94%, 95%, 96%, 97%, 98%, 99% sequence identity with the polynucleotide sequence of SEQ ID NO:16. The ITR sequences may be palindromic, e.g., as in SEQ ID NO:15 and SEQ ID NO:16, where the ITR sequence on the 5′ end is located on the reverse strand, and the ITR sequence on the 3′ end is located on the forward strand.

Any of the polynucleotides may contain an F5 enhancer. See, e.g., U.S. patent application Ser. No. 16/082,767, which is incorporated herein by reference in its entirety. In some embodiments, the F5 enhancer comprises the sequence of SEQ ID NO:1 or SEQ ID NO:14, or a sequence having at least 80%, 85%, 90%, 91%, 92%, 93%, 94%, 95%, 96%, 97%, 98%, 99% sequence identity with the polynucleotide sequence of SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14.

Any of the polynucleotides may contain a tg83 promoter. See, e.g., U.S. patent application Ser. No. 16/082,767. In some embodiments, the tg83 promoter comprises the sequence of SEQ ID NO:2, or a sequence having at least 80%, 85%, 90%, 91%, 92%, 93%, 94%, 95%, 96%, 97%, 98%, 99% sequence identity with the polynucleotide sequence of SEQ ID NO:2.

Any of the polynucleotides may contain a 5′-UTR. Any suitable 5′-UTR may be used. In some embodiments, the 5′-UTR comprises the sequence of SEQ ID NO:3, or a sequence having at least 80%, 85%, 90%, 91%, 92%, 93%, 94%, 95%, 96%, 97%, 98%, 99% sequence identity with the polynucleotide sequence of SEQ ID NO:3.

Any of the polynucleotides may contain a sequence encoding a CFTRΔR minigene. Any suitable CFTRΔR minigene may be used, including human CFTRΔR (hCFTRΔR) or ferret CFTRΔR. In some embodiments, the sequence encoding an hCFTRΔR minigene comprises the sequence of SEQ ID NO:4, or a sequence having at least 80%, 85%, 90%, 91%, 92%, 93%, 94%, 95%, 96%, 97%, 98%, 99% sequence identity with the polynucleotide sequence of SEQ ID NO:4.

Any of the polynucleotides may contain a 3′-UTR. Any suitable 3′-UTR may be used. In some embodiments, the 3′-UTR comprises the sequence of SEQ ID NO:3, or a sequence having at least 80%, 85%, 90%, 91%, 92%, 93%, 94%, 95%, 96%, 97%, 98%, 99% sequence identity with the polynucleotide sequence of SEQ ID NO:5.

Any of the polynucleotides may contain a polyadenylation site. Any suitable polyadenylation site may be used. In some embodiments, the polyadenylation site comprises the sequence of SEQ ID NO:6, or a sequence having at least 80%, 85%, 90%, 91%, 92%, 93%, 94%, 95%, 96%, 97%, 98%, 99% sequence identity with the polynucleotide sequence of SEQ ID NO:6.

In one aspect, the disclosure provides an isolated polynucleotide that includes the sequence of SEQ ID NO:8, or a sequence having at least 80%, 85%, 90%, 91%, 92%, 93%, 94%, 95%, 96%, 97%, 98%, 99% sequence identity with the polynucleotide sequence of SEQ ID NO:8. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In one aspect, the disclosure provides an isolated polynucleotide that includes the sequence of SEQ ID NO:11, or a sequence having at least 80%, 85%, 90%, 91%, 92%, 93%, 94%, 95%, 96%, 97%, 98%, 99% sequence identity with the polynucleotide sequence of SEQ ID NO:11. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In one aspect, the disclosure provides an isolated polynucleotide that includes the sequence of SEQ ID NO:12, or a sequence having at least 80%, 85%, 90%, 91%, 92%, 93%, 94%, 95%, 96%, 97%, 98%, 99% sequence identity with the polynucleotide sequence of SEQ ID NO:12. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

In another aspect, the disclosure provides an isolated polynucleotide that includes the sequence of SEQ ID NO:18, or a sequence having at least 80%, 85%, 90%, 91%, 92%, 93%, 94%, 95%, 96%, 97%, 98%, 99% sequence identity with the polynucleotide sequence of SEQ ID NO:18. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

The polynucleotide may also contain one or more detectable markers. A variety of such markers are known, including, by way of illustration, the bacterial beta-galactosidase (lacZ) gene; the human placental alkaline phosphatase (AP) gene and genes encoding various cellular surface markers which have been used as reporter molecules both in vitro and in vivo. The polynucleotide may also contain one or more selectable markers.

### Recombinant AAV Vectors

Recombinant AAV vectors are potentially powerful tools for human gene therapy, particularly for diseases such as cystic fibrosis. A major advantage of rAAV vectors over other approaches to gene therapy is that they generally do not require ongoing replication of the target cell in order to exist episomally or become stably integrated into the host cell. In general, the disclosure provides an rAAV that includes an AV.TL65 capsid protein and a polynucleotide that includes an F5 enhancer and a tg83 promoter operably linked to a transgene. Any of the rAAVs described below may be used in the methods disclosed herein. In some examples, any rAAV disclosed in International Patent Application Publication No. WO 2020/214668 or in U.S. patent application Ser. No. 17,603/831, which are incorporated herein by reference in its entirety, may be used in the methods disclosed herein.

For example, in one aspect, the disclosure provides an rAAV that includes (i) an AV.TL65 capsid protein; and (ii) a polynucleotide including an F5 enhancer and a tg83 promoter operably linked to a CFTRΔR minigene.

In another aspect, the disclosure provides an rAAV for use in treating cystic fibrosis (e.g., CF associated with a class I mutation) in a subject in need thereof, the rAAV including (i) an AV.TL65 capsid protein; and (ii) a polynucleotide including an F5 enhancer and a tg83 promoter operably linked to a CFTRΔR minigene.

In some embodiments, the AV.TL65 capsid protein includes the amino acid sequence of SEQ ID NO:13, or an amino acid sequence having at least 80%, 85%, 90%, 91%, 92%, 93%, 94%, 95%, 96%, 97%, 98%, 99% sequence identity with the amino acid sequence of SEQ ID NO:13. In some embodiments, the sequence having at least 80%, at least 85%, at least 90%, at least 91%, at least 92%, at least 93%, at least 94%, at least 95%, at least 96%, at least 97%, at least 98%, at least 99%, or more, identical to SEQ ID NO:13 comprises a Threonine (T) residue at position 581.

In some embodiments, the F5 enhancer includes the polynucleotide sequence of SEQ ID NO:1 or SEQ ID NO:14, or a sequence having at least 80%, 85%, 90%, 91%, 92%, 93%, 94%, 95%, 96%, 97%, 98%, 99% sequence identity with the polynucleotide sequence of SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14.

In some embodiments, the tg83 promoter includes the polynucleotide sequence of SEQ ID NO:2, or a sequence having at least 80%, 85%, 90%, 91%, 92%, 93%, 94%, 95%, 96%, 97%, 98%, 99% sequence identity with the polynucleotide sequence of SEQ ID NO:2.

Any suitable CFTRΔR minigene or a derivative thereof may be used. In some embodiments, the CFTRΔR minigene is a human CFTRΔR minigene. In other embodiments, the CFTRΔR minigene is a ferret CFTRΔR minigene. In some embodiments, the human CFTRΔR minigene is encoded by a polynucleotide including the sequence of SEQ ID NO:4, or a sequence having at least 80%, 85%, 90%, 91%, 92%, 93%, 94%, 95%, 96%, 97%, 98%, 99% sequence identity with the polynucleotide sequence of SEQ ID NO:4.

In some embodiments, the polynucleotide includes, in a 5′-to-3′ direction, the F5 enhancer, the tg83 promoter, and the CFTRΔR minigene. In some particular embodiments, the polynucleotide comprises, in a 5′-to-3′ direction, a 5′ AAV ITR (e.g., an AAV2 5′ ITR), the F5 enhancer, the tg83 promoter, a 5′ untranslated region (UTR), the CFTRΔR minigene, a '3-UTR, a polyadenylation site, and a 3′ AAV ITR (e.g., an AAV2 3′ ITR).

In another aspect, the disclosure provides an rAAV comprising any of the polynucleotides described herein, e.g., a polynucleotide comprising the sequence of SEQ ID NO:7, SEQ ID NO:11, or SEQ ID NO:17, or a sequence having at least 80%, 85%, 90%, 91%, 92%, 93%, 94%, 95%, 96%, 97%, 98%, 99% sequence identity with the polynucleotide sequence of SEQ ID NO:7, SEQ ID NO:11, or SEQ ID NO:17. For example, the disclosure provides an rAAV comprising a polynucleotide comprising the sequence of SEQ ID NO:17, or a sequence having at least 80%, 85%, 90%, 91%, 92%, 93%, 94%, 95%, 96%, 97%, 98%, 99% sequence identity with the polynucleotide sequence of SEQ ID NO:17. In some embodiments, the rAAV has a tropism for airway epithelial cells (e.g., lung epithelial cells). In some embodiments, the rAAV comprises an AV.TL65 capsid protein, an AAV1 capsid protein, an AAV2 capsid protein, an AAV5 capsid protein, an AAV6 capsid protein, or an AAV9 capsid protein. In some embodiments, the rAAV comprises an AV.TL65 capsid protein. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

The heterologous polynucleotide may be integrated by recombinant techniques into or preferably in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, preferably (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present disclosure.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters are some because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1 CP4; as well as vaccinia or poxvirus inducible promoters.

Given the relative encapsidation size limits of various AAV genomes, insertion of a large heterologous polynucleotide into the genome necessitates removal of a portion of the AAV sequence. Removal of one or more AAV genes is in any case desirable, to reduce the likelihood of generating replication-competent AAV (“RCA”). Accordingly, encoding or promoter sequences for rep, cap, or both, are preferably removed, since the functions provided by these genes can be provided in trans.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are preferably not flanked by AAV ITRs and preferably do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this disclosure in a number of different forms. Viral particles, plasmids, and stably transformed host cells can all be used to introduce such constructs into the packaging cell, either transiently or stably.

In certain embodiments of this disclosure, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this disclosure. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., (WO 95/13392); Burstein et al. (WO 98/23018); and Johnson et al. (U.S. Pat. No. 5,656,785)). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this disclosure.

Approaches for producing rAAVs, e.g., rAAVs that contain AV.TL65 capsid proteins are known in the art. See, e.g., Excoffon et al. Proc. Natl. Acad. Sci. USA 106(10):3865-3870, 2009 and U.S. Pat. No. 10,046,016, each of which is incorporated herein by reference in its entirety.

### Augmenters

Any of the methods disclosed herein may include administration of an augmenter of AAV transduction (also referred to as “augmenter”) to the subject. In some examples, the augmenter is administered to the subject following administration of an rAAV vector disclosed herein, e.g., within about 72 h, about 48 h, about 24 h, or about 12 hr. For example, the rAAVs described herein can be used in combination with augmenters of AAV transduction to achieve significant increases in transduction and/or expression of transgenes. Any suitable augmenter can be used. For example, U.S. Pat. No. 7,749,491, which is incorporated by reference herein in its entirety, describes suitable augmenters. The augmenter may be a proteasome modulating agent. The augmenter may be an anthracycline (e.g., doxorubicin, idarubicin, aclarubicin, daunorubicin, epirubicin, valrubicin, or mitoxantrone), a proteasome inhibitor (e.g., bortezomib, carfilzomib, and ixazomib), a tripeptidyl aldehyde (e.g., N-acetyl-1-leucyl-1-leucyl-1-norleucine (LLnL)), or a combination thereof. In some embodiments, the augmenter is doxorubicin, e.g., doxorubicin-HCl. In other embodiments, the augmenter is idarubicin.

The rAAV and the augmenter(s) may be contacted with a cell, or administered to a subject, in the same composition or in different compositions (e.g., pharmaceutical compositions). The contacting or the administration of the rAAV and the augmenter(s) may be sequential (e.g., rAAV followed by the augmenter(s), or vice versa) or simultaneous.

### Pharmaceutical Compositions

The disclosure provides pharmaceutical compositions for use in the methods disclosed herein, including pharmaceutical compositions that include any of the rAAVs described herein. The pharmaceutical carrier may include one or more pharmaceutically acceptable carriers, excipients, diluents, buffers, and the like. Any of the pharmaceutical compositions described below may be used in any of the methods disclosed herein.

For example, in one aspect, the disclosure provides a pharmaceutical composition that includes an rAAV, the rAAV including (i) an AV.TL65 capsid protein; and (ii) a polynucleotide including an F5 enhancer and a tg83 promoter operably linked to a CFTRΔR minigene.

In another aspect, the disclosure provides a pharmaceutical composition comprising an rAAV for use in treating cystic fibrosis in a subject in need thereof (e.g., CF associated with a class I mutation), the rAAV including (i) an AV.TL65 capsid protein; and (ii) a polynucleotide including an F5 enhancer and a tg83 promoter operably linked to a CFTRΔR minigene.

In some embodiments, the AV.TL65 capsid protein includes the amino acid sequence of SEQ ID NO:13, or an amino acid sequence having at least 80%, 85%, 90%, 91%, 92%, 93%, 94%, 95%, 96%, 97%, 98%, 99% sequence identity with the amino acid sequence of SEQ ID NO:13. In some embodiments, the sequence having at least 80%, at least 85%, at least 90%, at least 91%, at least 92%, at least 93%, at least 94%, at least 95%, at least 96%, at least 97%, at least 98%, at least 99%, or more, identical to SEQ ID NO:13 comprises a Threonine (T) residue at position 581.

In some embodiments, the F5 enhancer includes the polynucleotide sequence of SEQ ID NO:1 or SEQ ID NO:14, or a sequence having at least 80%, 85%, 90%, 91%, 92%, 93%, 94%, 95%, 96%, 97%, 98%, 99% sequence identity with the polynucleotide sequence of SEQ ID NO:1 or SEQ ID NO:14. In some embodiments, the F5 includes the polynucleotide sequence of SEQ ID NO:1. In other embodiments, the F5 enhancer includes the polynucleotide sequence of SEQ ID NO:14.

In some embodiments, the tg83 promoter includes the polynucleotide sequence of SEQ ID NO:2, or a sequence having at least 80%, 85%, 90%, 91%, 92%, 93%, 94%, 95%, 96%, 97%, 98%, 99% sequence identity with the polynucleotide sequence of SEQ ID NO:2.

Any suitable CFTRΔR minigene or a derivative thereof may be used. In some embodiments, the CFTRΔR minigene is a human CFTRΔR minigene. In other embodiments, the CFTRΔR minigene is a ferret CFTRΔR minigene. In some embodiments, the human CFTRΔR minigene is encoded by a polynucleotide including the sequence of SEQ ID NO:4, or a sequence having at least 80%, 85%, 90%, 91%, 92%, 93%, 94%, 95%, 96%, 97%, 98%, 99% sequence identity with the polynucleotide sequence of SEQ ID NO:4.

In some embodiments, the polynucleotide includes, in a 5′-to-3′ direction, the F5 enhancer, the tg83 promoter, and the CFTRΔR minigene. In some particular embodiments, the polynucleotide comprises, in a 5′-to-3′ direction, a 5′ AAV ITR (e.g., an AAV2 5′ ITR), the F5 enhancer, the tg83 promoter, a 5′ untranslated region (UTR), the CFTRΔR minigene, a 3′-UTR, a polyadenylation site, and a 3′ AAV ITR (e.g., an AAV2 3′ ITR).

In another aspect, the disclosure provides a pharmaceutical composition comprising an rAAV, the rAAV comprising any of the polynucleotides described herein, e.g., a polynucleotide comprising the sequence of SEQ ID NO:7, 11, or 17, or a sequence having at least 80%, 85%, 90%, 91%, 92%, 93%, 94%, 95%, 96%, 97%, 98%, 99% sequence identity with the polynucleotide sequence of SEQ ID NO:7, 11, or 17). For example, provided herein is a pharmaceutical composition comprising an rAAV, the rAAV comprising a polynucleotide comprising the sequence of SEQ ID NO:17, or a sequence having at least 80%, 85%, 90%, 91%, 92%, 93%, 94%, 95%, 96%, 97%, 98%, 99% sequence identity with the polynucleotide sequence of SEQ ID NO:17. In some embodiments, the rAAV has a tropism for airway epithelial cells (e.g., lung epithelial cells). In some embodiments, the rAAV comprises an AV.TL65 capsid protein, an AAV1 capsid protein, an AAV2 capsid protein, an AAV5 capsid protein, an AAV6 capsid protein, or an AAV9 capsid protein. In some embodiments, the rAAV comprises an AV.TL65 capsid protein. In some embodiments, the polynucleotide includes an F5 enhancer comprising the sequence of SEQ ID NO:1, a tg83 promoter comprising the sequence of SEQ ID NO:2, and/or a hCFTRΔR minigene comprising the sequence of SEQ ID NO:4. In another some embodiment, the polynucleotide includes an F5 enhancer comprising the sequence of SEQ ID NO:14, a tg83 promoter comprising the sequence of SEQ ID NO:2, and/or a hCFTRΔR minigene comprising the sequence of SEQ ID NO:4.

Also provided herein is a pharmaceutical composition comprising one or more augmenters. Any of the augmenters disclosed herein (e.g., doxorubicin) may be included in the pharmaceutical composition.

The pharmaceutical compositions described herein may include an rAAV alone, or an rAAV in combination with one or more additional therapeutic agents. Exemplary additional therapeutic agents include, without limitation, an antibiotic (e.g., azithromycin (ZITHROMAX®), amoxicillin and clavulanic acid (AUGMENTIN®), cloxacillin and diclocacillin, ticarcillin and clavulanic acid (TIMENTIN®), cephalexin, cefdinir, cefprozil, cefaclor; sulfamethoxazole and trimethoprim (BACTRIM®), erythromycin/sulfisoxazole, erythromycin, clarithromycin, tetracycline, doxycycline, minocycline, tigecycline, vancomycin, imipenem, meripenem, Colistimethate/COLISTIN®, linezolid, ciprofloxacin, levofloxacin, or a combination thereof), a mucus thinner (e.g., hypertonic saline or dornase alfa (PULMOZYME®)), a CFTR modulator (e.g., ivacaftor (KALYDECO®), lumacaftor, lumacaftor/ivacaftor (ORKAMBI®), tezacaftor/ivacaftor (SYMDEKO®), or TRIKAFTA® (elexacaftor/ivacaftor/tezacaftor)), a mucolytic (e.g., acetylcysteine, ambroxol, bromhexine, carbocisteine, erdosteine, mecysteine, and dornase alfa), an immunosuppressive agent, normal saline, hypertonic saline, or a combination thereof.

For example, pharmaceutical compositions described herein may include one or more immunosuppressive agents. Any suitable immunosuppressive agent may be used. For example, non-limiting examples of immunosuppressive agents include corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), predisone, or methylprednisone), polyclonal anti-lymphocyte antibodies (e.g., anti-lymphocyte globulin (ALG) and anti-thymocyte globulin (ATG) antibodies, which may be, for example, horse- or rabbit-derived), monoclonal anti-lymphocyte antibodies (e.g., anti-CD3 antibodies (e.g., murmomab and alemtuzumab) or anti-CD20 antibodies (e.g., rituximab)), interleukin-2 (IL-2) receptor antagonists (e.g., daclizumab and basiliximab), calcineurin inhibitors (e.g., cyclosporin A and tacrolimus), cell cycle inhibitors (e.g., azathioprine, mycophenolate mofetil (MMF), and mycophenolic acid (MPA)), mammalian target of rapamycin (mTOR) inhibitors (e.g., sirolimus (rapamycin) and everolimus), methotrexate, cyclophosphamide, an anthracycline (e.g., doxorubicin, idarubicin, aclarubicin, daunorubicin, epirubicin, valrubicin, mitoxantrone, or a combination thereof), a taxane (e.g., TAXOL® (paclitaxel)), and a combination thereof (e.g., a combination of a calcineurin inhibitor, a cell cycle inhibitor, and a corticosteroid).

In particular embodiments, pharmaceutical compositions described herein may include an one or more corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), predisone, or methylprednisone). In some embodiments, the corticosteroid is an inhaled corticosteroid.

An immunosuppressive agent (e.g., any immunosuppressive agent described herein) may be administered by inhalation or administered systemically (e.g., intravenously or subcutaneously).

Typically, the viral vectors are in a pharmaceutically suitable pyrogen-free buffer such as Ringer's balanced salt solution (pH 7.4). Although not required, pharmaceutical compositions may optionally be supplied in unit dosage form suitable for administration of a precise amount. Pharmaceutical compositions are generally sterile.

Kits and Articles of Manufacture

Provided herein are kits and articles of manufacture that may be used to perform the methods disclosed herein. For example, the kit or article of manufacture may include one or more of an rAAV (e.g., AV.TL65-SP183-hCFTRΔR), an augmenter (e.g., doxorubicin), and instructions to administer the rAAV and/or the augmenter to a subject having CF in accordance with any of the methods disclosed herein.

In one aspect, the disclosure provides a kit for treating CF in a subject whose genotype comprises at least one class I CFTR mutation, the kit comprising a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject may have any class I CFTR mutation. In some examples, the at least one class I CFTR mutation is a nonsense mutation, a splice mutation, or a deletion. In some examples, the at least one class I CFTR mutation comprises a Q2X mutation, a S4X mutation, a W19X mutation, a G27X mutation, a Q39X mutation, a W57X mutation, a E60X mutation, a R75X mutation, a L88X mutation, a E92X mutation, a Q98X mutation, a Y122X mutation, a E193X mutation, a W216X mutation, a L218X mutation, a Q220X mutation, a Y275X mutation, a C276X mutation, a Q290X mutation, a G330X mutation, a W401X mutation, a Q414X mutation, a S434X mutation, a S466X mutation, a S489X mutation, a Q493X mutation, a W496X mutation, a C524X mutation, a Q525X mutation, a G542X mutation, a G550X mutation, a Q552X mutation, a R553X mutation, a E585X mutation, a G673X mutation, a Q685X mutation, a R709X mutation, a K71 OX mutation, a Q715X mutation, a L732X mutation, a R764X mutation, a R785X mutation, a R792X mutation, a E822X mutation, a W882X mutation, a W846X mutation, a Y849X mutation, a R851X mutation, a Q890X mutation, a S912X mutation, a Y913X mutation, a Q1042X mutation, a W1089X mutation, a Y1092X mutation, a W1098X mutation, a R1102X mutation, a E 1104X mutation, a W1145X mutation, a R1158X mutation, a R1162X mutation, a S1196X mutation, a W1204X mutation, a L1254X mutation, a S1255X mutation, a W1282X mutation, a Q1313X mutation, a Q1330X mutation, a E1371X mutation, a Q1382X mutation, a Q1411X mutation, a 2116delCTAA mutation, or a combination thereof. In some examples, the at least one class I CFTR mutation comprises a G542X mutation, a W1282X mutation, an R1162X mutation, an R553X mutation, or a combination thereof. Other class I CFTR mutations are known in the art.

In some examples, the subject's genotype comprises two class I CFTR mutations. The subject's genotype may include any combination of class I CFTR mutations. As one non-limiting example, in some instances, the subject's genotype comprises a W1282X mutation and a R1162X mutation.

In one aspect, the disclosure provides a kit for treating CF in a subject whose genotype comprises at least one class II CFTR mutation, the kit comprising a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a kit for treating CF in a subject whose genotype comprises at least one class III CFTR mutation, the kit comprising a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

The subject's genotype may include any class Ill CFTR mutation. Exemplary class III CFTR mutations include, e.g., G551 D and S549N.

In some examples, the subject's genotype comprises two class III CFTR mutations. The subject's genotype may include any combination of class Ill CFTR mutations, including any combination of the class III CFTR mutations listed above.

In one aspect, the disclosure provides a kit for treating CF in a subject whose genotype comprises at least one class IV CFTR mutation, the kit comprising a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a kit for treating CF in a subject whose genotype comprises at least one class V CFTR mutation, the kit comprising a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a kit for treating CF in a subject whose genotype comprises at least one class VI CFTR mutation, the kit comprising a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In one aspect, the disclosure provides a kit for treating CF in a subject whose genotype comprises at least one class VII CFTR mutation, the kit comprising a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

In some examples, the kit further comprises an augmenter of AAV transduction, e.g., any augmenter described herein.

In some examples, the kit includes instructions to administer the augmenter to the subject within about 72 h (e.g., within about 48 h, within about 24 h, or within about 12 h) following administration of the rAAV.

In another aspect, the disclosure provides a kit for treating CF in a subject, the kit comprising: an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and instructions to administer to the subject an augmenter of AAV transduction within about 72 h following administration of the rAAV.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Delivery of SP-101 Restores CFTR Function in Human CF Airway Epithelial Cultures, Including from Class I Genotypes, and Drives hCFTRΔR Transgene Expression in the Airways of CF Ferrets

SP-101 (AV.TL65-SP183-hCFTRΔR) is a novel recombinant adeno-associated virus (AAV) gene therapy vector which may be administered by inhalation to people with cystic fibrosis (CF) who do not benefit from treatment with small molecule modulators. This population represents ˜20% of all people with CF. For example, patients with class I genotypes, which result in no functional CFTR protein being produced, do not benefit from correctors such as lumacaftor or tezacaftor.

SP-101 comprises the AV.TL65 capsid protein which is optimized for efficient apical transduction of human airway epithelial (HAE) cultures and a synthetic promoter enhancer sequence (SP-183 or F5tg83) driving the expression of a human CFTR minigene (hCFTRΔR) that is functionally equivalent to full-length CFTR.

When delivered to the apical surface of CF HAE cultures of various class I or II genotypes, SP-101 restored forskolin-induced CFTR-mediated chloride conductance to similar levels as those observed for modulator treatment. Chloride conductance increased with increasing multiplicity of infection and was strongly enhanced by co-administration of a small molecule, doxorubicin.

Tropism of the AV.TL65 capsid for ferret airway epithelial cultures has been shown with a reporter (AAV2.5T-CBAmCherry-SP183gLuc) and CF ferrets develop spontaneous lung disease resembling many features of CF. To investigate the potential of SP-101 to drive expression of hCTRΔR in the airways, SP-101 was delivered to wild-type and CF ferrets via nose-only inhalation using a nebulizer attached to a plenum exposure system, followed by nebulized doxorubicin, within 24 h. hCFTRΔR mRNA expression was determined by RT-qPCR, optimized for removal of viral genome DNA, and normalized to total RNA. Strikingly, robust hCFTRΔR expression was observed in the respiratory tract of both wild type and CF ferrets. Levels of hCFTRΔR expression increased with increasing doses of SP-101/doxorubicin and were highest in lung tissues. Expression was evident as early as 48 h post-exposure, lasting for at least 12 weeks, the latest timepoint investigated. Importantly, comparable hCFTRΔR mRNA expression was evident in the respiratory tract of CF ferrets indicating successful airway transduction despite pre-existing mucus accumulation.

In view of these data, it is expected that SP-101 will be effective for treatment of CF patients, including CF patients with class I genotypes that do not benefit from currently-available CF therapies.

### Example 2: Time Course of the Effect of Doxorubicin Addition on Transduction with a Reporter Vector (AV.TL65-CBA-mCHerry-SP183) in Primary Human Airway Epithelia

In this study, the time course associated with augmentation of AAV transduction by doxorubicin was explored using the reporter viral construct AV.TL65-CBA-mCherry-SP183. The experimental model was an in vitro assay based on polarized air-liquid interface (ALI) human airway epithelia (HAE).

The recombinant AAV (rAAV) reporter construct AV.TL65-CBA-mCherry-SP183 incorporates the same capsid as SP-101 (AV.TL65) which is highly tropic for the apical side of human airway epithelium, the side facing the lumen of the airways. In addition, this rAAV reporter construct uses the promoter F5tg83 to drive Gaussia luciferase expression; F5tg83 (also referenced as SP183) is the same promoter that drives the expression of the human hCFTRΔR transgene in SP-101.

Based on the shared characteristics summarized above, the rAAV reporter AV.TL65-CBA-mCherry-SP183 can be considered a surrogate for SP-101 transduction for in vitro mechanistic studies.

The transduction efficacy with AV.TL65-CBA-mCherry-SP183 was examined in an in vitro assay that measures functional luciferase secreted into the culture media of polarized human airway epithelial cell cultures. Different time-points of treatment with the augmenter doxorubicin post-transduction were studied to determine an optimized timing of treatment to enhance AAV transduction. A 2-hour treatment with doxorubicin at various time-points within 40 hours post-transduction resulted in similar efficacy, establishing the time window for optimal transduction efficacy driven by the augmenter doxorubicin in this type of studies. A trend for slightly lower luciferase signal was observed when doxorubicin was administered >40 hours post AAV.

Methods and Materials

Study Design

The objective of this study was to determine an optimized time window of treatment with the augmenter doxorubicin to enhance the transduction activity of the reporter AV.TL65-CBA-mCherry-SP183 in polarized HAE. The endpoint assessment of the efficiency of transduction with the reporter AAV was the level of luciferase activity detected in the conditioned media of transduced cells treated with doxorubicin for 2 hours at different time-points post-AAV exposure. The effect of doxorubicin treatment was explored at different time-points between 2 and 88 hours post-AAV exposure in a series of independent experiments. Luciferase activity was determined at 2, 4, 6, or 8 Days After Transduction (DAT) in the different studies summarized in this Example.

Production of rAAV

Viral integrity was verified using methods described previously (Yan et al. J. Virol. 78:2863-2874, 2004; Yan et al. Hum. Gene Ther. 26:334-346, 2015). Briefly, plasmid was produced using the Escherichia coli SURE strain (Stratagene) and isolated using an endotoxin-free plasmid isolation kit. Recombinant AAV was produced by co-transfection of human embryonic kidney 293 cells with three plasmids and was purified by two rounds of CsCl ultracentrifugation. TaqMan® quantitative reverse transcription polymerase chain reaction was used to quantify the physical titer (DNase-resistant particles (DRP) of the purified viral stocks, as described in Ding et al. Mol. Ther. 13:671-682, 2006.

Cell Culture and Conditions for Transductions and Infections

Polarized primary HAE cells were generated from lung donors and transplant airway tissue as described, e.g., in Karp et al. Methods Mol. Biol. 188:115-137, 2002. The HAE cells were grown on 6.5-mm diameter transwells with 0.4 μm diameter pores (Corning) in media containing USG. All cultures were estimated to contain approximately ˜7.5e5 cells at the time of reporter AAV transduction.

Non-CF HAE (donor code: B-7-19, B-13-19, B-15-19, B-16-19), and CF HAE (donor code: CB-32-18, CBF-4-19) with the genotype F508del/F508del were used in this study.

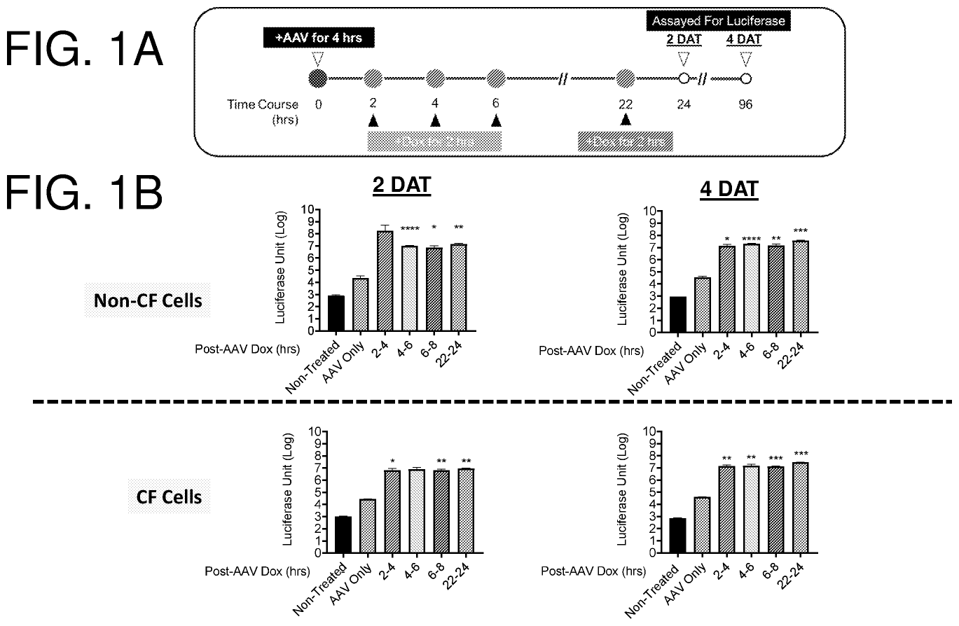

Cells were transduced with the reporter AV.TL65-CBA-mCherry-SP183 at a Multiplicity of Infection (MOI; ratio of viral vector genomes (vg) to cells) of approximately 6,600 and incubated for 4 or 16 hours, at which time the excess liquid was removed from the apical surface. Reporter rAAV was diluted in USG medium. Doxorubicin, at a concentration of 5 μM (equivalent to 2.9 μg/ml), was applied for 2 hours at different times post-AAV exposure, as summarized in Table 1 and represented in the schematics shown in FIGS. 1-3. For the “no AAV” (i.e., no treatment) and “AAV only” control conditions, 2 transwells (N=2) were used per condition. In other treatments (i.e., “AAV plus doxorubicin”), at least 4 transwells (N=4) per condition were analyzed.

TABLE 1
Summary of experimental paradigms: Duration of AAV incubation, followed by
timepoint for onset of 2-hour doxorubicin treatment, post-AAV addition.

| | | | Time-point post- | | |
|---|---|---|---|---|---|
| | | Length of | AAV exposure of | | |
| | | exposure to | the 2-hour | | Luciferase |
| | | AV.TL65-CBA- | doxorubicin | Cell | activity |
| FIG. | Study paradigm | mCherry-SP183 | treatment | type | determined at |
| 1 | Short time course | 4 hours | 2, 4, 6, and 22 | Non-CF | 2 and 4 Days |
| | of doxorubicin | | hours post- | and CF | After |
| | treatment | | exposure | | Transduction |
| | | | | | (DAT) |
| 2 | Intermediate time | 16 hours | 14, 16, 18, and 22 | Non-CF | 2 DAT |
| | course of | | hours post- | | |
| | doxorubicin | | exposure | | |
| | treatment | | | | |
| 3 | Extended time | 16 hours | 16, 40, and 88 | Non-CF | 4, 6 and 8 |
| | course of | | hours post- | | DAT |
| | doxorubicin | | exposure | | |
| | treatment | | | | |

In all cases, the reporter AV.TL65-CBA-mCherry-SP183 was added apically to HAE cells in a volume of 50 μL and incubated for either 4 or 16 hours and then removed by aspiration. Doxorubicin (5 μM) was then added to the basal chamber at various time-points post-AAV exposure (see summary in Table 1 and schematics in FIGS. 1-3). In all cases, doxorubicin treatment lasted for 2 hours. At the specified time-points, all solutions in the bottom chambers were removed and fresh USG medium without doxorubicin was added to the bottom chamber of each transwell. At the indicated time-points (i.e., 2, 4, 6, or 8 Days After Transduction, DAT), a sample of 200 μL was collected from the basal chamber of each transwell and used to determine luciferase activity as described below. Complete media change was also performed at each of these time points.

Measurement of Luciferase Activity

At days 2, 4, 6 and/or 8 Days After Transduction (DAT), luciferase activity was measured by taking a 50 μl aliquot from the 200 μL sample of conditioned media collected from the basal compartment of the transwells. This 50 μl aliquot was mixed with 10 μL of substrate/buffer mix (kit catalog number E2810, Promega Corporation, Madison, WI), as indicated by the manufacturer (Promega Luciferase Assay System Technical Manual). Luminescence was measured in a luminometer Promega GloMax™ 20/20, following manufacturer's instructions.

Statistical Analysis

Statistical analyses were performed with GraphPad Prism 8.4.3 (GraphPad Software, Inc., San Diego, CA). Luciferase activity was compared between treatment groups (AAV exposure, AAV plus doxorubicin) and control untreated cells (no AAV, no doxorubicin) using a two-tailed, unpaired T-test. A second analysis compared the AAV+doxorubicin to AAV alone groups. Differences between groups were considered to be significant at a p value of <0.05.

Results

To identify the effect of time of addition of doxorubicin post-AAV treatment of HAE cells, investigations were conducted using AV.TL65-CBA-mCherry-SP183, a reporter AAV construct with the same capsid as SP-101 but carrying Gaussia luciferase as a reporter under the control of the same promoter that drives hCFTRΔR expression in SP-101. A working concentration of the augmenter doxorubicin (5 μM) was identified based on non-toxic responses in HAE cells (namely >200-fold increase in transduction fold and <50% increase over baseline LDH release).

The combination of AAV transduction and augmenter treatment was explored in three experimental paradigms in non-CF and/or CF cells to establish an optimized window of treatment with doxorubicin, as summarized in the paragraph below and in Table 1 and further illustrated by the schematics shown in FIGS. 1A, 2A, and 3A. Altogether, these experiments explored the effect of a 2-hour treatment with doxorubicin between 2 and 88 hours post-AAV exposure.

In FIGS. 1A and 1B, a short time course study was based on exposure of HAE cultures with AV.TL65-CBA-mCherry-SP183 for 4 hours (MOI˜6,600 vg/cell, apical side), with 2-hour treatments of doxorubicin (5 μM, added to media on basal side) at timepoints of 2, 4, 6, and 22 hours post-AAV addition. This experiment was conducted both in normal (non-CF) and in CF HAE. Luciferase activity was determined at 2 and 4 Days After Transduction (DAT). For both the non-CF and CF HAE cultures, exposure to doxorubicin at each specified timepoint generally resulted in a significantly higher luciferase signal than the no-AAV control cultures (FIG. 1B) and AAV-only conditions for each HAE cell type. Altogether, the results show that the time-point of the 2-hour treatment with doxorubicin within this time frame did not significantly change the production of luciferase signal under these conditions either in normal non-CF or CF cells.

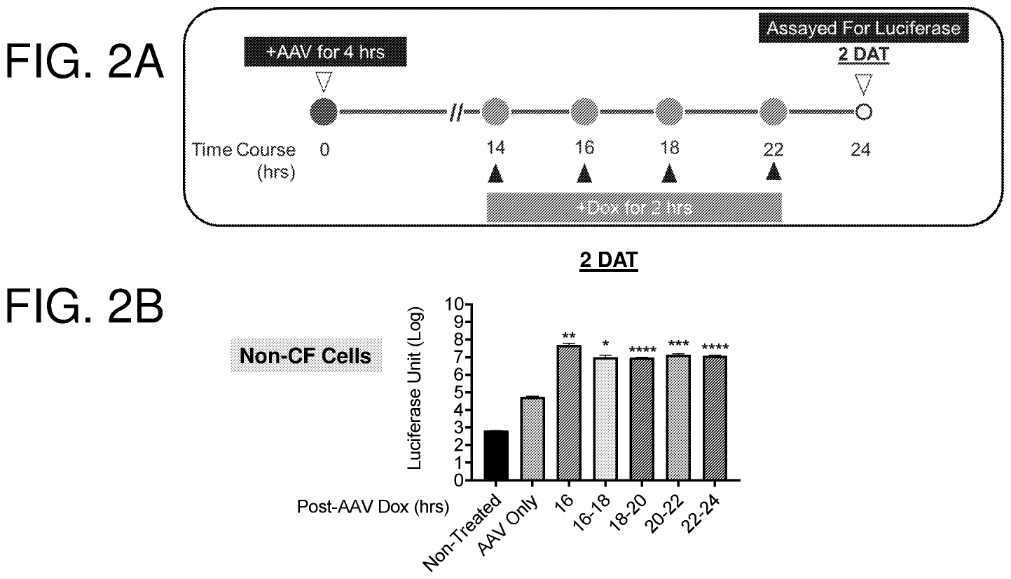

In FIGS. 2A and 2B, an intermediate time course study was based on exposure of HAE cultures with AV.TL65-CBA-mCherry-SP183 for 16 hours (MOI˜6,600 vg/cell, apical side), followed by a 2-hour treatment with doxorubicin (5 μM, added to media on basal side) at time-points of 14, 16, 18, and 22 hours post-AAV addition. This experiment was conducted in normal (non-CF) HAE and luciferase activity was determined at 2 DAT. Similar to the short time course experiment, treatment with doxorubicin generally resulted in significantly higher luciferase signal in all cultures relative to the no-AAV control (FIG. 2B) and AAV-only conditions. Additionally, the results show that the time-point of the 2-hour treatment with doxorubicin within the time frame of 14 to 22 hours (post-AAV addition) did not significantly affect the production of luciferase signal in this experiment. Furthermore, the 22 h time-point of treatment with doxorubicin resulted in a luciferase signal consistent with the results observed in the previous experiment for that particular time-point (see FIGS. 1A and 1B).

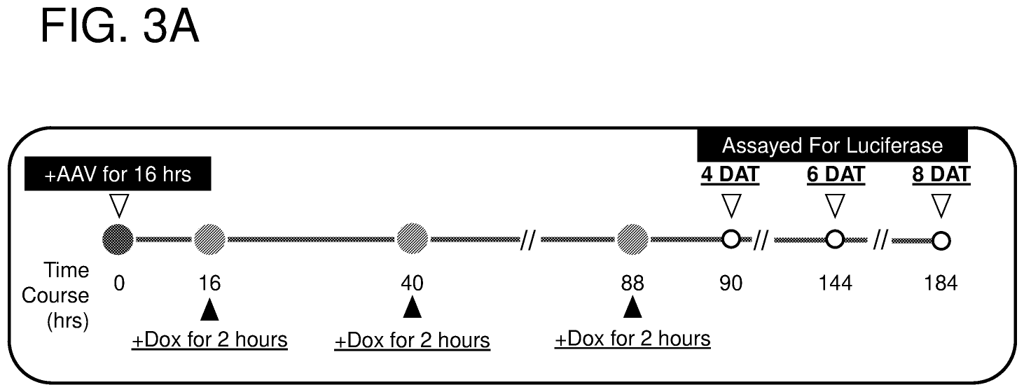

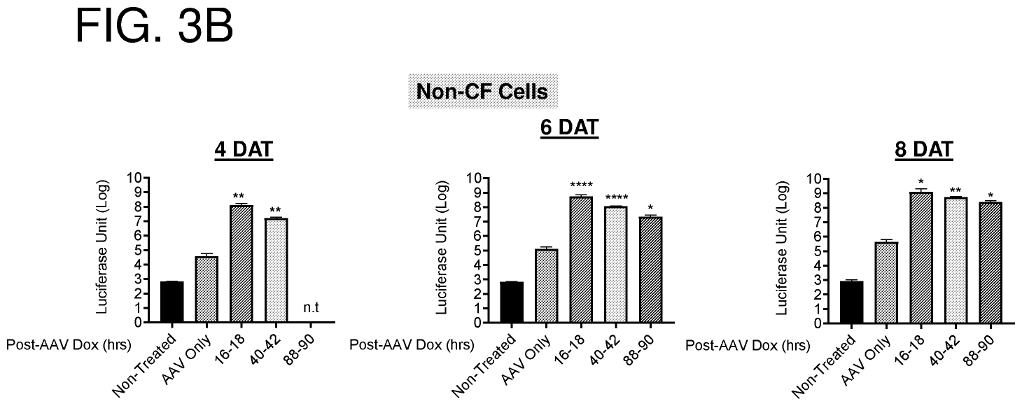

In FIGS. 3A and 3B, an extended time course study was based on exposure of HAE cultures with AV.TL65-CBA-mCherry-SP183 for 16 hours (MOI˜6,600 vg/cell, apical side), followed by a 2-hour treatment with doxorubicin (5 μM, added to media on basal side) at time-points of 16, 40, and 88 hours post-AAV addition. This experiment was conducted in normal (non-CF) HAE, and luciferase activity was determined at 4, 6, and 8 DAT. The results in this data set indicate that treatment with doxorubicin generally resulted in a significantly higher luciferase signal when compared to control non-AAV (FIG. 3B) and AAV-only controls.

Addition of doxorubicin to the basal side media for 2 hours immediately following the 16 hour incubation with AAV, resulted in luciferase expression on DAT 4 comparable to that observed with the previous 4 hours AAV exposure on DAT 2 (intermediate time course, FIGS. 2A and 2B) Furthermore, the level of luciferase expression observed in the '16 hours AAV exposure followed by 2 hours exposure to doxorubicin′ on DAT 4 is comparable to the expression levels observed in each of the AAV+doxorubicin cultures evaluated in the short time course experiment, on both DAT 2 and DAT 4 (FIGS. 1A and 1B). Increasing the time separation between exposure to AAV and administration of doxorubicin to either 40 or 88 hours still resulted in significant levels of luciferase expression relative to the AAV-only culture, although an overall trend to slightly reduced levels of luciferase expression was observed as the time of separation increased. Finally, the overall pattern of luciferase signal relative to the timepoints of observation (DAT 4, 6 and 8) remained consistent in this extended time course experiment, with the levels of luciferase expression remaining constant for each given culture condition over the duration of the 4-day measurement window.

In summary, in the experiments described in this Example and shown in FIGS. 1-3, transduction efficiency with the reporter AAV was generally significantly increased by treatment of airway epithelial cells with the augmenter doxorubicin.

### CONCLUSIONS

Exposure of both normal (non-CF) and CF HAE to the recombinant reporter AV.TL65-CBA-mCherry-SP183, at an MOI of 6,600, results in demonstrable luciferase signal that can be measured in the basal conditioned media from these differentiated and polarized airway epithelial cell cultures. Furthermore, the data show that transduction efficiency was generally significantly increased by treatment of these HAE cultures with a 2-hour exposure to the augmenter doxorubicin (5 μM), compared to control untreated cells and AAV-only treated cells, in all experiments.

Furthermore, under the experimental conditions employed in these investigations, the augmenter doxorubicin can be administered to HAE cells within a window of at least 40 hours post-AAV treatment without significantly altering the levels of transduction observed in comparison to earlier time points.

The reporter AV.TL65-CBA-mCherry-SP183 is an excellent surrogate for SP-101 (AAV2.5T-SP183-hCFTRΔR) based on shared characteristics, namely the capsid AV.TL65 and the promoter F5tg83/SP183 which drives the expression of the transgene Gaussia luciferase and human CFTRΔR respectively. Based on these attributes, the data presented here indicate that doxorubicin can be administered post-AAV exposure at various times within at least a 40-hour window without significant loss of activity. Taken altogether, the data presented in this Example establish a time window for enhanced effects of transduction by treatment with the small molecule augmenter doxorubicin.

### Example 3: Additional Data for Studies Described in Example 1

In this Example, additional data and results relating to the studies described in Example 1 are provided.

CF HAE Study

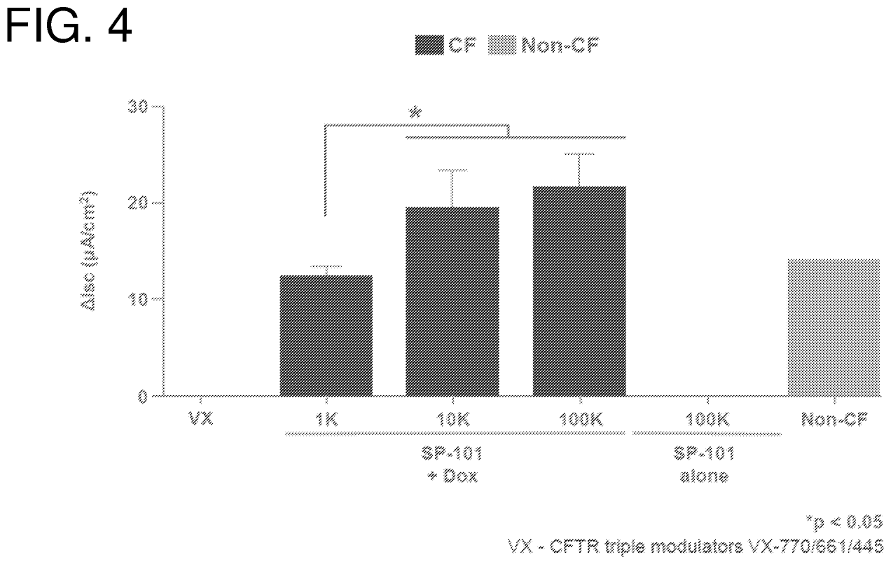

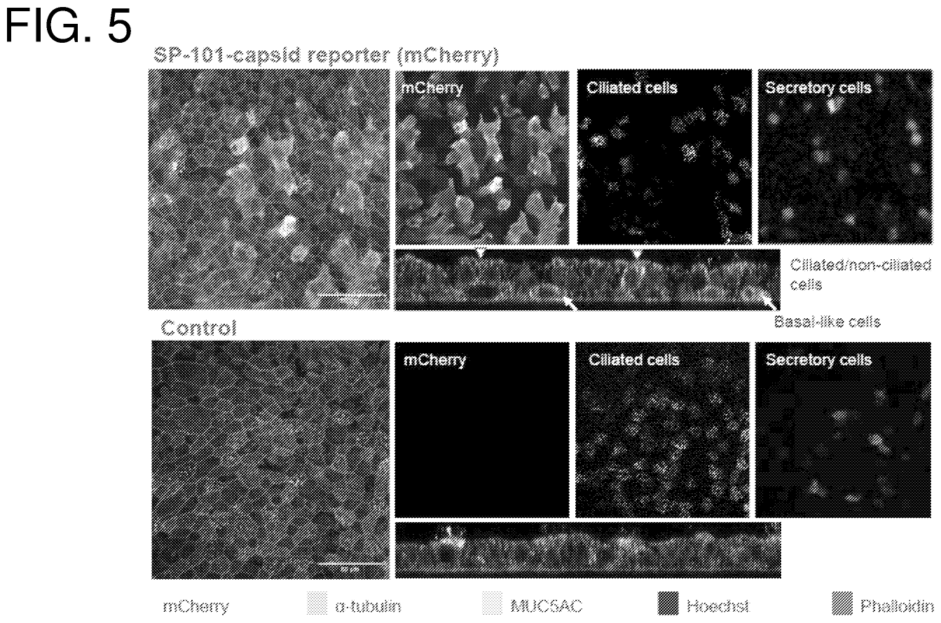

As is described in Example 1, SP-101 (AV.TL65-SP183-hCFTRΔR) was tropic to and corrected CF HAE. FIG. 4 shows that apical SP-101 demonstrated a dose-dependent functional correction of primary CF HAE. In contrast to CFTR modulators VX-770/661/445 that did not restore function in donors with class I mutations (W1282X/R1162X), treatment with SP-101 (MOI 1 K, 10K, 100K)+doxorubicin (5 μM) significantly increased currents in a dose-dependent manner to levels similar to non-CF HAE. FIG. 5 shows that SP-101-capsid reporter encoding mCherry transduced many epithelial cell types in CF HAE (F508del/F508del). SP-101-reporter (mCherry, yellow) showed >30% positive cells that colocalized with markers for ciliated (α-tubulin, white) or secretory cells (MUCSAC, teal) or did not colocalize with any cell type markers (non-ciliated or basally-oriented cells).

Ferret Study

As is also described in Example 1, ferrets can be used as a model to evaluate inhaled SP-101. AV.TL65 is tropic to ferret airway cells, and the CF ferret model recapitulates human CF lung pathology.

Additionally, SP-101 can be administered to ferrets via inhalation.

Additional Materials and Methods for Ferret Study Non-CF and CF ferrets were exposed to nebulized SP-101 or placebo, followed by doxorubicin or placebo on Day 1. Animals were necropsied 2 or 12 weeks post-exposure and tissues harvested for in situ hybridization (ISH) or determination of hCFTRΔR mRNA copy count. The CF ferrets have the G551 D allele, which corresponds to a class III mutation.

ISH: Sections from formalin-fixed, paraffin-embedded lung were evaluated by RNAScope™ ISH assay, using zz-probes designed to the sense strand unique regions of the SP-101 vector genome.

hCFTRΔR mRNA copies: RNA was isolated, using a DNase procedure to ensure removal of vector genomes, from 25-50 mg samples taken from 9 different regions of the airway (3 from tracheal, 4 from bronchial, 2 from alveolar/lobe regions). qPCR+/− reverse transcriptase was performed with primers and a probe for a unique region of the hCFTRΔR mRNA. No signal was observed in the absence of reverse transcriptase, indicating the complete removal of vector genomes. Data are shown as box and whisker plots around the median value (hCFTRΔR copy count normalized to 500 ng total RNA).

Additional Results for Ferret Study

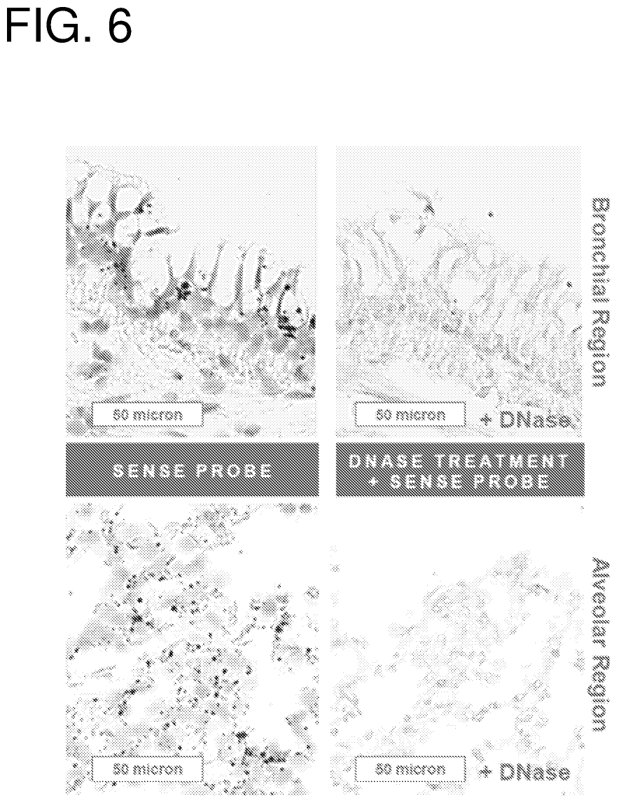

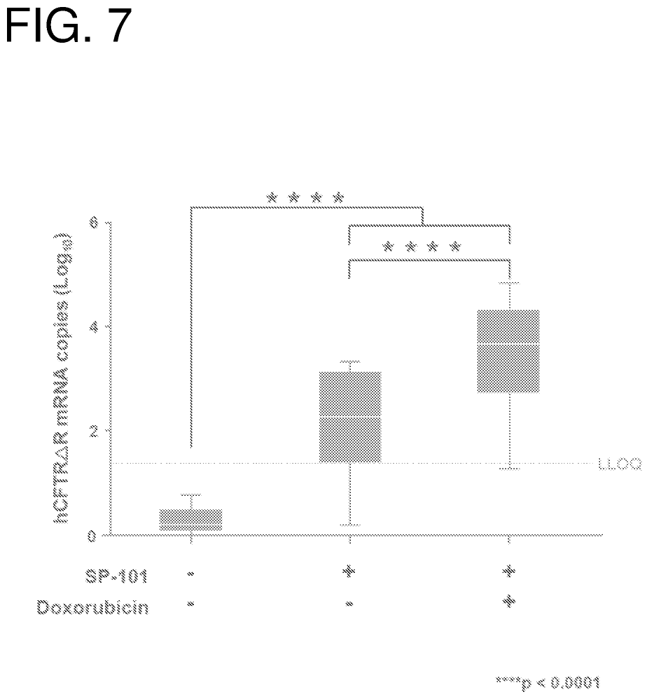

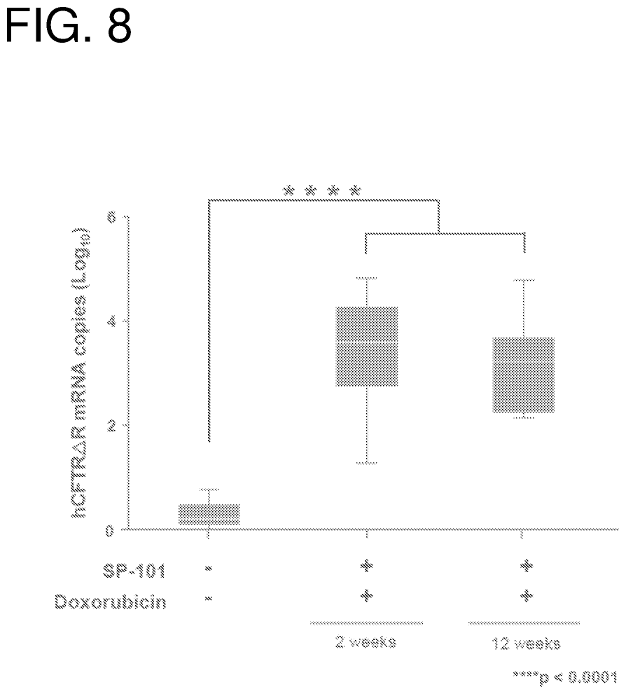

As is described in Example 1, hCFTRΔR mRNA expression was enhanced by doxorubicin and was durable. FIG. 6 shows that SP-101 vector genomes were abundant in many regions of non-CF ferret lungs. SP-101 vector genomes (red dots) were detected in multiple cells whereas pretreatment with DNase did not show staining indicating the specificity of staining. Additionally, FIG. 7 shows that hCFTRΔR mRNA expression was increased >10 fold by administration of doxorubicin and was durable in non-CF ferret lungs (FIG. 8). In contrast to control samples, hCFTRΔR mRNA was detected in the majority of samples from animals exposed to SP-101 alone. However, hCFTRΔR mRNA was >10 fold higher in samples from animals exposed to the same amount of SP-101 followed by doxorubicin (p<0.0001). Moreover, hCFTRΔR mRNA did not significantly decrease 12 weeks (end of study) post-administration, indicating durable expression (FIG. 8). Additionally, hCFTRΔR mRNA expression increased with increasing doses of SP-101/doxorubicin and was highest in lung tissues.

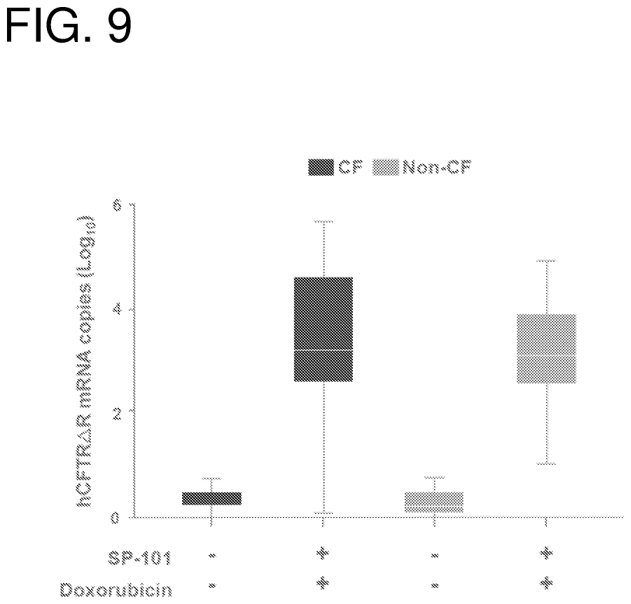

Additionally, as is also described in Example 1, the CF ferret lung was permissive for SP-101. hCFTRΔR mRNA expression was similar in the lungs of CF and non-CF ferrets (FIG. 9). In contrast to control animals (diluent only), hCFTRΔR mRNA was detectable to a similar extent in both CF (G551 D) and non-CF animals, indicating that the CF lung is not an additional barrier to SP-101.

### SUMMARY

As is described in Example 1, SP-101 functionally corrected CF HAE. SP-101 was tropic to many human epithelial types. hCFTRΔR expression and CF correction were dose responsive and durable. Additionally, hCFTRΔR mRNA expression was similar in CF and non-CF ferrets, indicating that the CF airway is not an additional barrier to SP-101.

### Example 4: hCFTRΔR Expression and Correction of Human CF Airway Epithelia Correlate with SP-101 Multiplicity of Infection and Doxorubicin Dose

Summary

SP-101 (AAV2.5T-SP183-hCFTRΔR) can be used as an inhalation treatment for people with cystic fibrosis (CF) in a mutation agnostic manner. We have found that co-administration of the small molecule enhancer doxorubicin is important for robust transgene expression. As is described above, upon apical inoculation of CF HAE, about 30-40% of the cells, including ciliated, secretory and basal cells, were transduced using a AAV2.5T-mCherry reporter in the presence of doxorubicin. Dose-response relationships for SP-101 and doxorubicin were further investigated in apically transduced CF HAE at day 7 post transduction. Forskolin-induced CFTR chloride conductance, measured via Ussing chamber assay, increased with increasing SP-101 multiplicity of infection (MOI) in the presence of doxorubicin and correlated with increasing vector copy numbers (VCN, measured by digital droplet PCR (ddPCR)) and hCFTRΔR mRNA expression (measured by RT-qPCR). Doxorubicin also demonstrated a dose-dependent increase of SP-101-mediated hCFTRΔR mRNA expression and chloride conductance. Even low concentrations of doxorubicin treatment were able to partially restore CFTR-mediated chloride conductance with low SP-101 MOI. However, despite a significant impact on hCFTRΔR mRNA and chloride conductance, doxorubicin dose did not significantly alter VCN. These data also indicate that dosage regimens with higher doses of SP-101 and lower doses of doxorubicin may be equivalent to dosage regimens with lower doses of SP-101 and higher doses of doxorubicin.

Materials and Methods

This Example describes data from 2 human donors with class I CFTR mutations (W1282X/R1162X). Cells were allowed to polarize and differentiate into epithelia. Epithelia were transduced from the apical side with SP-101+/− doxorubicin. The amount of SP-101 and/or doxorubicin was varied.

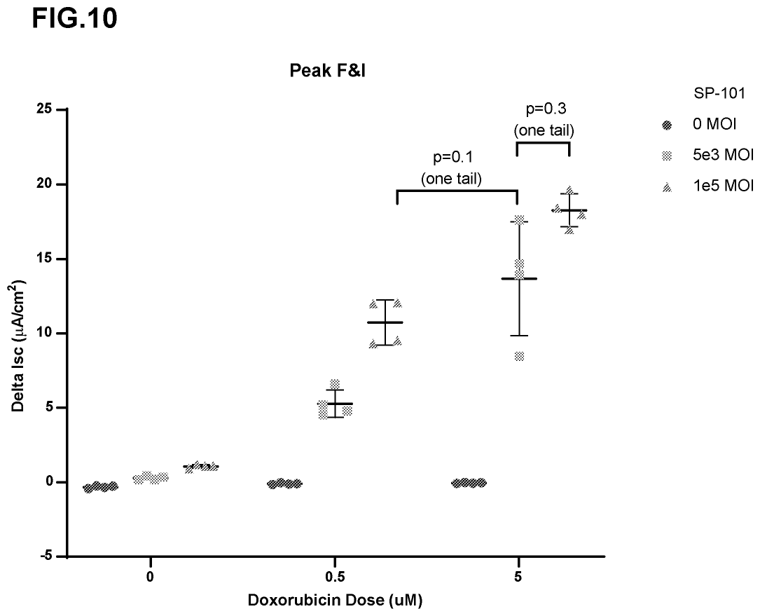

Donor 1 (FIGS. 10-12) demonstrates the impact of doxorubicin treatment level (0 μM, 0.5 μM, and 5 μM) on SP-101 vector copy number, mRNA levels, and functional correction by Ussing chamber assay in CF HAE with class I mutations. SP-101 was tested at MOI 0, 5e3, and 1e5 vector genomes/cell. SP-101 was added to the apical side, and doxorubicin was added to the basal side, all dissolved in UNC air liquid interface (ALI) media (see, e.g., Fulcher et al. Methods MoL. Biol. 945:109-121, 2013). SP-101 and doxorubicin were added simultaneously and incubated for 16 h before wash-out. The primary cells were KKD030O (W1282X/R1162X).

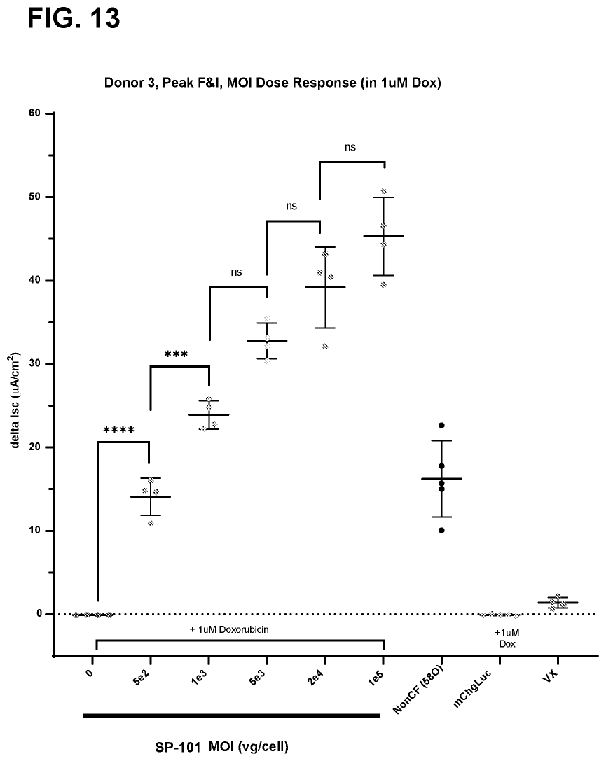

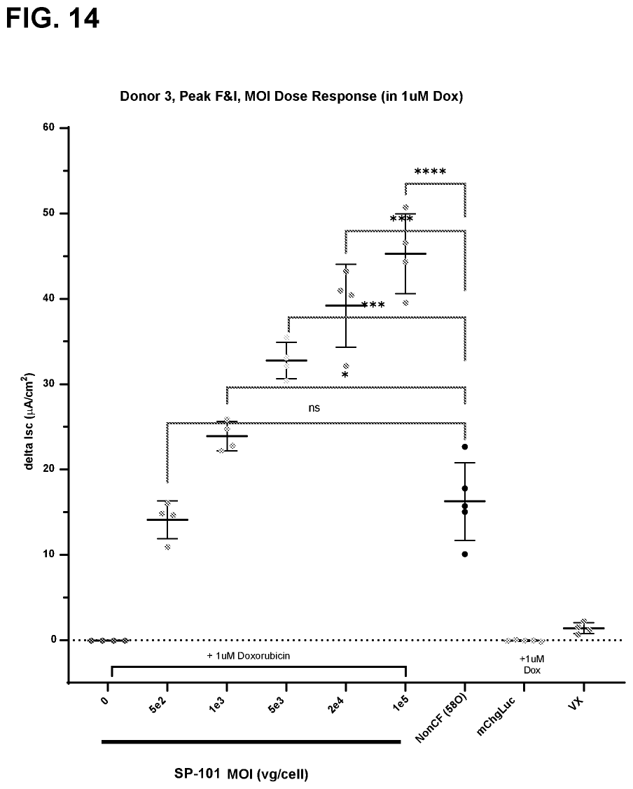

Donor 2 (FIGS. 13 and 14) demonstrates functional correction/response based on SP-101 MOI dose response at 1 μM doxorubicin. SP-101 was added to the apical side, and doxorubicin was added to the basal side, all dissolved in UNC ALI media. SP-101 and doxorubicin were simultaneously and incubated for 16 h before wash-out. The primary cells were KKD029O (W1282X/R1162X).

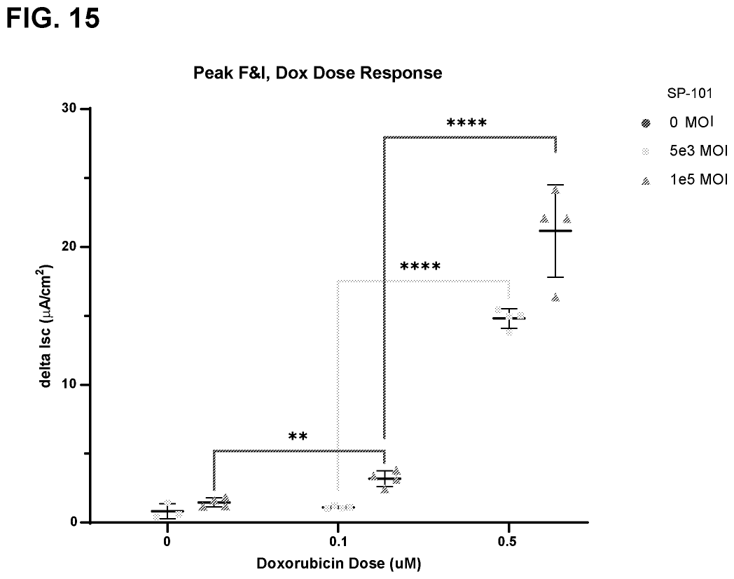

Donor 2 (FIG. 15) demonstrates the impact of lower doxorubicin treatment levels on SP-101 MOI and function correction.

Results

FIG. 10 shows that a low level of doxorubicin was sufficient to enhance the ability for SP-101 to demonstrate functional activity in class I CF HAE by Ussing chamber analysis. Moreover, a doxorubicin treatment level response was observed for low and high SP-101 MOI. These data indicate that Ussing currents increase with increasing SP-101 MOI and doxorubicin concentration. There was no significant difference between higher MOI (1e5) with lower doxorubicin (0.5 μM) and lower MOI (5e3) and higher doxorubicin (5 μM), indicating that both MOI and doxorubicin cooperate with each other to yield a similar functional outcome in vitro.

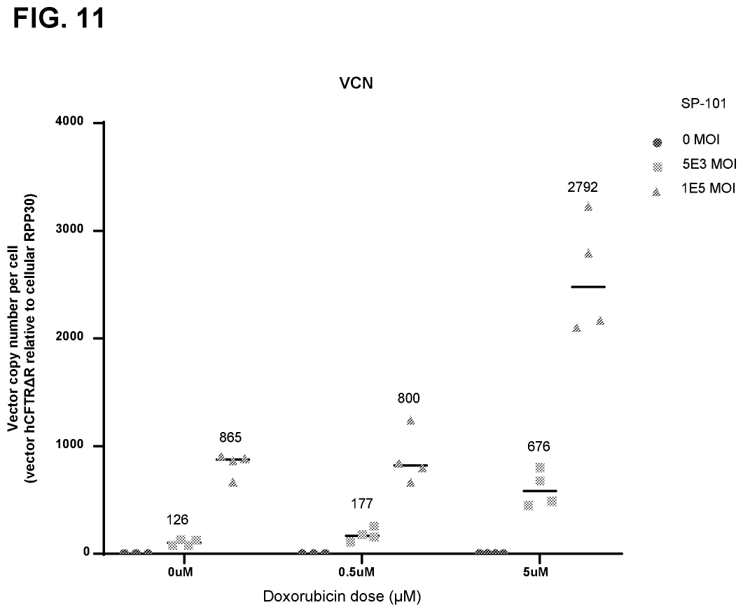

FIG. 11 shows that VCN correlated with the SP-101 MOI, but not with doxorubicin concentrations lower than 5 μM.

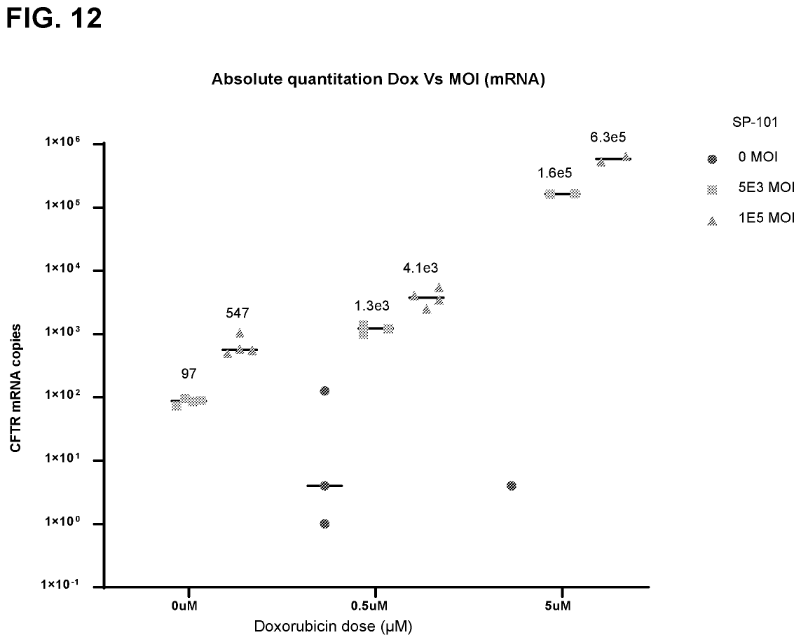

FIG. 12 shows that hCFTRΔR mRNA correlated positively with SP-101 MOI and doxorubicin treatment level.

FIG. 13 shows that a SP-101 MOI dose response was observed in this donor at 1 μM doxorubicin. A low SP-101 MOI (5e2 MOI)+1 μM doxorubicin demonstrated similar CFTR activity as non-CF HAE. The Vertex modulator treatment did not improve CFTR current. These data indicate that Ussing currents increased with increasing SP-101 MOI at 1 μM doxorubicin concentration.

FIG. 14 shows that all doses of AAV above 5e2 MOI stimulated Ussing current significantly greater than non-CF HAE. FIG. 14 illustrates the same data from FIG. 13 using a different statistical approach. In particular, a T-test was used to compare non-CF to SP-101+1 μM doxorubicin treated HAE. MOI 5e2 was similar to non-CF HAE while all other MOIs had significantly higher CFTR-related short circuit current.

FIG. 15 shows that a doxorubicin dose response and a SP-101 MOI dose response were observed in this donor. At 1e5 MOI, low level of doxorubicin (0.1 μM) was sufficient to result in increased CFTR activity by Ussing relative to no (0 μM) doxorubicin. MOI 5e3+0.5 μM doxorubicin was similar to non-CF HAE as shown in FIGS. 13 and 14. These data indicate that at each SP-101 treatment level, Ussing currents increased with increasing doxorubicin concentration. In comparison to the previous figures for this donor (FIGS. 13 and 14), MOI 5e2+1 μM Dox was similar to MOI 5e3+0.5 μM Dox, which was similar to non-CF HAE, indicating that SP-101 MOI and doxorubicin treatment level may be able cooperate with each other for the same or equivalent CFTR functional outcomes in vitro.

### Example 5: Additional Results for Studies Described in Example 4

In this Example, additional data and results relating to the studies described in Example 4 are provided. In particular, these results demonstrate that co-administration of SP-101 with doxorubicin restored forskolin-induced CFTR-mediated chloride conductance in CF-HAE cultures with class I, class II, and class III mutations.

Additional Methods

SP-101 was applied to the apical surfaces of CF-HAE cultured at the air-liquid interface with or without doxorubicin added to the basal media. Forskolin-induced CFTR chloride conductance, measured via Ussing chamber assay, was compared to cellular vector copy number (VCN), measured via ddPCR, and mRNA expression, measured by RT-qPCR, at day 7 post transduction. Cellular integrity was measured by evaluating transepithelial electrical resistance (TEER) and lactate dehydrogenase (LDH) levels in culture media. Tropism to CF-HAE was determined by five-color wholemount immunostaining and confocal microscopy for mCherry positive cells and various cell-type markers after transduction with an AAV2.5T-mCherry reporter vector.

Additional Results

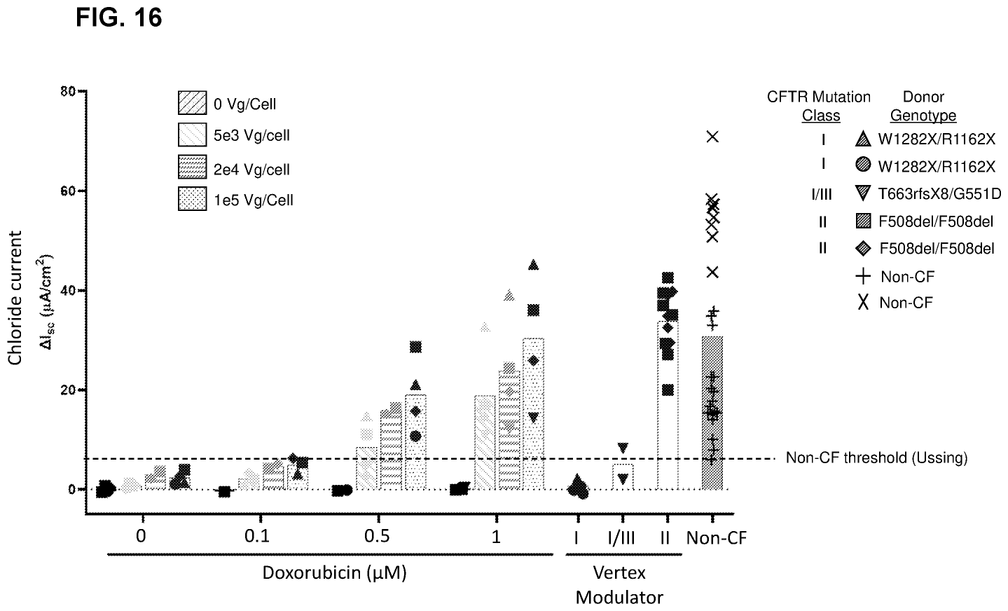

In the presence of doxorubicin, SP-101 restored forskolin-induced CFTR-mediated chloride conductance in all mutation classes tested to levels comparable to non-CF controls or small molecule modulator controls, with a Class I mutation donor showing the largest chloride response at the highest multiplicity of infection (MOI; 1e5 vg/cell). Functional chloride correction increased with increasing MOI, correlating with increasing VCN and hCFTRΔR mRNA expression. Similarly, increasing doxorubicin concentrations increased chloride conductance and hCFTRΔR mRNA expression in CF-HAE without significantly affecting VCN. Low concentrations of doxorubicin (as low as 0.5 μM) were able to restore CFTR-mediated chloride conductance with as little MOI as 5e3 vg/cell. TEER and LDH levels were not significantly different from control epithelia indicating no obvious toxicity as a result of treatment. Using an AAV2.5T-mCherry reporter vector, we showed that approximately 30-40% of CF-HAE cells, including ciliated, secretory, and basal-like cells, expressed the reporter gene under the same experimental conditions, providing insight into which and how many cells contribute to the correction of CF observed in vitro. As shown in FIG. 16, increasing doxorubicin and SP-101 vector doses increased the correction of CF human airway epithelia, derived from donors with class I, II or III mutations, to levels similar to non-CF epithelia. In contrast, Vertex modulator (VX-770/661/445) treatment did not correct epithelia with two class I mutations, and only partially corrected epithelia heterozygous for class I and III mutations. The only epithelia that the Vertex modulator treatment could fully correct were epithelia with two class II mutations. Therefore, the present approach allows for improved correction of CF human airway epithelia compared to existing approaches, particularly for patients whose genotypes include class I mutations and/or class III mutations.

### Example 6: Additional Results from Ferret Studies Described in Example 1 and 3

In this Example, additional data and results relating to the ferret studies described in Examples 1 and 3 are provided.

Similar dose-response relationships as described in Examples 4 and 5 were observed in the airways of wild-type ferrets. Inhaled administration of increasing doses of SP-101 and doxorubicin resulted in dose-dependent increases of hCFTRΔR mRNA expression. The highest levels were observed in the lungs and bronchi, followed by trachea and nose. Expression levels of hCFTRΔR mRNA were comparable to those of endogenous ferret CFTR at 14 days post-administration. hCFTRΔR mRNA expression started as early as 48 h (earliest time point investigated) and persisted up to 3 months (longest time-point investigated). Comparable hCFTRΔR mRNA expression was also evident in the airways of CF ferrets, indicating successful transduction despite pre-existing mucus accumulation.

SEQUENCE LISTING

| SEQ | | |
|---|---|---|
| ID NO | Name | Sequence |
| 1 | F5 | GAATTCGTGGTGAGCGTCTGGGCATGTCTGGGCATGTCTGGGCATGTCTG |
| | Enhancer | GGCATGTCGGGCATTCTGGGCGTCTGGGCATGTCTGGGCATGTCTGGGC |
| | with 5′ | ATCTCGAG |
| | EcoRI and | |
| | 3′ Xhol | |
| | sites | |
| 2 | tg83 | AACGGTGACGTGCACGCGTGGGCGGAGCCATCACGCAGGTTGCTATATA |
| | Promoter | AGCAGAGCTCGTTTAGTGAACCGTCAGA |
| 3 | 5′-UTR | GTCGAGCCCGAGAGACC |
| 4 | hCFTRΔR | ATGCAGAGGTCGCCTCTGGAAAAGGCCAGCGTTGTCTCCAAACTTTTTTTC |
| | | AGCTGGACCAGACCAATTTTGAGGAAAGGATACAGACAGCGCCTGGAATT |
| | | GTCAGACATATACCAAATCCCTTCTGTTGATTCTGCTGACAATCTATCTGAA |
| | | AAATTGGAAAGAGAATGGGATAGAGAGCTGGCTTCAAAGAAAAATCCTAAA |
| | | CTCATTAATGCCCTTCGGCGATGTTTTTTCTGGAGATTTATGTTCTATGGAA |
| | | TCTTTTTATATTTAGGGGAAGTCACCAAAGCAGTACAGCCTCTCTTACTGG |
| | | GAAGAATCATAGCTTCCTATGACCCGGATAACAAGGAGGAACGCTCTATC |
| | | GCGATTTATCTAGGCATAGGCTTATGCCTTCTCTTTATTGTGAGGACACTG |
| | | CTCCTACACCCAGCCATTTTTGGCCTTCATCACATTGGAATGCAGATGAGA |
| | | ATAGCTATGTTTAGTTTGATTTATAAGAAGACTTTAAAGCTGTCAAGCCGTG |
| | | TTCTAGATAAAATAAGTATTGGACAACTTGTTAGTCTCCTTTCCAACAACCT |
| | | GAACAAATTTGATGAAGGACTTGCATTGGCACATTTCGTGTGGATCGCTCC |
| | | TTTGCAAGTGGCACTCCTCATGGGGCTAATCTGGGAGTTGTTACAGGCGT |
| | | CTGCCTTCTGTGGACTTGGTTTCCTGATAGTCCTTGCCCTTTTTCAGGCTG |
| | | GGCTAGGGAGAATGATGATGAAGTACAGAGATCAGAGAGCTGGGAAGATC |
| | | AGTGAAAGACTTGTGATTACCTCAGAAATGATCGAGAACATCCAATCTGTT |
| | | AAGGCATACTGCTGGGAAGAAGCAATGGAAAAAATGATTGAAAACTTAAGA |
| | | CAAACAGAACTGAAACTGACTCGGAAGGCAGCCTATGTGAGATACTTCAAT |
| | | AGCTCAGCCTTCTTCTTCTCAGGGTTCTTTGTGGTGTTTTTATCTGTGCTTC |
| | | CCTATGCACTAATCAAAGGAATCATCCTCCGGAAAATATTCACCACCATCT |
| | | CATTCTGCATTGTTCTGCGCATGGCGGTCACTCGGCAATTTCCCTGGGCT |
| | | GTACAAACATGGTATGACTCTCTTGGAGCAATAAACAAAATACAGGATTTC |
| | | TTACAAAAGCAAGAATATAAGACATTGGAATATAACTTAACGACTACAGAA |
| | | GTAGTGATGGAGAATGTAACAGCCTTCTGGGAGGAGGGATTTGGGGAATT |
| | | ATTTGAGAAAGCAAAACAAAACAATAACAATAGAAAAACTTCTAATGGTGAT |
| | | GACAGCCTCTTCTTCAGTAATTTCTCACTTCTTGGTACTCCTGTCCTGAAAG |
| | | ATATTAATTTCAAGATAGAAAGAGGACAGTTGTTGGCGGTTGCTGGATCCA |
| | | CTGGAGCAGGCAAGACTTCACTTCTAATGATGATTATGGGAGAACTGGAG |
| | | CCTTCAGAGGGTAAAATTAAGCACAGTGGAAGAATTTCATTCTGTTCTCAG |
| | | TTTTCCTGGATTATGCCTGGCACCATTAAAGAAAATATCATCTTTGGTGTTT |
| | | CCTATGATGAATATAGATACAGAAGCGTCATCAAAGCATGCCAACTAGAAG |
| | | AGGACATCTCCAAGTTTGCAGAGAAAGACAATATAGTTCTTGGAGAAGGTG |
| | | GAATCACACTGAGTGGAGGTCAACGAGCAAGAATTTCTTTAGCAAGAGCA |
| | | GTATACAAAGATGCTGATTTGTATTTATTAGACTCTCCTTTTGGATACCTAG |
| | | ATGTTTTAACAGAAAAAGAAATATTTGAAAGCTGTGTCTGTAAACTGATGGC |
| | | TAACAAAACTAGGATTTTGGTCACTTCTAAAATGGAACATTTAAAGAAAGCT |
| | | GACAAAATATTAATTTTGCATGAAGGTAGCAGCTATTTTTATGGGACATTTT |
| | | CAGAACTCCAAAATCTACAGCCAGACTTTAGCTCAAAACTCATGGGATGTG |
| | | ATTCTTTCGACCAATTTAGTGCAGAAAGAAGAAATTCAATCCTAACTGAGA |
| | | CCTTACACCGTTTCTCATTAGAAGGAGATGCTCCTGTCTCCTGGACAGAAA |
| | | CAAAAAAACAATCTTTTAAACAGACTGGAGAGTTTGGGGAAAAAAGGAAGA |
| | | ATTCTATTCTCAATCCAATCAACTCTACGCTTCAGGCACGAAGGAGGCAGT |
| | | CTGTCCTGAACCTGATGACACACTCAGTTAACCAAGGTCAGAACATTCACC |
| | | GAAAGACAACAGCATCCACACGAAAAGTGTCACTGGCCCCTCAGGCAAAC |
| | | TTGACTGAACTGGATATATATTCAAGAAGGTTATCTCAAGAAACTGGCTTG |
| | | GAAATAAGTGAAGAAATTAACGAAGAAGACTTAAAGGAGTGCCTTTTTGAT |
| | | GATATGGAGAGCATACCAGCAGTGACTACATGGAACACATACCTTCGATAT |
| | | ATTACTGTCCACAAGAGCTTAATTTTTGTGCTAATTTGGTGCTTAGTAATTT |
| | | TTCTGGCAGAGGTGGCTGCTTCTTTGGTTGTGCTGTGGCTCCTTGGAAAC |
| | | ACTCCTCTTCAAGACAAAGGGAATAGTACTCATAGTAGAAATAACAGCTAT |
| | | GCAGTGATTATCACCAGCACCAGTTCGTATTATGTGTTTTACATTTACGTG |
| | | GGAGTAGCCGACACTTTGCTTGCTATGGGATTCTTCAGAGGTCTACCACT |
| | | GGTGCATACTCTAATCACAGTGTCGAAAATTTTACACCACAAAATGTTACAT |
| | | TCTGTTCTTCAAGCACCTATGTCAACCCTCAACACGTTGAAAGCAGGTGGG |
| | | ATTCTTAATAGATTCTCCAAAGATATAGCAATTTTGGATGACCTTCTGCCTC |
| | | TTACCATATTTGACTTCATCCAGTTGTTATTAATTGTGATTGGAGCTATAGC |
| | | AGTTGTCGCAGTTTTACAACCCTACATCTTTGTTGCAACAGTGCCAGTGAT |
| | | AGTGGCTTTTATTATGTTGAGAGCATATTTCCTCCAAACCTCACAGCAACTC |
| | | AAACAACTGGAATCTGAAGGCAGGAGTCCAATTTTCACTCATCTTGTTACA |
| | | AGCTTAAAAGGACTATGGACACTTCGTGCCTTCGGACGGCAGCCTTACTTT |
| | | GAAACTCTGTTCCACAAAGCTCTGAATTTACATACTGCCAACTGGTTCTTGT |
| | | ACCTGTCAACACTGCGCTGGTTCCAAATGAGAATAGAAATGATTTTTGTCA |
| | | TCTTCTTCATTGCTGTTACCTTCATTTCCATTTTAACAACAGGAGAAGGAGA |
| | | AGGAAGAGTTGGTATTATCCTGACTTTAGCCATGAATATCATGAGTACATT |
| | | GCAGTGGGCTGTAAACTCCAGCATAGATGTGGATAGCTTGATGCGATCTG |
| | | TGAGCCGAGTCTTTAAGTTCATTGACATGCCAACAGAAGGTAAACCTACCA |
| | | AGTCAACCAAACCATACAAGAATGGCCAACTCTCGAAAGTTATGATTATTG |
| | | AGAATTCACACGTGAAGAAAGATGACATCTGGCCCTCAGGGGGCCAAATG |
| | | ACTGTCAAAGATCTCACAGCAAAATACACAGAAGGTGGAAATGCCATATTA |
| | | GAGAACATTTCCTTCTCAATAAGTCCTGGCCAGAGGGTGGGCCTCTTGGG |
| | | AAGAACTGGATCAGGGAAGAGTACTTTGTTATCAGCTTTTTTGAGACTACT |
| | | GAACACTGAAGGAGAAATCCAGATCGATGGTGTGTCTTGGGATTCAATAA |
| | | CTTTGCAACAGTGGAGGAAAGCCTTTGGAGTGATACCACAGAAAGTATTTA |
| | | TTTTTTCTGGAACATTTAGAAAAAACTTGGATCCCTATGAACAGTGGAGTGA |
| | | TCAAGAAATATGGAAAGTTGCAGATGAGGTTGGGCTCAGATCTGTGATAG |
| | | AACAGTTTCCTGGGAAGCTTGACTTTGTCCTTGTGGATGGGGGCTGTGTC |
| | | CTAAGCCATGGCCACAAGCAGTTGATGTGCTTGGCTAGATCTGTTCTCAGT |
| | | AAGGCGAAGATCTTGCTGCTTGATGAACCCAGTGCTCATTTGGATCCAGTA |
| | | ACATACCAAATAATTAGAAGAACTCTAAAACAAGCATTTGCTGATTGCACA |
| | | GTAATTCTCTGTGAACACAGGATAGAAGCAATGCTGGAATGCCAACAATTT |
| | | TTGGTCATAGAAGAGAACAAAGTGCGGCAGTACGATTCCATCCAGAAACT |
| | | GCTGAACGAGAGGAGCCTCTTCCGGCAAGCCATCAGCCCCTCCGACAGG |
| | | GTGAAGCTCTTTCCCCACCGGAACTCAAGCAAGTGCAAGTCTAAGCCCCA |
| | | GATTGCTGCTCTGAAAGAGGAGACAGAAGAAGAGGTGCAAGATACAAGGC |
| | | TTTAG |
| 5 | 3′-UTR | AGAGCAGCATAAATGTTGACATGGGACATTTGCTCATGGAATTGG |
| 6 | s-pA | AATAAAGAGCTCAGATGCATCGATCAGAGTGTGTTGGTTTTTTGTGTGTA |
| 7 | F5 | GAATTCGTGGTGAGCGTCTGGGCATGTCTGGGCATGTCTGGGCATGTCTG |
| | Enhancer, | GGCATGTCGGGCATTCTGGGCGTCTGGGCATGTCTGGGCATGTCTGGGC |
| | Tg83 | ATCTCGAGAACGGTGACGTGCACGCGTGGGCGGAGCCATCACGCAGGTT |
| | Promoter, | GCTATATAAGCAGAGCTCGTTTAGTGAACCGTCAGAGTCGAGCCCGAGAG |
| | 5′-UTR, | ACCATGCAGAGGTCGCCTCTGGAAAAGGCCAGCGTTGTCTCCAAACTTTT |
| | hCFTRΔR | TTTCAGCTGGACCAGACCAATTTTGAGGAAAGGATACAGACAGCGCCTGG |
| | | AATTGTCAGACATATACCAAATCCCTTCTGTTGATTCTGCTGACAATCTATC |
| | | TGAAAAATTGGAAAGAGAATGGGATAGAGAGCTGGCTTCAAAGAAAAATC |
| | | CTAAACTCATTAATGCCCTTCGGCGATGTTTTTTCTGGAGATTTATGTTCTA |
| | | TGGAATCTTTTTATATTTAGGGGAAGTCACCAAAGCAGTACAGCCTCTCTT |
| | | ACTGGGAAGAATCATAGCTTCCTATGACCCGGATAACAAGGAGGAACGCT |
| | | CTATCGCGATTTATCTAGGCATAGGCTTATGCCTTCTCTTTATTGTGAGGA |
| | | CACTGCTCCTACACCCAGCCATTTTTGGCCTTCATCACATTGGAATGCAGA |
| | | TGAGAATAGCTATGTTTAGTTTGATTTATAAGAAGACTTTAAAGCTGTCAAG |
| | | CCGTGTTCTAGATAAAATAAGTATTGGACAACTTGTTAGTCTCCTTTCCAAC |
| | | AACCTGAACAAATTTGATGAAGGACTTGCATTGGCACATTTCGTGTGGATC |
| | | GCTCCTTTGCAAGTGGCACTCCTCATGGGGCTAATCTGGGAGTTGTTACA |
| | | GGCGTCTGCCTTCTGTGGACTTGGTTTCCTGATAGTCCTTGCCCTTTTTCA |
| | | GGCTGGGCTAGGGAGAATGATGATGAAGTACAGAGATCAGAGAGCTGGG |
| | | AAGATCAGTGAAAGACTTGTGATTACCTCAGAAATGATCGAGAACATCCAA |
| | | TCTGTTAAGGCATACTGCTGGGAAGAAGCAATGGAAAAAATGATTGAAAAC |
| | | TTAAGACAAACAGAACTGAAACTGACTCGGAAGGCAGCCTATGTGAGATA |
| | | CTTCAATAGCTCAGCCTTCTTCTTCTCAGGGTTCTTTGTGGTGTTTTTATCT |
| | | GTGCTTCCCTATGCACTAATCAAAGGAATCATCCTCCGGAAAATATTCACC |
| | | ACCATCTCATTCTGCATTGTTCTGCGCATGGCGGTCACTCGGCAATTTCCC |
| | | TGGGCTGTACAAACATGGTATGACTCTCTTGGAGCAATAAACAAAATACAG |
| | | GATTTCTTACAAAAGCAAGAATATAAGACATTGGAATATAACTTAACGACTA |
| | | CAGAAGTAGTGATGGAGAATGTAACAGCCTTCTGGGAGGAGGGATTTGGG |
| | | GAATTATTTGAGAAAGCAAAACAAAACAATAACAATAGAAAAACTTCTAATG |
| | | GTGATGACAGCCTCTTCTTCAGTAATTTCTCACTTCTTGGTACTCCTGTCCT |
| | | GAAAGATATTAATTTCAAGATAGAAAGAGGACAGTTGTTGGCGGTTGCTGG |
| | | ATCCACTGGAGCAGGCAAGACTTCACTTCTAATGATGATTATGGGAGAACT |
| | | GGAGCCTTCAGAGGGTAAAATTAAGCACAGTGGAAGAATTTCATTCTGTTC |
| | | TCAGTTTTCCTGGATTATGCCTGGCACCATTAAAGAAAATATCATCTTTGGT |
| | | GTTTCCTATGATGAATATAGATACAGAAGCGTCATCAAAGCATGCCAACTA |
| | | GAAGAGGACATCTCCAAGTTTGCAGAGAAAGACAATATAGTTCTTGGAGAA |
| | | GGTGGAATCACACTGAGTGGAGGTCAACGAGCAAGAATTTCTTTAGCAAG |
| | | AGCAGTATACAAAGATGCTGATTTGTATTTATTAGACTCTCCTTTTGGATAC |
| | | CTAGATGTTTTAACAGAAAAAGAAATATTTGAAAGCTGTGTCTGTAAACTGA |
| | | TGGCTAACAAAACTAGGATTTTGGTCACTTCTAAAATGGAACATTTAAAGAA |
| | | AGCTGACAAAATATTAATTTTGCATGAAGGTAGCAGCTATTTTTATGGGACA |
| | | TTTTCAGAACTCCAAAATCTACAGCCAGACTTTAGCTCAAAACTCATGGGA |
| | | TGTGATTCTTTCGACCAATTTAGTGCAGAAAGAAGAAATTCAATCCTAACTG |
| | | AGACCTTACACCGTTTCTCATTAGAAGGAGATGCTCCTGTCTCCTGGACAG |
| | | AAACAAAAAAACAATCTTTTAAACAGACTGGAGAGTTTGGGGAAAAAAGGA |
| | | AGAATTCTATTCTCAATCCAATCAACTCTACGCTTCAGGCACGAAGGAGGC |
| | | AGTCTGTCCTGAACCTGATGACACACTCAGTTAACCAAGGTCAGAACATTC |
| | | ACCGAAAGACAACAGCATCCACACGAAAAGTGTCACTGGCCCCTCAGGCA |
| | | AACTTGACTGAACTGGATATATATTCAAGAAGGTTATCTCAAGAAACTGGC |
| | | TTGGAAATAAGTGAAGAAATTAACGAAGAAGACTTAAAGGAGTGCCTTTTT |
| | | GATGATATGGAGAGCATACCAGCAGTGACTACATGGAACACATACCTTCG |
| | | ATATATTACTGTCCACAAGAGCTTAATTTTTGTGCTAATTTGGTGCTTAGTA |
| | | ATTTTTCTGGCAGAGGTGGCTGCTTCTTTGGTTGTGCTGTGGCTCCTTGGA |
| | | AACACTCCTCTTCAAGACAAAGGGAATAGTACTCATAGTAGAAATAACAGC |
| | | TATGCAGTGATTATCACCAGCACCAGTTCGTATTATGTGTTTTACATTTACG |
| | | TGGGAGTAGCCGACACTTTGCTTGCTATGGGATTCTTCAGAGGTCTACCA |
| | | CTGGTGCATACTCTAATCACAGTGTCGAAAATTTTACACCACAAAATGTTAC |
| | | ATTCTGTTCTTCAAGCACCTATGTCAACCCTCAACACGTTGAAAGCAGGTG |
| | | GGATTCTTAATAGATTCTCCAAAGATATAGCAATTTTGGATGACCTTCTGCC |
| | | TCTTACCATATTTGACTTCATCCAGTTGTTATTAATTGTGATTGGAGCTATA |
| | | GCAGTTGTCGCAGTTTTACAACCCTACATCTTTGTTGCAACAGTGCCAGTG |
| | | ATAGTGGCTTTTATTATGTTGAGAGCATATTTCCTCCAAACCTCACAGCAAC |
| | | TCAAACAACTGGAATCTGAAGGCAGGAGTCCAATTTTCACTCATCTTGTTA |
| | | CAAGCTTAAAAGGACTATGGACACTTCGTGCCTTCGGACGGCAGCCTTAC |
| | | TTTGAAACTCTGTTCCACAAAGCTCTGAATTTACATACTGCCAACTGGTTCT |
| | | TGTACCTGTCAACACTGCGCTGGTTCCAAATGAGAATAGAAATGATTTTTG |
| | | TCATCTTCTTCATTGCTGTTACCTTCATTTCCATTTTAACAACAGGAGAAGG |
| | | AGAAGGAAGAGTTGGTATTATCCTGACTTTAGCCATGAATATCATGAGTAC |
| | | ATTGCAGTGGGCTGTAAACTCCAGCATAGATGTGGATAGCTTGATGCGAT |
| | | CTGTGAGCCGAGTCTTTAAGTTCATTGACATGCCAACAGAAGGTAAACCTA |
| | | CCAAGTCAACCAAACCATACAAGAATGGCCAACTCTCGAAAGTTATGATTA |
| | | TTGAGAATTCACACGTGAAGAAAGATGACATCTGGCCCTCAGGGGGCCAA |
| | | ATGACTGTCAAAGATCTCACAGCAAAATACACAGAAGGTGGAAATGCCATA |
| | | TTAGAGAACATTTCCTTCTCAATAAGTCCTGGCCAGAGGGTGGGCCTCTTG |
| | | GGAAGAACTGGATCAGGGAAGAGTACTTTGTTATCAGCTTTTTTGAGACTA |
| | | CTGAACACTGAAGGAGAAATCCAGATCGATGGTGTGTCTTGGGATTCAATA |
| | | ACTTTGCAACAGTGGAGGAAAGCCTTTGGAGTGATACCACAGAAAGTATTT |
| | | ATTTTTTCTGGAACATTTAGAAAAAACTTGGATCCCTATGAACAGTGGAGTG |
| | | ATCAAGAAATATGGAAAGTTGCAGATGAGGTTGGGCTCAGATCTGTGATA |
| | | GAACAGTTTCCTGGGAAGCTTGACTTTGTCCTTGTGGATGGGGGCTGTGT |
| | | CCTAAGCCATGGCCACAAGCAGTTGATGTGCTTGGCTAGATCTGTTCTCA |
| | | GTAAGGCGAAGATCTTGCTGCTTGATGAACCCAGTGCTCATTTGGATCCA |
| | | GTAACATACCAAATAATTAGAAGAACTCTAAAACAAGCATTTGCTGATTGCA |
| | | CAGTAATTCTCTGTGAACACAGGATAGAAGCAATGCTGGAATGCCAACAAT |
| | | TTTTGGTCATAGAAGAGAACAAAGTGCGGCAGTACGATTCCATCCAGAAAC |
| | | TGCTGAACGAGAGGAGCCTCTTCCGGCAAGCCATCAGCCCCTCCGACAG |
| | | GGTGAAGCTCTTTCCCCACCGGAACTCAAGCAAGTGCAAGTCTAAGCCCC |
| | | AGATTGCTGCTCTGAAAGAGGAGACAGAAGAAGAGGTGCAAGATACAAGG |
| | | CTTTAG |
| 8 | F5 | GAATTCGTGGTGAGCGTCTGGGCATGTCTGGGCATGTCTGGGCATGTCTG |
| | Enhancer, | GGCATGTCGGGCATTCTGGGCGTCTGGGCATGTCTGGGCATGTCTGGGC |
| | Tg83 | ATCTCGAGAACGGTGACGTGCACGCGTGGGCGGAGCCATCACGCAGGTT |
| | Promoter, | GCTATATAAGCAGAGCTCGTTTAGTGAACCGTCAGAGTCGAGCCCGAGAG |
| | 5′-UTR, | ACCATGCAGAGGTCGCCTCTGGAAAAGGCCAGCGTTGTCTCCAAACTTTT |
| | hCFTRAR, | TTTCAGCTGGACCAGACCAATTTTGAGGAAAGGATACAGACAGCGCCTGG |
| | 3′-UTR | AATTGTCAGACATATACCAAATCCCTTCTGTTGATTCTGCTGACAATCTATC |
| | | TGAAAAATTGGAAAGAGAATGGGATAGAGAGCTGGCTTCAAAGAAAAATC |
| | | CTAAACTCATTAATGCCCTTCGGCGATGTTTTTTCTGGAGATTTATGTTCTA |
| | | TGGAATCTTTTTATATTTAGGGGAAGTCACCAAAGCAGTACAGCCTCTCTT |
| | | ACTGGGAAGAATCATAGCTTCCTATGACCCGGATAACAAGGAGGAACGCT |
| | | CTATCGCGATTTATCTAGGCATAGGCTTATGCCTTCTCTTTATTGTGAGGA |
| | | CACTGCTCCTACACCCAGCCATTTTTGGCCTTCATCACATTGGAATGCAGA |
| | | TGAGAATAGCTATGTTTAGTTTGATTTATAAGAAGACTTTAAAGCTGTCAAG |
| | | CCGTGTTCTAGATAAAATAAGTATTGGACAACTTGTTAGTCTCCTTTCCAAC |
| | | AACCTGAACAAATTTGATGAAGGACTTGCATTGGCACATTTCGTGTGGATC |
| | | GCTCCTTTGCAAGTGGCACTCCTCATGGGGCTAATCTGGGAGTTGTTACA |
| | | GGCGTCTGCCTTCTGTGGACTTGGTTTCCTGATAGTCCTTGCCCTTTTTCA |
| | | GGCTGGGCTAGGGAGAATGATGATGAAGTACAGAGATCAGAGAGCTGGG |
| | | AAGATCAGTGAAAGACTTGTGATTACCTCAGAAATGATCGAGAACATCCAA |
| | | TCTGTTAAGGCATACTGCTGGGAAGAAGCAATGGAAAAAATGATTGAAAAC |
| | | TTAAGACAAACAGAACTGAAACTGACTCGGAAGGCAGCCTATGTGAGATA |
| | | CTTCAATAGCTCAGCCTTCTTCTTCTCAGGGTTCTTTGTGGTGTTTTTATCT |
| | | GTGCTTCCCTATGCACTAATCAAAGGAATCATCCTCCGGAAAATATTCACC |
| | | ACCATCTCATTCTGCATTGTTCTGCGCATGGCGGTCACTCGGCAATTTCCC |
| | | TGGGCTGTACAAACATGGTATGACTCTCTTGGAGCAATAAACAAAATACAG |
| | | GATTTCTTACAAAAGCAAGAATATAAGACATTGGAATATAACTTAACGACTA |
| | | CAGAAGTAGTGATGGAGAATGTAACAGCCTTCTGGGAGGAGGGATTTGGG |
| | | GAATTATTTGAGAAAGCAAAACAAAACAATAACAATAGAAAAACTTCTAATG |
| | | GTGATGACAGCCTCTTCTTCAGTAATTTCTCACTTCTTGGTACTCCTGTCCT |
| | | GAAAGATATTAATTTCAAGATAGAAAGAGGACAGTTGTTGGCGGTTGCTGG |
| | | ATCCACTGGAGCAGGCAAGACTTCACTTCTAATGATGATTATGGGAGAACT |
| | | GGAGCCTTCAGAGGGTAAAATTAAGCACAGTGGAAGAATTTCATTCTGTTC |
| | | TCAGTTTTCCTGGATTATGCCTGGCACCATTAAAGAAAATATCATCTTTGGT |
| | | GTTTCCTATGATGAATATAGATACAGAAGCGTCATCAAAGCATGCCAACTA |
| | | GAAGAGGACATCTCCAAGTTTGCAGAGAAAGACAATATAGTTCTTGGAGAA |
| | | GGTGGAATCACACTGAGTGGAGGTCAACGAGCAAGAATTTCTTTAGCAAG |
| | | AGCAGTATACAAAGATGCTGATTTGTATTTATTAGACTCTCCTTTTGGATAC |
| | | CTAGATGTTTTAACAGAAAAAGAAATATTTGAAAGCTGTGTCTGTAAACTGA |
| | | TGGCTAACAAAACTAGGATTTTGGTCACTTCTAAAATGGAACATTTAAAGAA |
| | | AGCTGACAAAATATTAATTTTGCATGAAGGTAGCAGCTATTTTTATGGGACA |
| | | TTTTCAGAACTCCAAAATCTACAGCCAGACTTTAGCTCAAAACTCATGGGA |
| | | TGTGATTCTTTCGACCAATTTAGTGCAGAAAGAAGAAATTCAATCCTAACTG |
| | | AGACCTTACACCGTTTCTCATTAGAAGGAGATGCTCCTGTCTCCTGGACAG |
| | | AAACAAAAAAACAATCTTTTAAACAGACTGGAGAGTTTGGGGAAAAAAGGA |
| | | AGAATTCTATTCTCAATCCAATCAACTCTACGCTTCAGGCACGAAGGAGGC |
| | | AGTCTGTCCTGAACCTGATGACACACTCAGTTAACCAAGGTCAGAACATTC |
| | | ACCGAAAGACAACAGCATCCACACGAAAAGTGTCACTGGCCCCTCAGGCA |
| | | AACTTGACTGAACTGGATATATATTCAAGAAGGTTATCTCAAGAAACTGGC |
| | | TTGGAAATAAGTGAAGAAATTAACGAAGAAGACTTAAAGGAGTGCCTTTTT |
| | | GATGATATGGAGAGCATACCAGCAGTGACTACATGGAACACATACCTTCG |
| | | ATATATTACTGTCCACAAGAGCTTAATTTTTGTGCTAATTTGGTGCTTAGTA |
| | | ATTTTTCTGGCAGAGGTGGCTGCTTCTTTGGTTGTGCTGTGGCTCCTTGGA |
| | | AACACTCCTCTTCAAGACAAAGGGAATAGTACTCATAGTAGAAATAACAGC |
| | | TATGCAGTGATTATCACCAGCACCAGTTCGTATTATGTGTTTTACATTTACG |
| | | TGGGAGTAGCCGACACTTTGCTTGCTATGGGATTCTTCAGAGGTCTACCA |
| | | CTGGTGCATACTCTAATCACAGTGTCGAAAATTTTACACCACAAAATGTTAC |
| | | ATTCTGTTCTTCAAGCACCTATGTCAACCCTCAACACGTTGAAAGCAGGTG |
| | | GGATTCTTAATAGATTCTCCAAAGATATAGCAATTTTGGATGACCTTCTGCC |
| | | TCTTACCATATTTGACTTCATCCAGTTGTTATTAATTGTGATTGGAGCTATA |
| | | GCAGTTGTCGCAGTTTTACAACCCTACATCTTTGTTGCAACAGTGCCAGTG |
| | | ATAGTGGCTTTTATTATGTTGAGAGCATATTTCCTCCAAACCTCACAGCAAC |
| | | TCAAACAACTGGAATCTGAAGGCAGGAGTCCAATTTTCACTCATCTTGTTA |
| | | CAAGCTTAAAAGGACTATGGACACTTCGTGCCTTCGGACGGCAGCCTTAC |
| | | TTTGAAACTCTGTTCCACAAAGCTCTGAATTTACATACTGCCAACTGGTTCT |
| | | TGTACCTGTCAACACTGCGCTGGTTCCAAATGAGAATAGAAATGATTTTTG |
| | | TCATCTTCTTCATTGCTGTTACCTTCATTTCCATTTTAACAACAGGAGAAGG |
| | | AGAAGGAAGAGTTGGTATTATCCTGACTTTAGCCATGAATATCATGAGTAC |
| | | ATTGCAGTGGGCTGTAAACTCCAGCATAGATGTGGATAGCTTGATGCGAT |
| | | CTGTGAGCCGAGTCTTTAAGTTCATTGACATGCCAACAGAAGGTAAACCTA |
| | | CCAAGTCAACCAAACCATACAAGAATGGCCAACTCTCGAAAGTTATGATTA |
| | | TTGAGAATTCACACGTGAAGAAAGATGACATCTGGCCCTCAGGGGGCCAA |
| | | ATGACTGTCAAAGATCTCACAGCAAAATACACAGAAGGTGGAAATGCCATA |
| | | TTAGAGAACATTTCCTTCTCAATAAGTCCTGGCCAGAGGGTGGGCCTCTTG |
| | | GGAAGAACTGGATCAGGGAAGAGTACTTTGTTATCAGCTTTTTTGAGACTA |
| | | CTGAACACTGAAGGAGAAATCCAGATCGATGGTGTGTCTTGGGATTCAATA |
| | | ACTTTGCAACAGTGGAGGAAAGCCTTTGGAGTGATACCACAGAAAGTATTT |
| | | ATTTTTTCTGGAACATTTAGAAAAAACTTGGATCCCTATGAACAGTGGAGTG |
| | | ATCAAGAAATATGGAAAGTTGCAGATGAGGTTGGGCTCAGATCTGTGATA |
| | | GAACAGTTTCCTGGGAAGCTTGACTTTGTCCTTGTGGATGGGGGCTGTGT |
| | | CCTAAGCCATGGCCACAAGCAGTTGATGTGCTTGGCTAGATCTGTTCTCA |
| | | GTAAGGCGAAGATCTTGCTGCTTGATGAACCCAGTGCTCATTTGGATCCA |
| | | GTAACATACCAAATAATTAGAAGAACTCTAAAACAAGCATTTGCTGATTGCA |
| | | CAGTAATTCTCTGTGAACACAGGATAGAAGCAATGCTGGAATGCCAACAAT |
| | | TTTTGGTCATAGAAGAGAACAAAGTGCGGCAGTACGATTCCATCCAGAAAC |
| | | TGCTGAACGAGAGGAGCCTCTTCCGGCAAGCCATCAGCCCCTCCGACAG |
| | | GGTGAAGCTCTTTCCCCACCGGAACTCAAGCAAGTGCAAGTCTAAGCCCC |
| | | AGATTGCTGCTCTGAAAGAGGAGACAGAAGAAGAGGTGCAAGATACAAGG |
| | | CTTTAGAGAGCAGCATAAATGTTGACATGGGACATTTGCTCATGGAATTGG |
| 9 | 5′ AAV ITR | TTGGCCACTCCCTCTCTGCGCGCTCGCTCGCTCACTGAGGCCGGGCGAC |
| | | CAAAGGTCGCCCGACGCCCGGGCTTTGCCCGGGGGGCCTCAGTGAGCGA |
| | | GCGAGCGCGCAGAGAGGGAGTGGCCAACTCCATCACTAGGGGTTCCT |
| 10 | 3′ AAV ITR | AGGAACCCCTAGTGATGGAGTTGGCCACTCCCTCTCTGCGCGCTCGCTCG |
| | | CTCACTGAGGCCGCCCGGGCAAAGCCCGGGCGTCGGGCGACCTTTGGTC |
| | | GCCCGGCCTCAGTGAGCGAGCGAGCGCGCAGAGAGGGAGTGGCCAA |
| 11 | 5′ AAV ITR | TTGGCCACTCCCTCTCTGCGCGCTCGCTCGCTCACTGAGGCCGGGCGAC |
| | through 3′ | CAAAGGTCGCCCGACGCCCGGGCTTTGCCCGGGGGGCCTCAGTGAGCGA |
| | ITR | GCGAGCGCGCAGAGAGGGAGTGGCCAACTCCATCACTAGGGGTTCCTCA |
| | | GATCTGAATTCGTGGTGAGCGTCTGGGCATGTCTGGGCATGTCTGGGCAT |
| | | GTCTGGGCATGTCGGGCATTCTGGGCGTCTGGGCATGTCTGGGCATGTCT |
| | | GGGCATCTCGAGAACGGTGACGTGCACGCGTGGGCGGAGCCATCACGCA |
| | | GGTTGCTATATAAGCAGAGCTCGTTTAGTGAACCGTCAGAGTCGAGCCCG |
| | | AGAGACCATGCAGAGGTCGCCTCTGGAAAAGGCCAGCGTTGTCTCCAAAC |
| | | TTTTTTTCAGCTGGACCAGACCAATTTTGAGGAAAGGATACAGACAGCGCC |
| | | TGGAATTGTCAGACATATACCAAATCCCTTCTGTTGATTCTGCTGACAATCT |
| | | ATCTGAAAAATTGGAAAGAGAATGGGATAGAGAGCTGGCTTCAAAGAAAAA |
| | | TCCTAAACTCATTAATGCCCTTCGGCGATGTTTTTTCTGGAGATTTATGTTC |
| | | TATGGAATCTTTTTATATTTAGGGGAAGTCACCAAAGCAGTACAGCCTCTC |
| | | TTACTGGGAAGAATCATAGCTTCCTATGACCCGGATAACAAGGAGGAACG |
| | | CTCTATCGCGATTTATCTAGGCATAGGCTTATGCCTTCTCTTTATTGTGAGG |
| | | ACACTGCTCCTACACCCAGCCATTTTTGGCCTTCATCACATTGGAATGCAG |
| | | ATGAGAATAGCTATGTTTAGTTTGATTTATAAGAAGACTTTAAAGCTGTCAA |
| | | GCCGTGTTCTAGATAAAATAAGTATTGGACAACTTGTTAGTCTCCTTTCCAA |
| | | CAACCTGAACAAATTTGATGAAGGACTTGCATTGGCACATTTCGTGTGGAT |
| | | CGCTCCTTTGCAAGTGGCACTCCTCATGGGGCTAATCTGGGAGTTGTTAC |
| | | AGGCGTCTGCCTTCTGTGGACTTGGTTTCCTGATAGTCCTTGCCCTTTTTC |
| | | AGGCTGGGCTAGGGAGAATGATGATGAAGTACAGAGATCAGAGAGCTGG |
| | | GAAGATCAGTGAAAGACTTGTGATTACCTCAGAAATGATCGAGAACATCCA |
| | | ATCTGTTAAGGCATACTGCTGGGAAGAAGCAATGGAAAAAATGATTGAAAA |
| | | CTTAAGACAAACAGAACTGAAACTGACTCGGAAGGCAGCCTATGTGAGAT |
| | | ACTTCAATAGCTCAGCCTTCTTCTTCTCAGGGTTCTTTGTGGTGTTTTTATC |
| | | TGTGCTTCCCTATGCACTAATCAAAGGAATCATCCTCCGGAAAATATTCAC |
| | | CACCATCTCATTCTGCATTGTTCTGCGCATGGCGGTCACTCGGCAATTTCC |
| | | CTGGGCTGTACAAACATGGTATGACTCTCTTGGAGCAATAAACAAAATACA |
| | | GGATTTCTTACAAAAGCAAGAATATAAGACATTGGAATATAACTTAACGACT |
| | | ACAGAAGTAGTGATGGAGAATGTAACAGCCTTCTGGGAGGAGGGATTTGG |
| | | GGAATTATTTGAGAAAGCAAAACAAAACAATAACAATAGAAAAACTTCTAAT |
| | | GGTGATGACAGCCTCTTCTTCAGTAATTTCTCACTTCTTGGTACTCCTGTC |
| | | CTGAAAGATATTAATTTCAAGATAGAAAGAGGACAGTTGTTGGCGGTTGCT |
| | | GGATCCACTGGAGCAGGCAAGACTTCACTTCTAATGATGATTATGGGAGA |
| | | ACTGGAGCCTTCAGAGGGTAAAATTAAGCACAGTGGAAGAATTTCATTCTG |
| | | TTCTCAGTTTTCCTGGATTATGCCTGGCACCATTAAAGAAAATATCATCTTT |
| | | GGTGTTTCCTATGATGAATATAGATACAGAAGCGTCATCAAAGCATGCCAA |
| | | CTAGAAGAGGACATCTCCAAGTTTGCAGAGAAAGACAATATAGTTCTTGGA |
| | | GAAGGTGGAATCACACTGAGTGGAGGTCAACGAGCAAGAATTTCTTTAGC |
| | | AAGAGCAGTATACAAAGATGCTGATTTGTATTTATTAGACTCTCCTTTTGGA |
| | | TACCTAGATGTTTTAACAGAAAAAGAAATATTTGAAAGCTGTGTCTGTAAAC |
| | | TGATGGCTAACAAAACTAGGATTTTGGTCACTTCTAAAATGGAACATTTAAA |
| | | GAAAGCTGACAAAATATTAATTTTGCATGAAGGTAGCAGCTATTTTTATGGG |
| | | ACATTTTCAGAACTCCAAAATCTACAGCCAGACTTTAGCTCAAAACTCATG |
| | | GGATGTGATTCTTTCGACCAATTTAGTGCAGAAAGAAGAAATTCAATCCTA |
| | | ACTGAGACCTTACACCGTTTCTCATTAGAAGGAGATGCTCCTGTCTCCTGG |
| | | ACAGAAACAAAAAAACAATCTTTTAAACAGACTGGAGAGTTTGGGGAAAAA |
| | | AGGAAGAATTCTATTCTCAATCCAATCAACTCTACGCTTCAGGCACGAAGG |
| | | AGGCAGTCTGTCCTGAACCTGATGACACACTCAGTTAACCAAGGTCAGAA |
| | | CATTCACCGAAAGACAACAGCATCCACACGAAAAGTGTCACTGGCCCCTC |
| | | AGGCAAACTTGACTGAACTGGATATATATTCAAGAAGGTTATCTCAAGAAA |
| | | CTGGCTTGGAAATAAGTGAAGAAATTAACGAAGAAGACTTAAAGGAGTGC |
| | | CTTTTTGATGATATGGAGAGCATACCAGCAGTGACTACATGGAACACATAC |
| | | CTTCGATATATTACTGTCCACAAGAGCTTAATTTTTGTGCTAATTTGGTGCT |
| | | TAGTAATTTTTCTGGCAGAGGTGGCTGCTTCTTTGGTTGTGCTGTGGCTCC |
| | | TTGGAAACACTCCTCTTCAAGACAAAGGGAATAGTACTCATAGTAGAAATA |
| | | ACAGCTATGCAGTGATTATCACCAGCACCAGTTCGTATTATGTGTTTTACAT |
| | | TTACGTGGGAGTAGCCGACACTTTGCTTGCTATGGGATTCTTCAGAGGTCT |
| | | ACCACTGGTGCATACTCTAATCACAGTGTCGAAAATTTTACACCACAAAAT |
| | | GTTACATTCTGTTCTTCAAGCACCTATGTCAACCCTCAACACGTTGAAAGC |
| | | AGGTGGGATTCTTAATAGATTCTCCAAAGATATAGCAATTTTGGATGACCTT |
| | | CTGCCTCTTACCATATTTGACTTCATCCAGTTGTTATTAATTGTGATTGGAG |
| | | CTATAGCAGTTGTCGCAGTTTTACAACCCTACATCTTTGTTGCAACAGTGC |
| | | CAGTGATAGTGGCTTTTATTATGTTGAGAGCATATTTCCTCCAAACCTCACA |
| | | GCAACTCAAACAACTGGAATCTGAAGGCAGGAGTCCAATTTTCACTCATCT |
| | | TGTTACAAGCTTAAAAGGACTATGGACACTTCGTGCCTTCGGACGGCAGC |
| | | CTTACTTTGAAACTCTGTTCCACAAAGCTCTGAATTTACATACTGCCAACTG |
| | | GTTCTTGTACCTGTCAACACTGCGCTGGTTCCAAATGAGAATAGAAATGAT |
| | | TTTTGTCATCTTCTTCATTGCTGTTACCTTCATTTCCATTTTAACAACAGGAG |
| | | AAGGAGAAGGAAGAGTTGGTATTATCCTGACTTTAGCCATGAATATCATGA |
| | | GTACATTGCAGTGGGCTGTAAACTCCAGCATAGATGTGGATAGCTTGATG |
| | | CGATCTGTGAGCCGAGTCTTTAAGTTCATTGACATGCCAACAGAAGGTAAA |
| | | CCTACCAAGTCAACCAAACCATACAAGAATGGCCAACTCTCGAAAGTTATG |
| | | ATTATTGAGAATTCACACGTGAAGAAAGATGACATCTGGCCCTCAGGGGG |
| | | CCAAATGACTGTCAAAGATCTCACAGCAAAATACACAGAAGGTGGAAATGC |
| | | CATATTAGAGAACATTTCCTTCTCAATAAGTCCTGGCCAGAGGGTGGGCCT |
| | | CTTGGGAAGAACTGGATCAGGGAAGAGTACTTTGTTATCAGCTTTTTTGAG |
| | | ACTACTGAACACTGAAGGAGAAATCCAGATCGATGGTGTGTCTTGGGATTC |
| | | AATAACTTTGCAACAGTGGAGGAAAGCCTTTGGAGTGATACCACAGAAAGT |
| | | ATTTATTTTTTCTGGAACATTTAGAAAAAACTTGGATCCCTATGAACAGTGG |
| | | AGTGATCAAGAAATATGGAAAGTTGCAGATGAGGTTGGGCTCAGATCTGT |
| | | GATAGAACAGTTTCCTGGGAAGCTTGACTTTGTCCTTGTGGATGGGGGCT |
| | | GTGTCCTAAGCCATGGCCACAAGCAGTTGATGTGCTTGGCTAGATCTGTT |
| | | CTCAGTAAGGCGAAGATCTTGCTGCTTGATGAACCCAGTGCTCATTTGGAT |
| | | CCAGTAACATACCAAATAATTAGAAGAACTCTAAAACAAGCATTTGCTGATT |
| | | GCACAGTAATTCTCTGTGAACACAGGATAGAAGCAATGCTGGAATGCCAA |
| | | CAATTTTTGGTCATAGAAGAGAACAAAGTGCGGCAGTACGATTCCATCCAG |
| | | AAACTGCTGAACGAGAGGAGCCTCTTCCGGCAAGCCATCAGCCCCTCCGA |
| | | CAGGGTGAAGCTCTTTCCCCACCGGAACTCAAGCAAGTGCAAGTCTAAGC |
| | | CCCAGATTGCTGCTCTGAAAGAGGAGACAGAAGAAGAGGTGCAAGATACA |
| | | AGGCTTTAGAGAGCAGCATAAATGTTGACATGGGACATTTGCTCATGGAAT |
| | | TGGCAGGCCTAATAAAGAGCTCAGATGCATCGATCAGAGTGTGTTGGTTTT |
| | | TTGTGTGTACTGAGGAACCCCTAGTGATGGAGTTGGCCACTCCCTCTCTG |
| | | CGCGCTCGCTCGCTCACTGAGGCCGCCCGGGCAAAGCCCGGGCGTCGG |
| | | GCGACCTTTGGTCGCCCGGCCTCAGTGAGCGAGCGAGCGCGCAGAGAG |
| | | GGAGTGGCCAA |
| 12 | pAV- | TTGGCCACTCCCTCTCTGCGCGCTCGCTCGCTCACTGAGGCCGGGCGAC |
| | F5tg83- | CAAAGGTCGCCCGACGCCCGGGCTTTGCCCGGGGGGCCTCAGTGAGCGA |
| | hCFTR-dR | GCGAGCGCGCAGAGAGGGAGTGGCCAACTCCATCACTAGGGGTTCCTCA |
| | vector | GATCTGAATTCGTGGTGAGCGTCTGGGCATGTCTGGGCATGTCTGGGCAT |
| | | GTCTGGGCATGTCGGGCATTCTGGGCGTCTGGGCATGTCTGGGCATGTCT |
| | | GGGCATCTCGAGAACGGTGACGTGCACGCGTGGGCGGAGCCATCACGCA |
| | | GGTTGCTATATAAGCAGAGCTCGTTTAGTGAACCGTCAGAGTCGAGCCCG |
| | | AGAGACCATGCAGAGGTCGCCTCTGGAAAAGGCCAGCGTTGTCTCCAAAC |
| | | TTTTTTTCAGCTGGACCAGACCAATTTTGAGGAAAGGATACAGACAGCGCC |
| | | TGGAATTGTCAGACATATACCAAATCCCTTCTGTTGATTCTGCTGACAATCT |
| | | ATCTGAAAAATTGGAAAGAGAATGGGATAGAGAGCTGGCTTCAAAGAAAAA |
| | | TCCTAAACTCATTAATGCCCTTCGGCGATGTTTTTTCTGGAGATTTATGTTC |
| | | TATGGAATCTTTTTATATTTAGGGGAAGTCACCAAAGCAGTACAGCCTCTC |
| | | TTACTGGGAAGAATCATAGCTTCCTATGACCCGGATAACAAGGAGGAACG |
| | | CTCTATCGCGATTTATCTAGGCATAGGCTTATGCCTTCTCTTTATTGTGAGG |
| | | ACACTGCTCCTACACCCAGCCATTTTTGGCCTTCATCACATTGGAATGCAG |
| | | ATGAGAATAGCTATGTTTAGTTTGATTTATAAGAAGACTTTAAAGCTGTCAA |
| | | GCCGTGTTCTAGATAAAATAAGTATTGGACAACTTGTTAGTCTCCTTTCCAA |
| | | CAACCTGAACAAATTTGATGAAGGACTTGCATTGGCACATTTCGTGTGGAT |
| | | CGCTCCTTTGCAAGTGGCACTCCTCATGGGGCTAATCTGGGAGTTGTTAC |
| | | AGGCGTCTGCCTTCTGTGGACTTGGTTTCCTGATAGTCCTTGCCCTTTTTC |
| | | AGGCTGGGCTAGGGAGAATGATGATGAAGTACAGAGATCAGAGAGCTGG |
| | | GAAGATCAGTGAAAGACTTGTGATTACCTCAGAAATGATCGAGAACATCCA |
| | | ATCTGTTAAGGCATACTGCTGGGAAGAAGCAATGGAAAAAATGATTGAAAA |
| | | CTTAAGACAAACAGAACTGAAACTGACTCGGAAGGCAGCCTATGTGAGAT |
| | | ACTTCAATAGCTCAGCCTTCTTCTTCTCAGGGTTCTTTGTGGTGTTTTTATC |
| | | TGTGCTTCCCTATGCACTAATCAAAGGAATCATCCTCCGGAAAATATTCAC |
| | | CACCATCTCATTCTGCATTGTTCTGCGCATGGCGGTCACTCGGCAATTTCC |
| | | CTGGGCTGTACAAACATGGTATGACTCTCTTGGAGCAATAAACAAAATACA |
| | | GGATTTCTTACAAAAGCAAGAATATAAGACATTGGAATATAACTTAACGACT |
| | | ACAGAAGTAGTGATGGAGAATGTAACAGCCTTCTGGGAGGAGGGATTTGG |
| | | GGAATTATTTGAGAAAGCAAAAAAAACAATAACAATAGAAAAACTTCTAAT |
| | | GGTGATGACAGCCTCTTCTTCAGTAATTTCTCACTTCTTGGTACTCCTGTC |
| | | CTGAAAGATATTAATTTCAAGATAGAAAGAGGACAGTTGTTGGCGGTTGCT |
| | | GGATCCACTGGAGCAGGCAAGACTTCACTTCTAATGATGATTATGGGAGA |
| | | ACTGGAGCCTTCAGAGGGTAAAATTAAGCACAGTGGAAGAATTTCATTCTG |
| | | TTCTCAGTTTTCCTGGATTATGCCTGGCACCATTAAAGAAAATATCATCTTT |
| | | GGTGTTTCCTATGATGAATATAGATACAGAAGCGTCATCAAAGCATGCCAA |
| | | CTAGAAGAGGACATCTCCAAGTTTGCAGAGAAAGACAATATAGTTCTTGGA |
| | | GAAGGTGGAATCACACTGAGTGGAGGTCAACGAGCAAGAATTTCTTTAGC |
| | | AAGAGCAGTATACAAAGATGCTGATTTGTATTTATTAGACTCTCCTTTTGGA |
| | | TACCTAGATGTTTTAACAGAAAAAGAAATATTTGAAAGCTGTGTCTGTAAAC |
| | | TGATGGCTAACAAAACTAGGATTTTGGTCACTTCTAAAATGGAACATTTAAA |
| | | GAAAGCTGACAAAATATTAATTTTGCATGAAGGTAGCAGCTATTTTTATGGG |
| | | ACATTTTCAGAACTCCAAAATCTACAGCCAGACTTTAGCTCAAAACTCATG |
| | | GGATGTGATTCTTTCGACCAATTTAGTGCAGAAAGAAGAAATTCAATCCTA |
| | | ACTGAGACCTTACACCGTTTCTCATTAGAAGGAGATGCTCCTGTCTCCTGG |
| | | ACAGAAACAAAAAAACAATCTTTTAAACAGACTGGAGAGTTTGGGGAAAAA |
| | | AGGAAGAATTCTATTCTCAATCCAATCAACTCTACGCTTCAGGCACGAAGG |
| | | AGGCAGTCTGTCCTGAACCTGATGACACACTCAGTTAACCAAGGTCAGAA |
| | | CATTCACCGAAAGACAACAGCATCCACACGAAAAGTGTCACTGGCCCCTC |
| | | AGGCAAACTTGACTGAACTGGATATATATTCAAGAAGGTTATCTCAAGAAA |
| | | CTGGCTTGGAAATAAGTGAAGAAATTAACGAAGAAGACTTAAAGGAGTGC |
| | | CTTTTTGATGATATGGAGAGCATACCAGCAGTGACTACATGGAACACATAC |
| | | CTTCGATATATTACTGTCCACAAGAGCTTAATTTTTGTGCTAATTTGGTGCT |
| | | TAGTAATTTTTCTGGCAGAGGTGGCTGCTTCTTTGGTTGTGCTGTGGCTCC |
| | | TTGGAAACACTCCTCTTCAAGACAAAGGGAATAGTACTCATAGTAGAAATA |
| | | ACAGCTATGCAGTGATTATCACCAGCACCAGTTCGTATTATGTGTTTTACAT |
| | | TTACGTGGGAGTAGCCGACACTTTGCTTGCTATGGGATTCTTCAGAGGTCT |
| | | ACCACTGGTGCATACTCTAATCACAGTGTCGAAAATTTTACACCACAAAAT |
| | | GTTACATTCTGTTCTTCAAGCACCTATGTCAACCCTCAACACGTTGAAAGC |
| | | AGGTGGGATTCTTAATAGATTCTCCAAAGATATAGCAATTTTGGATGACCTT |
| | | CTGCCTCTTACCATATTTGACTTCATCCAGTTGTTATTAATTGTGATTGGAG |
| | | CTATAGCAGTTGTCGCAGTTTTACAACCCTACATCTTTGTTGCAACAGTGC |
| | | CAGTGATAGTGGCTTTTATTATGTTGAGAGCATATTTCCTCCAAACCTCACA |
| | | GCAACTCAAACAACTGGAATCTGAAGGCAGGAGTCCAATTTTCACTCATCT |
| | | TGTTACAAGCTTAAAAGGACTATGGACACTTCGTGCCTTCGGACGGCAGC |
| | | CTTACTTTGAAACTCTGTTCCACAAAGCTCTGAATTTACATACTGCCAACTG |
| | | GTTCTTGTACCTGTCAACACTGCGCTGGTTCCAAATGAGAATAGAAATGAT |
| | | TTTTGTCATCTTCTTCATTGCTGTTACCTTCATTTCCATTTTAACAACAGGAG |
| | | AAGGAGAAGGAAGAGTTGGTATTATCCTGACTTTAGCCATGAATATCATGA |
| | | GTACATTGCAGTGGGCTGTAAACTCCAGCATAGATGTGGATAGCTTGATG |
| | | CGATCTGTGAGCCGAGTCTTTAAGTTCATTGACATGCCAACAGAAGGTAAA |
| | | CCTACCAAGTCAACCAAACCATACAAGAATGGCCAACTCTCGAAAGTTATG |
| | | ATTATTGAGAATTCACACGTGAAGAAAGATGACATCTGGCCCTCAGGGGG |
| | | CCAAATGACTGTCAAAGATCTCACAGCAAAATACACAGAAGGTGGAAATGC |
| | | CATATTAGAGAACATTTCCTTCTCAATAAGTCCTGGCCAGAGGGTGGGCCT |
| | | CTTGGGAAGAACTGGATCAGGGAAGAGTACTTTGTTATCAGCTTTTTTGAG |
| | | ACTACTGAACACTGAAGGAGAAATCCAGATCGATGGTGTGTCTTGGGATTC |
| | | AATAACTTTGCAACAGTGGAGGAAAGCCTTTGGAGTGATACCACAGAAAGT |
| | | ATTTATTTTTTCTGGAACATTTAGAAAAAACTTGGATCCCTATGAACAGTGG |
| | | AGTGATCAAGAAATATGGAAAGTTGCAGATGAGGTTGGGCTCAGATCTGT |
| | | GATAGAACAGTTTCCTGGGAAGCTTGACTTTGTCCTTGTGGATGGGGGCT |
| | | GTGTCCTAAGCCATGGCCACAAGCAGTTGATGTGCTTGGCTAGATCTGTT |
| | | CTCAGTAAGGCGAAGATCTTGCTGCTTGATGAACCCAGTGCTCATTTGGAT |
| | | CCAGTAACATACCAAATAATTAGAAGAACTCTAAAACAAGCATTTGCTGATT |
| | | GCACAGTAATTCTCTGTGAACACAGGATAGAAGCAATGCTGGAATGCCAA |
| | | CAATTTTTGGTCATAGAAGAGAACAAAGTGCGGCAGTACGATTCCATCCAG |
| | | AAACTGCTGAACGAGAGGAGCCTCTTCCGGCAAGCCATCAGCCCCTCCGA |
| | | CAGGGTGAAGCTCTTTCCCCACCGGAACTCAAGCAAGTGCAAGTCTAAGC |
| | | CCCAGATTGCTGCTCTGAAAGAGGAGACAGAAGAAGAGGTGCAAGATACA |
| | | AGGCTTTAGAGAGCAGCATAAATGTTGACATGGGACATTTGCTCATGGAAT |
| | | TGGCAGGCCTAATAAAGAGCTCAGATGCATCGATCAGAGTGTGTTGGTTTT |
| | | TTGTGTGTACTGAGGAACCCCTAGTGATGGAGTTGGCCACTCCCTCTCTG |
| | | CGCGCTCGCTCGCTCACTGAGGCCGCCCGGGCAAAGCCCGGGCGTCGG |
| | | GCGACCTTTGGTCGCCCGGCCTCAGTGAGCGAGCGAGCGCGCAGAGAG |
| | | GGAGTGGCCAACCCCCCCCCCCCCCCCCCTGCAGCCAGCTGGCGTAATA |
| | | GCGAAGAGGCCCGCACCGATCGCCCTTCCCAACAGTTGCGTAGCCTGAAT |
| | | GGCGAATGGCGCGACGCGCCCTGTAGCGGCGCATTAAGCGCGGCGGGT |
| | | GTGGTGGTTACGCGCAGCGTGACCGCTACACTTGCCAGCGCCCTAGCGC |
| | | CCGCTCCTTTCGCTTTCTTCCCTTCCTTTCTCGCCACGTTCGCCGGCTTTC |
| | | CCCGTCAAGCTCTAAATCGGGGGCTCCCTTTAGGGTTCCGATTTAGTGCTT |
| | | TACGGCACCTCGACCCCAAAAAACTTGATTAGGGTGATGGTTCACGTAGT |
| | | GGGCCATCGCCCTGATAGACGGTTTTTCGCCCTTTGACGTTGGAGTCCAC |
| | | GTTCTTTAATAGTGGACTCTTGTTCCAAACTGGAACAACACTCAACCCTATC |
| | | TCGGTCTATTCTTTTGATTTATAAGGGATTTTGCCGATTTCGGCCTATTGGT |
| | | TAAAAAATGAGCTGATTTAACAAAAATTTAACGCGAATTTTAACAAAATATTA |
| | | ACGTTTACAATTTCCTGATGCGGTATTTTCTCCTTACGCATCTGTGCGGTAT |
| | | TTCACACCGCATATGGTGCACTCTCAGTACAATCTGCTCTGATGCCGCATA |
| | | GTTAAGCCAGCCCCGACACCCGCCAACACCCGCTGACGCGCCCTGACGG |
| | | GCTTGTCTGCTCCCGGCATCCGCTTACAGACAAGCTGTGACCGTCTCCGG |
| | | GAGCTGCATGTGTCAGAGGTTTTCACCGTCATCACCGAAACGCGCGAGAC |
| | | GAAAGGGCCTCGTGATACGCCTATTTTTATAGGTTAATGTCATGATAATAAT |
| | | GGTTTCTTAGACGTCAGGTGGCACTTTTCGGGGAAATGTGCGCGGAACCC |
| | | CTATTTGTTTATTTTTCTAAATACATTCAAATATGTATCCGCTCATGAGACAA |
| | | TAACCCTGATAAATGCTTCAATAATATTGAAAAAGGAAGAGTATGAGTATTC |
| | | AACATTTCCGTGTCGCCCTTATTCCCTTTTTTGCGGCATTTTGCCTTCCTGT |
| | | TTTTGCTCACCCAGAAACGCTGGTGAAAGTAAAAGATGCTGAAGATCAGTT |
| | | GGGTGCACGAGTGGGTTACATCGAACTGGATCTCAACAGCGGTAAGATCC |
| | | TTGAGAGTTTTCGCCCCGAAGAACGTTTTCCAATGATGAGCACTTTTAAAG |
| | | TTCTGCTATGTGGCGCGGTATTATCCCGTATTGACGCCGGGCAAGAGCAA |
| | | CTCGGTCGCCGCATACACTATTCTCAGAATGACTTGGTTGAGTACTCACCA |
| | | GTCACAGAAAAGCATCTTACGGATGGCATGACAGTAAGAGAATTATGCAGT |
| | | GCTGCCATAACCATGAGTGATAACACTGCGGCCAACTTACTTCTGACAACG |
| | | ATCGGAGGACCGAAGGAGCTAACCGCTTTTTTGCACAACATGGGGGATCA |
| | | TGTAACTCGCCTTGATCGTTGGGAACCGGAGCTGAATGAAGCCATACCAA |
| | | ACGACGAGCGTGACACCACGATGCCTGTAGCAATGGCAACAACGTTGCG |
| | | CAAACTATTAACTGGCGAACTACTTACTCTAGCTTCCCGGCAACAATTAATA |
| | | GACTGGATGGAGGCGGATAAAGTTGCAGGACCACTTCTGCGCTCGGCCC |
| | | TTCCGGCTGGCTGGTTTATTGCTGATAAATCTGGAGCCGGTGAGCGTGGG |
| | | TCTCGCGGTATCATTGCAGCACTGGGGCCAGATGGTAAGCCCTCCCGTAT |
| | | CGTAGTTATCTACACGACGGGGAGTCAGGCAACTATGGATGAACGAAATA |
| | | GACAGATCGCTGAGATAGGTGCCTCACTGATTAAGCATTGGTAACTGTCA |
| | | GACCAAGTTTACTCATATATACTTTAGATTGATTTAAAACTTCATTTTTAATT |
| | | TAAAAGGATCTAGGTGAAGATCCTTTTTGATAATCTCATGACCAAAATCCCT |
| | | TAACGTGAGTTTTCGTTCCACTGAGCGTCAGACCCCGTAGAAAAGATCAAA |
| | | GGATCTTCTTGAGATCCTTTTTTTCTGCGCGTAATCTGCTGCTTGCAAACAA |
| | | AAAAACCACCGCTACCAGCGGTGGTTTGTTTGCCGGATCAAGAGCTACCA |
| | | ACTCTTTTTCCGAAGGTAACTGGCTTCAGCAGAGCGCAGATACCAAATACT |
| | | GTCCTTCTAGTGTAGCCGTAGTTAGGCCACCACTTCAAGAACTCTGTAGCA |
| | | CCGCCTACATACCTCGCTCTGCTAATCCTGTTACCAGTGGCTGCTGCCAG |
| | | TGGCGATAAGTCGTGTCTTACCGGGTTGGACTCAAGACGATAGTTACCGG |
| | | ATAAGGCGCAGCGGTCGGGCTGAACGGGGGGTTCGTGCACACAGCCCAG |
| | | CTTGGAGCGAACGACCTACACCGAACTGAGATACCTACAGCGTGAGCATT |
| | | GAGAAAGCGCCACGCTTCCCGAAGGGAGAAAGGCGGACAGGTATCCGGT |
| | | AAGCGGCAGGGTCGGAACAGGAGAGCGCACGAGGGAGCTTCCAGGGGG |
| | | AAACGCCTGGTATCTTTATAGTCCTGTCGGGTTTCGCCACCTCTGACTTGA |
| | | GCGTCGATTTTTGTGATGCTCGTCAGGGGGGCGGAGCCTATGGAAAAACG |
| | | CCAGCAACGCGGCCTTTTTACGGTTCCTGGCCTTTTGCTGGCCTTTTGCTC |
| | | ACATGTTCTTTCCTGCGTTATCCCCTGATTCTGTGGATAACCGTATTACCG |
| | | CCTTTGAGTGAGCTGATACCGCTCGCCGCAGCCGAACGACCGAGCGCAG |
| | | CGAGTCAGTGAGCGAGGAAGCGGAAGAGCGCCCAATACGCAAACCGCCT |
| | | CTCCCCGCGCGTTGGCCGATTCATTAATGCAGCTGGGCTGCAGGGGGGG |
| | | GGGGGGGGGGG |
| 13 | AV.TL65 | MAADGYLPDWLEDTLSEGIRQWWKLKPGPPPPKPAERHKDDSRGLVLPGYK |
| | capsid | YLGPFNGLDKGEPVNEADAAALEHDKAYDRQLDSGDNPYLKYNHADAEFQE |
| | protein | RLKEDTSFGGNLGRAVFQAKKRVLEPFGLVEEGAKTAPTGKRIDDHFPKRKK |
| | | ARTEEDSKPSTSSDAEAGPSGSQQLQIPAQPASSLGADTMSAGGGGPLGDN |
| | | NQGADGVGNASGDWHCDSTWMGDRVVTKSTRTWVLPSYNNHQYREIKSGS |
| | | VDGSNANAYFGYSTPWGYFDFNRFHSHWSPRDWQRLINNYWGFRPRSLRV |
| | | KIFNIQVKEVTVQDSTTTIANNLTSTVQVFTDDDYQLPYVVGNGTEGCLPAFPP |
| | | QVFTLPQYGYATLNRDNTENPTERSSFFCLEYFPSKMLRTGNNFEFTYNFEE |
| | | VPFHSSFAPSQNLFKLANPLVDQYLYRFVSTNNTGGVQFNKNLAGRYANTYK |
| | | NWFPGPMGRTQGWNLGSGVNRASVSAFATTNRMELEGASYQVPPQPNGM |
| | | TNNLQGSNTYALENTMIFNSQPANPGTTATYLEGNMLITSESETQPVNRVAYN |
| | | VGGQMATNNQSSTTAPTTGTYNLQEIVPGSVWMERDVYLQGPIWAKIPETGA |
| | | HFHPSPAMGGFGLKHPPPMMLIKNTPVPGNITSFSDVPVSSFITQYSTGQVTV |
| | | EMEWELKKENSKRWNPEIQYTNNYNDPQFVDFAPDSTGEYRTTRPIGTRYLT |
| | | RPL |
| 14 | F5 | GTGGTGAGCGTCTGGGCATGTCTGGGCATGTCTGGGCATGTCTGGGCAT |
| | enhancer | GTCGGGCATTCTGGGCGTCTGGGCATGTCTGGGCATGTCTGGGCAT |
| 15 | 5′ AAV ITR | CCACTCCCTCTCTGCGCGCTCGCTCGCTCACTGAGGCCGCCCGGGCAAA |
| | (flop) | GCCCGGGCGTCGGGCGACCTTTGGTCGCCCGGCCTCAGTGAGCGAGCG |
| | | AGCGCGCAGAGAGGGAGTGGCCAACTCCATCACTAGGGGTTCCT |
| 16 | 3′ AAV ITR | AGGAACCCCTAGTGATGGAGTTGGCCACTCCCTCTCTGCGCGCTCGCTCG |
| | (flop) | CTCACTGAGGCCGGGCGACCAAAGGTCGCCCGACGCCCGGGCTTTGCCC |
| | | GGGGGGCCTCAGTGAGCGAGCGAGCGCGCAGAGAGGGAGTGGCC |
| 17 | 5′ AAV ITR | CCACTCCCTCTCTGCGCGCTCGCTCGCTCACTGAGGCCGCCCGGGCAAA |
| | (flop) | GCCCGGGCGTCGGGCGACCTTTGGTCGCCCGGCCTCAGTGAGCGAGCG |
| | through 3′ | AGCGCGCAGAGAGGGAGTGGCCAACTCCATCACTAGGGGTTCCTCAGAT |
| | AAV ITR | CTGAATTCGTGGTGAGCGTCTGGGCATGTCTGGGCATGTCTGGGCATGTC |
| | (flop) | TGGGCATGTCGGGCATTCTGGGCGTCTGGGCATGTCTGGGCATGTCTGG |
| | | GCATCTCGAGAACGGTGACGTGCACGCGTGGGCGGAGCCATCACGCAGG |
| | | TTGCTATATAAGCAGAGCTCGTTTAGTGAACCGTCAGAGTCGAGCCCGAG |
| | | AGACCATGCAGAGGTCGCCTCTGGAAAAGGCCAGCGTTGTCTCCAAACTT |
| | | TTTTTCAGCTGGACCAGACCAATTTTGAGGAAAGGATACAGACAGCGCCT |
| | | GGAATTGTCAGACATATACCAAATCCCTTCTGTTGATTCTGCTGACAATCTA |
| | | TCTGAAAAATTGGAAAGAGAATGGGATAGAGAGCTGGCTTCAAAGAAAAAT |
| | | CCTAAACTCATTAATGCCCTTCGGCGATGTTTTTTCTGGAGATTTATGTTCT |
| | | ATGGAATCTTTTTATATTTAGGGGAAGTCACCAAAGCAGTACAGCCTCTCT |
| | | TACTGGGAAGAATCATAGCTTCCTATGACCCGGATAACAAGGAGGAACGC |
| | | TCTATCGCGATTTATCTAGGCATAGGCTTATGCCTTCTCTTTATTGTGAGGA |
| | | CACTGCTCCTACACCCAGCCATTTTTGGCCTTCATCACATTGGAATGCAGA |
| | | TGAGAATAGCTATGTTTAGTTTGATTTATAAGAAGACTTTAAAGCTGTCAAG |
| | | CCGTGTTCTAGATAAAATAAGTATTGGACAACTTGTTAGTCTCCTTTCCAAC |
| | | AACCTGAACAAATTTGATGAAGGACTTGCATTGGCACATTTCGTGTGGATC |
| | | GCTCCTTTGCAAGTGGCACTCCTCATGGGGCTAATCTGGGAGTTGTTACA |
| | | GGCGTCTGCCTTCTGTGGACTTGGTTTCCTGATAGTCCTTGCCCTTTTTCA |
| | | GGCTGGGCTAGGGAGAATGATGATGAAGTACAGAGATCAGAGAGCTGGG |
| | | AAGATCAGTGAAAGACTTGTGATTACCTCAGAAATGATCGAGAACATCCAA |
| | | TCTGTTAAGGCATACTGCTGGGAAGAAGCAATGGAAAAAATGATTGAAAAC |
| | | TTAAGACAAACAGAACTGAAACTGACTCGGAAGGCAGCCTATGTGAGATA |
| | | CTTCAATAGCTCAGCCTTCTTCTTCTCAGGGTTCTTTGTGGTGTTTTTATCT |
| | | GTGCTTCCCTATGCACTAATCAAAGGAATCATCCTCCGGAAAATATTCACC |
| | | ACCATCTCATTCTGCATTGTTCTGCGCATGGCGGTCACTCGGCAATTTCCC |
| | | TGGGCTGTACAAACATGGTATGACTCTCTTGGAGCAATAAACAAAATACAG |
| | | GATTTCTTACAAAAGCAAGAATATAAGACATTGGAATATAACTTAACGACTA |
| | | CAGAAGTAGTGATGGAGAATGTAACAGCCTTCTGGGAGGAGGGATTTGGG |
| | | GAATTATTTGAGAAAGCAAAACAAAACAATAACAATAGAAAAACTTCTAATG |
| | | GTGATGACAGCCTCTTCTTCAGTAATTTCTCACTTCTTGGTACTCCTGTCCT |
| | | GAAAGATATTAATTTCAAGATAGAAAGAGGACAGTTGTTGGCGGTTGCTGG |
| | | ATCCACTGGAGCAGGCAAGACTTCACTTCTAATGATGATTATGGGAGAACT |
| | | GGAGCCTTCAGAGGGTAAAATTAAGCACAGTGGAAGAATTTCATTCTGTTC |
| | | TCAGTTTTCCTGGATTATGCCTGGCACCATTAAAGAAAATATCATCTTTGGT |
| | | GTTTCCTATGATGAATATAGATACAGAAGCGTCATCAAAGCATGCCAACTA |
| | | GAAGAGGACATCTCCAAGTTTGCAGAGAAAGACAATATAGTTCTTGGAGAA |
| | | GGTGGAATCACACTGAGTGGAGGTCAACGAGCAAGAATTTCTTTAGCAAG |
| | | AGCAGTATACAAAGATGCTGATTTGTATTTATTAGACTCTCCTTTTGGATAC |
| | | CTAGATGTTTTAACAGAAAAAGAAATATTTGAAAGCTGTGTCTGTAAACTGA |
| | | TGGCTAACAAAACTAGGATTTTGGTCACTTCTAAAATGGAACATTTAAAGAA |
| | | AGCTGACAAAATATTAATTTTGCATGAAGGTAGCAGCTATTTTTATGGGACA |
| | | TTTTCAGAACTCCAAAATCTACAGCCAGACTTTAGCTCAAAACTCATGGGA |
| | | TGTGATTCTTTCGACCAATTTAGTGCAGAAAGAAGAAATTCAATCCTAACTG |
| | | AGACCTTACACCGTTTCTCATTAGAAGGAGATGCTCCTGTCTCCTGGACAG |
| | | AAACAAAAAAACAATCTTTTAAACAGACTGGAGAGTTTGGGGAAAAAAGGA |
| | | AGAATTCTATTCTCAATCCAATCAACTCTACGCTTCAGGCACGAAGGAGGC |
| | | AGTCTGTCCTGAACCTGATGACACACTCAGTTAACCAAGGTCAGAACATTC |
| | | ACCGAAAGACAACAGCATCCACACGAAAAGTGTCACTGGCCCCTCAGGCA |
| | | AACTTGACTGAACTGGATATATATTCAAGAAGGTTATCTCAAGAAACTGGC |
| | | TTGGAAATAAGTGAAGAAATTAACGAAGAAGACTTAAAGGAGTGCCTTTTT |
| | | GATGATATGGAGAGCATACCAGCAGTGACTACATGGAACACATACCTTCG |
| | | ATATATTACTGTCCACAAGAGCTTAATTTTTGTGCTAATTTGGTGCTTAGTA |
| | | ATTTTTCTGGCAGAGGTGGCTGCTTCTTTGGTTGTGCTGTGGCTCCTTGGA |
| | | AACACTCCTCTTCAAGACAAAGGGAATAGTACTCATAGTAGAAATAACAGC |
| | | TATGCAGTGATTATCACCAGCACCAGTTCGTATTATGTGTTTTACATTTACG |
| | | TGGGAGTAGCCGACACTTTGCTTGCTATGGGATTCTTCAGAGGTCTACCA |
| | | CTGGTGCATACTCTAATCACAGTGTCGAAAATTTTACACCACAAAATGTTAC |
| | | ATTCTGTTCTTCAAGCACCTATGTCAACCCTCAACACGTTGAAAGCAGGTG |
| | | GGATTCTTAATAGATTCTCCAAAGATATAGCAATTTTGGATGACCTTCTGCC |
| | | TCTTACCATATTTGACTTCATCCAGTTGTTATTAATTGTGATTGGAGCTATA |
| | | GCAGTTGTCGCAGTTTTACAACCCTACATCTTTGTTGCAACAGTGCCAGTG |
| | | ATAGTGGCTTTTATTATGTTGAGAGCATATTTCCTCCAAACCTCACAGCAAC |
| | | TCAAACAACTGGAATCTGAAGGCAGGAGTCCAATTTTCACTCATCTTGTTA |
| | | CAAGCTTAAAAGGACTATGGACACTTCGTGCCTTCGGACGGCAGCCTTAC |
| | | TTTGAAACTCTGTTCCACAAAGCTCTGAATTTACATACTGCCAACTGGTTCT |
| | | TGTACCTGTCAACACTGCGCTGGTTCCAAATGAGAATAGAAATGATTTTTG |
| | | TCATCTTCTTCATTGCTGTTACCTTCATTTCCATTTTAACAACAGGAGAAGG |
| | | AGAAGGAAGAGTTGGTATTATCCTGACTTTAGCCATGAATATCATGAGTAC |
| | | ATTGCAGTGGGCTGTAAACTCCAGCATAGATGTGGATAGCTTGATGCGAT |
| | | CTGTGAGCCGAGTCTTTAAGTTCATTGACATGCCAACAGAAGGTAAACCTA |
| | | CCAAGTCAACCAAACCATACAAGAATGGCCAACTCTCGAAAGTTATGATTA |
| | | TTGAGAATTCACACGTGAAGAAAGATGACATCTGGCCCTCAGGGGGCCAA |
| | | ATGACTGTCAAAGATCTCACAGCAAAATACACAGAAGGTGGAAATGCCATA |
| | | TTAGAGAACATTTCCTTCTCAATAAGTCCTGGCCAGAGGGTGGGCCTCTTG |
| | | GGAAGAACTGGATCAGGGAAGAGTACTTTGTTATCAGCTTTTTTGAGACTA |
| | | CTGAACACTGAAGGAGAAATCCAGATCGATGGTGTGTCTTGGGATTCAATA |
| | | ACTTTGCAACAGTGGAGGAAAGCCTTTGGAGTGATACCACAGAAAGTATTT |
| | | ATTTTTTCTGGAACATTTAGAAAAAACTTGGATCCCTATGAACAGTGGAGTG |
| | | ATCAAGAAATATGGAAAGTTGCAGATGAGGTTGGGCTCAGATCTGTGATA |
| | | GAACAGTTTCCTGGGAAGCTTGACTTTGTCCTTGTGGATGGGGGCTGTGT |
| | | CCTAAGCCATGGCCACAAGCAGTTGATGTGCTTGGCTAGATCTGTTCTCA |
| | | GTAAGGCGAAGATCTTGCTGCTTGATGAACCCAGTGCTCATTTGGATCCA |
| | | GTAACATACCAAATAATTAGAAGAACTCTAAAACAAGCATTTGCTGATTGCA |
| | | CAGTAATTCTCTGTGAACACAGGATAGAAGCAATGCTGGAATGCCAACAAT |
| | | TTTTGGTCATAGAAGAGAACAAAGTGCGGCAGTACGATTCCATCCAGAAAC |
| | | TGCTGAACGAGAGGAGCCTCTTCCGGCAAGCCATCAGCCCCTCCGACAG |
| | | GGTGAAGCTCTTTCCCCACCGGAACTCAAGCAAGTGCAAGTCTAAGCCCC |
| | | AGATTGCTGCTCTGAAAGAGGAGACAGAAGAAGAGGTGCAAGATACAAGG |
| | | CTTTAGAGAGCAGCATAAATGTTGACATGGGACATTTGCTCATGGAATTGG |
| | | CAGGCCTAATAAAGAGCTCAGATGCATCGATCAGAGTGTGTTGGTTTTTTG |
| | | TGTGTACTGAGGAACCCCTAGTGATGGAGTTGGCCACTCCCTCTCTGCGC |
| | | GCTCGCTCGCTCACTGAGGCCGGGCGACCAAAGGTCGCCCGACGCCCG |
| | | GGCTTTGCCCGGGGGGCCTCAGTGAGCGAGCGAGCGCGCAGAGAGGGA |
| | | GTGGCC |
| 18 | pAV- | CCACTCCCTCTCTGCGCGCTCGCTCGCTCACTGAGGCCGCCCGGGCAAA |
| | F5tg83- | GCCCGGGCGTCGGGCGACCTTTGGTCGCCCGGCCTCAGTGAGCGAGCG |
| | hCFTR-dR | AGCGCGCAGAGAGGGAGTGGCCAACTCCATCACTAGGGGTTCCTCAGAT |
| | (flop ITR) | CTGAATTCGTGGTGAGCGTCTGGGCATGTCTGGGCATGTCTGGGCATGTC |
| | vector | TGGGCATGTCGGGCATTCTGGGCGTCTGGGCATGTCTGGGCATGTCTGG |
| | | GCATCTCGAGAACGGTGACGTGCACGCGTGGGCGGAGCCATCACGCAGG |
| | | TTGCTATATAAGCAGAGCTCGTTTAGTGAACCGTCAGAGTCGAGCCCGAG |
| | | AGACCATGCAGAGGTCGCCTCTGGAAAAGGCCAGCGTTGTCTCCAAACTT |
| | | TTTTTCAGCTGGACCAGACCAATTTTGAGGAAAGGATACAGACAGCGCCT |
| | | GGAATTGTCAGACATATACCAAATCCCTTCTGTTGATTCTGCTGACAATCTA |
| | | TCTGAAAAATTGGAAAGAGAATGGGATAGAGAGCTGGCTTCAAAGAAAAAT |
| | | CCTAAACTCATTAATGCCCTTCGGCGATGTTTTTTCTGGAGATTTATGTTCT |
| | | ATGGAATCTTTTTATATTTAGGGGAAGTCACCAAAGCAGTACAGCCTCTCT |
| | | TACTGGGAAGAATCATAGCTTCCTATGACCCGGATAACAAGGAGGAACGC |
| | | TCTATCGCGATTTATCTAGGCATAGGCTTATGCCTTCTCTTTATTGTGAGGA |
| | | CACTGCTCCTACACCCAGCCATTTTTGGCCTTCATCACATTGGAATGCAGA |
| | | TGAGAATAGCTATGTTTAGTTTGATTTATAAGAAGACTTTAAAGCTGTCAAG |
| | | CCGTGTTCTAGATAAAATAAGTATTGGACAACTTGTTAGTCTCCTTTCCAAC |
| | | AACCTGAACAAATTTGATGAAGGACTTGCATTGGCACATTTCGTGTGGATC |
| | | GCTCCTTTGCAAGTGGCACTCCTCATGGGGCTAATCTGGGAGTTGTTACA |
| | | GGCGTCTGCCTTCTGTGGACTTGGTTTCCTGATAGTCCTTGCCCTTTTTCA |
| | | GGCTGGGCTAGGGAGAATGATGATGAAGTACAGAGATCAGAGAGCTGGG |
| | | AAGATCAGTGAAAGACTTGTGATTACCTCAGAAATGATCGAGAACATCCAA |
| | | TCTGTTAAGGCATACTGCTGGGAAGAAGCAATGGAAAAAATGATTGAAAAC |
| | | TTAAGACAAACAGAACTGAAACTGACTCGGAAGGCAGCCTATGTGAGATA |
| | | CTTCAATAGCTCAGCCTTCTTCTTCTCAGGGTTCTTTGTGGTGTTTTTATCT |
| | | GTGCTTCCCTATGCACTAATCAAAGGAATCATCCTCCGGAAAATATTCACC |
| | | ACCATCTCATTCTGCATTGTTCTGCGCATGGCGGTCACTCGGCAATTTCCC |
| | | TGGGCTGTACAAACATGGTATGACTCTCTTGGAGCAATAAACAAAATACAG |
| | | GATTTCTTACAAAAGCAAGAATATAAGACATTGGAATATAACTTAACGACTA |
| | | CAGAAGTAGTGATGGAGAATGTAACAGCCTTCTGGGAGGAGGGATTTGGG |
| | | GAATTATTTGAGAAAGCAAAACAAAACAATAACAATAGAAAAACTTCTAATG |
| | | GTGATGACAGCCTCTTCTTCAGTAATTTCTCACTTCTTGGTACTCCTGTCCT |
| | | GAAAGATATTAATTTCAAGATAGAAAGAGGACAGTTGTTGGCGGTTGCTGG |
| | | ATCCACTGGAGCAGGCAAGACTTCACTTCTAATGATGATTATGGGAGAACT |
| | | GGAGCCTTCAGAGGGTAAAATTAAGCACAGTGGAAGAATTTCATTCTGTTC |
| | | TCAGTTTTCCTGGATTATGCCTGGCACCATTAAAGAAAATATCATCTTTGGT |
| | | GTTTCCTATGATGAATATAGATACAGAAGCGTCATCAAAGCATGCCAACTA |
| | | GAAGAGGACATCTCCAAGTTTGCAGAGAAAGACAATATAGTTCTTGGAGAA |
| | | GGTGGAATCACACTGAGTGGAGGTCAACGAGCAAGAATTTCTTTAGCAAG |
| | | AGCAGTATACAAAGATGCTGATTTGTATTTATTAGACTCTCCTTTTGGATAC |
| | | CTAGATGTTTTAACAGAAAAAGAAATATTTGAAAGCTGTGTCTGTAAACTGA |
| | | TGGCTAACAAAACTAGGATTTTGGTCACTTCTAAAATGGAACATTTAAAGAA |
| | | AGCTGACAAAATATTAATTTTGCATGAAGGTAGCAGCTATTTTTATGGGACA |
| | | TTTTCAGAACTCCAAAATCTACAGCCAGACTTTAGCTCAAAACTCATGGGA |
| | | TGTGATTCTTTCGACCAATTTAGTGCAGAAAGAAGAAATTCAATCCTAACTG |
| | | AGACCTTACACCGTTTCTCATTAGAAGGAGATGCTCCTGTCTCCTGGACAG |
| | | AAACAAAAAAACAATCTTTTAAACAGACTGGAGAGTTTGGGGAAAAAAGGA |
| | | AGAATTCTATTCTCAATCCAATCAACTCTACGCTTCAGGCACGAAGGAGGC |
| | | AGTCTGTCCTGAACCTGATGACACACTCAGTTAACCAAGGTCAGAACATTC |
| | | ACCGAAAGACAACAGCATCCACACGAAAAGTGTCACTGGCCCCTCAGGCA |
| | | AACTTGACTGAACTGGATATATATTCAAGAAGGTTATCTCAAGAAACTGGC |
| | | TTGGAAATAAGTGAAGAAATTAACGAAGAAGACTTAAAGGAGTGCCTTTTT |
| | | GATGATATGGAGAGCATACCAGCAGTGACTACATGGAACACATACCTTCG |
| | | ATATATTACTGTCCACAAGAGCTTAATTTTTGTGCTAATTTGGTGCTTAGTA |
| | | ATTTTTCTGGCAGAGGTGGCTGCTTCTTTGGTTGTGCTGTGGCTCCTTGGA |
| | | AACACTCCTCTTCAAGACAAAGGGAATAGTACTCATAGTAGAAATAACAGC |
| | | TATGCAGTGATTATCACCAGCACCAGTTCGTATTATGTGTTTTACATTTACG |
| | | TGGGAGTAGCCGACACTTTGCTTGCTATGGGATTCTTCAGAGGTCTACCA |
| | | CTGGTGCATACTCTAATCACAGTGTCGAAAATTTTACACCACAAAATGTTAC |
| | | ATTCTGTTCTTCAAGCACCTATGTCAACCCTCAACACGTTGAAAGCAGGTG |
| | | GGATTCTTAATAGATTCTCCAAAGATATAGCAATTTTGGATGACCTTCTGCC |
| | | TCTTACCATATTTGACTTCATCCAGTTGTTATTAATTGTGATTGGAGCTATA |
| | | GCAGTTGTCGCAGTTTTACAACCCTACATCTTTGTTGCAACAGTGCCAGTG |
| | | ATAGTGGCTTTTATTATGTTGAGAGCATATTTCCTCCAAACCTCACAGCAAC |
| | | TCAAACAACTGGAATCTGAAGGCAGGAGTCCAATTTTCACTCATCTTGTTA |
| | | CAAGCTTAAAAGGACTATGGACACTTCGTGCCTTCGGACGGCAGCCTTAC |
| | | TTTGAAACTCTGTTCCACAAAGCTCTGAATTTACATACTGCCAACTGGTTCT |
| | | TGTACCTGTCAACACTGCGCTGGTTCCAAATGAGAATAGAAATGATTTTTG |
| | | TCATCTTCTTCATTGCTGTTACCTTCATTTCCATTTTAACAACAGGAGAAGG |
| | | AGAAGGAAGAGTTGGTATTATCCTGACTTTAGCCATGAATATCATGAGTAC |
| | | ATTGCAGTGGGCTGTAAACTCCAGCATAGATGTGGATAGCTTGATGCGAT |
| | | CTGTGAGCCGAGTCTTTAAGTTCATTGACATGCCAACAGAAGGTAAACCTA |
| | | CCAAGTCAACCAAACCATACAAGAATGGCCAACTCTCGAAAGTTATGATTA |
| | | TTGAGAATTCACACGTGAAGAAAGATGACATCTGGCCCTCAGGGGGCCAA |
| | | ATGACTGTCAAAGATCTCACAGCAAAATACACAGAAGGTGGAAATGCCATA |
| | | TTAGAGAACATTTCCTTCTCAATAAGTCCTGGCCAGAGGGTGGGCCTCTTG |
| | | GGAAGAACTGGATCAGGGAAGAGTACTTTGTTATCAGCTTTTTTGAGACTA |
| | | CTGAACACTGAAGGAGAAATCCAGATCGATGGTGTGTCTTGGGATTCAATA |
| | | ACTTTGCAACAGTGGAGGAAAGCCTTTGGAGTGATACCACAGAAAGTATTT |
| | | ATTTTTTCTGGAACATTTAGAAAAAACTTGGATCCCTATGAACAGTGGAGTG |
| | | ATCAAGAAATATGGAAAGTTGCAGATGAGGTTGGGCTCAGATCTGTGATA |
| | | GAACAGTTTCCTGGGAAGCTTGACTTTGTCCTTGTGGATGGGGGCTGTGT |
| | | CCTAAGCCATGGCCACAAGCAGTTGATGTGCTTGGCTAGATCTGTTCTCA |
| | | GTAAGGCGAAGATCTTGCTGCTTGATGAACCCAGTGCTCATTTGGATCCA |
| | | GTAACATACCAAATAATTAGAAGAACTCTAAAACAAGCATTTGCTGATTGCA |
| | | CAGTAATTCTCTGTGAACACAGGATAGAAGCAATGCTGGAATGCCAACAAT |
| | | TTTTGGTCATAGAAGAGAACAAAGTGCGGCAGTACGATTCCATCCAGAAAC |
| | | TGCTGAACGAGAGGAGCCTCTTCCGGCAAGCCATCAGCCCCTCCGACAG |
| | | GGTGAAGCTCTTTCCCCACCGGAACTCAAGCAAGTGCAAGTCTAAGCCCC |
| | | AGATTGCTGCTCTGAAAGAGGAGACAGAAGAAGAGGTGCAAGATACAAGG |
| | | CTTTAGAGAGCAGCATAAATGTTGACATGGGACATTTGCTCATGGAATTGG |
| | | CAGGCCTAATAAAGAGCTCAGATGCATCGATCAGAGTGTGTTGGTTTTTTG |
| | | TGTGTACTGAGGAACCCCTAGTGATGGAGTTGGCCACTCCCTCTCTGCGC |
| | | GCTCGCTCGCTCACTGAGGCCGGGCGACCAAAGGTCGCCCGACGCCCG |
| | | GGCTTTGCCCGGGGGGCCTCAGTGAGCGAGCGAGCGCGCAGAGAGGGA |
| | | GTGGCCCCCCCCCCCCCCCCCCCTGCAGCCTGGCGTAATAGCGAAGAGG |
| | | CCCGCACCGATCGCCCTTCCCAACAGTTGCGCAGCCTGAATGGCGAATG |
| | | GACGCGCCCTGTAGCGGCGCATTAAGCGCGGCGGGTGTGGTGGTTACGC |
| | | GCAGCGTGACCGCTACACTTGCCAGCGCCCTAGCGCCCGCTCCTTTCGCT |
| | | TTCTTCCCTTCCTTTCTCGCCACGTTCGCCGGCTTTCCCCGTCAAGCTCTA |
| | | AATCGGGGGCTCCCTTTAGGGTTCCGATTTAGTGCTTTACGGCACCTCGA |
| | | CCCCAAAAAACTTGATTAGGGTGATGGTTCACGTAGTGGGCCATCGCCCT |
| | | GATAGACGGTTTTTCGCCCTTTGACGTTGGAGTCCACGTTCTTTAATAGTG |
| | | GACTCTTGTTCCAAACTGGAACAACACTCAACCCTATCTCGGTCTATTCTTT |
| | | TGATTTATAAGGGATTTTGCCGATTTCGGCCTATTGGTTAAAAAATGAGCT |
| | | GATTTAACAAAAATTTAACGCGAATTTTAACAAAATATTAACGCTTACAATTT |
| | | CCTGATGCGGTATTTTCTCCTTACGCATCTGTGCGGTATTTCACACCGCAT |
| | | ATGGTGCACTCTCAGTACAATCTGCTCTGATGCCGCATAGTTAAGCCAGCC |
| | | CCGACACCCGCCAACACCCGCTGACGCGCCCTGACGGGCTTGTCTGCTC |
| | | CCGGCATCCGCTTACAGACAAGCTGTGACCGTCTCCGGGAGCTGCATGTG |
| | | TCAGAGGTTTTCACCGTCATCACCGAAACGCGCGAGACGAAAGGGCCTCG |
| | | TGATACGCCTATTTTTATAGGTTAATGTCATGATAATAATGGTTTCTTAGAC |
| | | GTCAGGTGGCACTTTTCGGGGAAATGTGCGCGGAACCCCTATTTGTTTATT |
| | | TTTCTAAATACATTCAAATATGTATCCGCTCATGAGACAATAACCCTGATAA |
| | | ATGCTTCAATAATATTGAAAAAGGAAGAGTATGAGTATTCAACATTTCCGTG |
| | | TCGCCCTTATTCCCTTTTTTGCGGCATTTTGCCTTCCTGTTTTTGCTCACCC |
| | | AGAAACGCTGGTGAAAGTAAAAGATGCTGAAGATCAGTTGGGTGCACGAG |
| | | TGGGTTACATCGAACTGGATCTCAACAGCGGTAAGATCCTTGAGAGTTTTC |
| | | GCCCCGAAGAACGTTTTCCAATGATGAGCACTTTTAAAGTTCTGCTATGTG |
| | | GCGCGGTATTATCCCGTATTGACGCCGGGCAAGAGCAACTCGGTCGCCG |
| | | CATACACTATTCTCAGAATGACTTGGTTGAGTACTCACCAGTCACAGAAAA |
| | | GCATCTTACGGATGGCATGACAGTAAGAGAATTATGCAGTGCTGCCATAA |
| | | CCATGAGTGATAACACTGCGGCCAACTTACTTCTGACAACGATCGGAGGA |
| | | CCGAAGGAGCTAACCGCTTTTTTGCACAACATGGGGGATCATGTAACTCG |
| | | CCTTGATCGTTGGGAACCGGAGCTGAATGAAGCCATACCAAACGACGAGC |
| | | GTGACACCACGATGCCTGTAGCAATGGCAACAACGTTGCGCAAACTATTA |
| | | ACTGGCGAACTACTTACTCTAGCTTCCCGGCAACAATTAATAGACTGGATG |
| | | GAGGCGGATAAAGTTGCAGGACCACTTCTGCGCTCGGCCCTTCCGGCTG |
| | | GCTGGTTTATTGCTGATAAATCTGGAGCCGGTGAGCGTGGGTCTCGCGGT |
| | | ATCATTGCAGCACTGGGGCCAGATGGTAAGCCCTCCCGTATCGTAGTTAT |
| | | CTACACGACGGGGAGTCAGGCAACTATGGATGAACGAAATAGACAGATCG |
| | | CTGAGATAGGTGCCTCACTGATTAAGCATTGGTAACTGTCAGACCAAGTTT |
| | | ACTCATATATACTTTAGATTGATTTAAAACTTCATTTTTAATTTAAAAGGATC |
| | | TAGGTGAAGATCCTTTTTGATAATCTCATGACCAAAATCCCTTAACGTGAGT |
| | | TTTCGTTCCACTGAGCGTCAGACCCCGTAGAAAAGATCAAAGGATCTTCTT |
| | | GAGATCCTTTTTTTCTGCGCGTAATCTGCTGCTTGCAAACAAAAAAACCAC |
| | | CGCTACCAGCGGTGGTTTGTTTGCCGGATCAAGAGCTACCAACTCTTTTTC |
| | | CGAAGGTAACTGGCTTCAGCAGAGCGCAGATACCAAATACTGTTCTTCTAG |
| | | TGTAGCCGTAGTTAGGCCACCACTTCAAGAACTCTGTAGCACCGCCTACAT |
| | | ACCTCGCTCTGCTAATCCTGTTACCAGTGGCTGCTGCCAGTGGCGATAAG |
| | | TCGTGTCTTACCGGGTTGGACTCAAGACGATAGTTACCGGATAAGGCGCA |
| | | GCGGTCGGGCTGAACGGGGGGTTCGTGCACACAGCCCAGCTTGGAGCGA |
| | | ACGACCTACACCGAACTGAGATACCTACAGCGTGAGCATTGAGAAAGCGC |
| | | CACGCTTCCCGAAGGGAGAAAGGCGGACAGGTATCCGGTAAGCGGCAGG |
| | | GTCGGAACAGGAGAGCGCACGAGGGAGCTTCCAGGGGGAAACGCCTGG |
| | | TATCTTTATAGTCCTGTCGGGTTTCGCCACCTCTGACTTGAGCGTCGATTT |
| | | TTGTGATGCTCGTCAGGGGGGCGGAGCCTATGGAAAAACGCCAGCAACG |
| | | CGGCCTTTTTACGGTTCCTGGCCTTTTGCTGGCCTTTTGCTCACATGTTCT |
| | | TTCCTGCGTTATCCCCTGATTCTGTGGATAACCGTATTACCGCCTTTGAGT |
| | | GAGCTGATACCGCTCGCCGCAGCCGAACGACCGAGCGCAGCGAGTCAGT |
| | | GAGCGAGGAAGCGGAAGAGCGCCCAATACGCAAACCGCCTCTCCCCGCG |
| | | CGTTGGCCGATTCATTAATGCAGGCTGCAGGGGGGGGGGGGGGGGGG |
| 19 | Wild-type | MQRSPLEKASVVSKLFFSWTRPILRKGYRQRLELSDIYQIPSVDSADNLSEKLE |
| | human | REWDRELASKKNPKLINALRRCFFWRFMFYGIFLYLGEVTKAVQPLLLGRIIAS |
| | CFTR | YDPDNKEERSIAIYLGIGLCLLFIVRTLLLHPAIFGLHHIGMQMRIAMFSLIYKKTL |
| | protein | KLSSRVLDKISIGQLVSLLSNNLNKFDEGLALAHFVWIAPLQVALLMGLIWELLQ |
| | | ASAFCGLGFLIVLALFQAGLGRMMMKYRDQRAGKISERLVITSEMIENIQSVKA |
| | | YCWEEAMEKMIENLRQTELKLTRKAAYVRYFNSSAFFFSGFFVVFLSVLPYALI |
| | | KGIILRKIFTTISFCIVLRMAVTRQFPWAVQTWYDSLGAINKIQDFLQKQEYKTL |
| | | EYNLTTTEVVMENVTAFWEEGFGELFEKAKQNNNNRKTSNGDDSLFFSNFSL |
| | | LGTPVLKDINFKIERGQLLAVAGSTGAGKTSLLMVIMGELEPSEGKIKHSGRISF |
| | | CSQFSWIMPGTIKENIIFGVSYDEYRYRSVIKACQLEEDISKFAEKDNIVLGEGG |
| | | ITLSGGQRARISLARAVYKDADLYLLDSPFGYLDVLTEKEIFESCVCKLMANKT |
| | | RILVTSKMEHLKKADKILILHEGSSYFYGTFSELQNLQPDFSSKLMGCDSFDQF |
| | | SAERRNSILTETLHRFSLEGDAPVSWTETKKQSFKQTGEFGEKRKNSILNPIN |
| | | SIRKFSIVQKTPLQMNGIEEDSDEPLERRLSLVPDSEQGEAILPRISVISTGPTL |
| | | QARRRQSVLNLMTHSVNQGQNIHRKTTASTRKVSLAPQANLTELDIYSRRLS |
| | | QETGLEISEEINEEDLKECFFDDMESIPAVTTWNTYLRYITVHKSLIFVLIWCLVI |
| | | FLAEVAASLVVLWLLGNTPLQDKGNSTHSRNNSYAVIITSTSSYYVFYIYVGVA |
| | | DTLLAMGFFRGLPLVHTLITVSKILHHKMLHSVLQAPMSTLNTLKAGGILNRFS |
| | | KDIAILDDLLPLTIFDFIQLLLIVIGAIAVVAVLQPYIFVATVPVIVAFIMLRAYFLQT |
| | | SQQLKQLESEGRSPIFTHLVTSLKGLWTLRAFGRQPYFETLFHKALNLHTANW |
| | | FLYLSTLRWFQMRIEMIFVIFFIAVTFISILTTGEGEGRVGIILTLAMNIMSTLQWA |
| | | VNSSIDVDSLMRSVSRVFKFIDMPTEGKPTKSTKPYKNGQLSKVMIIENSHVKK |
| | | DDIWPSGGQMTVKDLTAKYTEGGNAILENISFSISPGQRVGLLGRTGSGKSTL |
| | | LSAFLRLLNTEGEIQIDGVSWDSITLQQWRKAFGVIPQKVFIFSGTFRKNLDPY |
| | | EQWSDQEIWKVADEVGLRSVIEQFPGKLDFVLVDGGCVLSHGHKQLMCLAR |
| | | SVLSKAKILLLDEPSAHLDPVTYQIIRRTLKQAFADCTVILCEHRIEAMLECQQF |
| | | LVIEENKVRQYDSIQKLLNERSLFRQAISPSDRVKLFPHRNSSKCKSKPQIAAL |
| | | KEETEEEVQDTRL |

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method of treating cystic fibrosis (CF) in a subject whose genotype comprises at least one class I CFTR mutation, the method comprising administering to the subject a therapeutically effective amount of a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

2. A method of treating CF in a subject lacking CFTR protein, the method comprising administering to the subject a therapeutically effective amount of a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

3. The method of claim 2, wherein the subject's genotype comprises at least one class I CFTR mutation.

4. The method of claim 1 or 3, wherein the at least one class I CFTR mutation is a nonsense mutation, a splice mutation, or a deletion.

5. The method of any one of claims 1, 3, and 4, wherein the at least one class I CFTR mutation comprises a Q2X mutation, a S4X mutation, a W19X mutation, a G27X mutation, a Q39X mutation, a W57X mutation, a E60X mutation, a R75X mutation, a L88X mutation, a E92X mutation, a Q98X mutation, a Y122X mutation, a E193X mutation, a W216X mutation, a L218X mutation, a Q220X mutation, a Y275X mutation, a C276X mutation, a Q290X mutation, a G330X mutation, a W401X mutation, a Q414X mutation, a S434X mutation, a S466X mutation, a S489X mutation, a Q493X mutation, a W496X mutation, a C524X mutation, a Q525X mutation, a G542X mutation, a G550X mutation, a Q552X mutation, a R553X mutation, a E585X mutation, a G673X mutation, a Q685X mutation, a R709X mutation, a K710X mutation, a Q715X mutation, a L732X mutation, a R764X mutation, a R785X mutation, a R792X mutation, a E822X mutation, a W882X mutation, a W846X mutation, a Y849X mutation, a R851X mutation, a Q890X mutation, a S912X mutation, a Y913X mutation, a Q1042X mutation, a W1089X mutation, a Y1092X mutation, a W1098X mutation, a R1102X mutation, a E1104X mutation, a W1145X mutation, a R1158X mutation, a R1162X mutation, a S1196X mutation, a W1204X mutation, a L1254X mutation, a S1255X mutation, a W1282X mutation, a Q1313X mutation, a Q1330X mutation, a E1371X mutation, a Q1382X mutation, a Q1411X mutation, a 2116delCTAA mutation, or a combination thereof.

6. The method of any one of claims 1 and 3-5, wherein the subject's genotype comprises two class I CFTR mutations.

7. The method of claim 6, wherein the subject's genotype comprises a W1282X mutation and a R1162X mutation.

8. A method of treating CF in a subject whose genotype comprises at least one class III CFTR mutation, the method comprising administering to the subject a therapeutically effective amount of a recombinant adeno-associated virus (rAAV) comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

9. The method of claim 7, wherein the at least one class III CFTR mutation comprises a G551 D mutation or a S549N mutation.

10. The method of claim 8 or 9, wherein the subject's genotype comprises two class III CFTR mutations.

11. The method of any one of claims 1-10, further comprising administering to the subject a therapeutically effective amount of an augmenter of AAV transduction.

12. The method of claim 11, wherein the augmenter is administered to the subject within about 48 h following administration of the rAAV.

13. A method of treating CF in a subject, the method comprising:
(a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and
(b) administering to the subject a therapeutically effective amount of an augmenter of AAV transduction within about 48 h following administration of the rAAV.

14. A method of treating CF in a subject, the method comprising administering to the subject a therapeutically effective amount of an augmenter of AAV transduction, wherein the augmenter is administered to the subject within about 48 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

15. The method of any one of claims 12-14, wherein the augmenter is administered to the subject within about 24 h following administration of the rAAV.

16. The method of claim 15, wherein the augmenter is administered to the subject within about 12 h following administration of the rAAV.

17. The method of any one of claims 11-16, wherein the augmenter is an anthracycline, a proteasome inhibitor, a tripeptidyl aldehyde, or a combination thereof.

18. The method of claim 17, wherein the anthracycline is doxorubicin, idarubicin, aclarubicin, daunorubicin, epirubicin, valrubicin, mitoxantrone, or a combination thereof.

19. The method of claim 18, wherein the anthracycline is doxorubicin, idarubicin, or a combination thereof.

20. The method of claim 19, wherein the anthracycline is doxorubicin.

21. The method of claim 17, wherein the proteasome inhibitor is bortezomib, carfilzomib, and ixazomib.

22. The method of claim 17, wherein the tripeptidyl aldehyde is N-acetyl-1-leucyl-1-leucyl-1-norleucine (LLnL).

23. The method of any one of claims 13-22, wherein the subject lacks CFTR protein.

24. The method of any one of claims 13-23, wherein the subject's genotype comprises at least one class I CFTR mutation.

25. The method of claim 24, wherein the at least one class I CFTR mutation is a nonsense mutation, a splice mutation, or a deletion.

26. The method of claim 24 or 25, wherein the at least one class I CFTR mutation comprises a Q2X mutation, a S4X mutation, a W19X mutation, a G27X mutation, a Q39X mutation, a W57X mutation, a E60X mutation, a R75X mutation, a L88X mutation, a E92X mutation, a Q98X mutation, a Y122X mutation, a E193X mutation, a W216X mutation, a L218X mutation, a Q220X mutation, a Y275X mutation, a C276X mutation, a Q290X mutation, a G330X mutation, a W401X mutation, a Q414X mutation, a S434X mutation, a S466X mutation, a S489X mutation, a Q493X mutation, a W496X mutation, a C524X mutation, a Q525X mutation, a G542X mutation, a G550X mutation, a Q552X mutation, a R553X mutation, a E585X mutation, a G673X mutation, a Q685X mutation, a R709X mutation, a K710X mutation, a Q715X mutation, a L732X mutation, a R764X mutation, a R785X mutation, a R792X mutation, a E822X mutation, a W882X mutation, a W846X mutation, a Y849X mutation, a R851X mutation, a Q890X mutation, a S912X mutation, a Y913X mutation, a Q1042X mutation, a W1089X mutation, a Y1092X mutation, a W1098X mutation, a R1102X mutation, a E1104X mutation, a W1145X mutation, a R1158X mutation, a R1162X mutation, a S1196X mutation, a W1204X mutation, a L1254X mutation, a S1255X mutation, a W1282X mutation, a Q1313X mutation, a Q1330X mutation, a E1371X mutation, a Q1382X mutation, a Q1411X mutation, a 2116delCTAA mutation, or a combination thereof.

27. The method of any one of claims 24-26, wherein the subject's genotype comprises two class I CFTR mutations.

28. The method of claim 27, wherein the subject's genotype comprises a W1282X mutation and a R1162X mutation.

29. The method of any one of claims 13-26, wherein the subject's genotype comprises at least one class II CFTR mutation, at least one class III CFTR mutation, at least one class IV CFTR mutation, at least one class V CFTR mutation, at least one class VI CFTR mutation, or at least one class VII CFTR mutation.

30. The method of claim 29, wherein the subject's genotype comprises two class II CFTR mutations, two class III CFTR mutations, two class IV CFTR mutations, two class V CFTR mutations, two class VI CFTR mutations, or two class VII CFTR mutations.

31. The method of any one of claims 1-30, wherein the rAAV comprises an AV.TL65 capsid protein.

32. The method of claim 31, wherein the AV.TL65 capsid protein comprises the amino acid sequence of SEQ ID NO:13.

33. The method of any one of claims 1-32, wherein the polynucleotide comprises an F5 enhancer.

34. The method of claim 33, wherein the F5 enhancer comprises the polynucleotide sequence of SEQ ID NO:1.

35. The method of claim 33, wherein the F5 enhancer comprises the polynucleotide sequence of SEQ ID NO:14.

36. The method of any one of claims 1-35, wherein the polynucleotide comprises a tg83 promoter.

37. The method of claim 36, wherein the tg83 promoter comprises the polynucleotide sequence of SEQ ID NO:2.

38. The method of any one of claims 1-37, wherein the CFTRΔR minigene is a human CFTRΔR minigene.

39. The method of claim 38, wherein the human CFTRΔR minigene is encoded by a polynucleotide comprising the sequence of SEQ ID NO:4.

40. The method of any one of claims 1-39, wherein the polynucleotide comprises, in a 5′-to-3′ direction, the F5 enhancer, the tg83 promoter, and the CFTRΔR minigene.

41. The method of claim 40, wherein the polynucleotide comprises the sequence of SEQ ID NO:7.

42. The method of any one of claims 1-41, further comprising administering one or more additional therapeutic agents to the subject.

43. The method of claim 42, wherein the one or more additional therapeutic agents includes an antibiotic, a mucus thinner, a CFTR modulator, a mucolytic, normal saline, hypertonic saline, an immunosuppressive agent, or a combination thereof.

44. The method of any one of claims 1-43, wherein the administering is by inhalation, nebulization, aerosolization, intranasally, intratracheally, intrabronchially, orally, intravenously, subcutaneously, or intramuscularly.

45. The method of claim 44, wherein the administering is by inhalation, nebulization, aerosolization, intranasally, intratracheally, and/or intrabronchially.

46. The method of claim 45, wherein the administering is by inhalation.

47. An rAAV for use in treating CF in a subject whose genotype comprises at least one class I CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

48. An rAAV for use in treating CF in a subject lacking CFTR protein, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

49. An rAAV for use in treating CF in a subject whose genotype comprises at least one class III CFTR mutation, wherein the rAAV comprises (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.

50. An rAAV for use in a method of treating CF in a subject, the method comprising:
(a) administering to the subject a therapeutically effective amount of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof; and
(b) administering to the subject a therapeutically effective amount of an augmenter of AAV transduction within about 48 h following administration of the rAAV.

51. An augmenter of AAV transduction for use in treating CF in a subject, wherein the augmenter is administered to the subject within about 48 h following administration of an rAAV comprising (i) an AV.TL65 capsid protein or a variant thereof; and (ii) a polynucleotide comprising an F5 enhancer, or a variant thereof, and a tg83 promoter, or a variant thereof, operably linked to a CFTRΔR minigene or a variant thereof.
